(19) United States
(12) Patent Application Publication
(10) Pub. No.: US 2019/0167589 A1
(43) Pub. Date: Jun. 6, 2019

(54) BIOMIMETIC PROTEOLIPID VESICLE COMPOSITIONS AND USES THEREOF

(71) Applicant: The Methodist Hospital, Houston, TX (US)

(72) Inventors: Ennio Tasciotti, Houston, TX (US); Roberto Molinaro, Houston, TX (US)

(21) Appl. No.: 16/109,235

(22) Filed: Aug. 22, 2018

Related U.S. Application Data
(63) Continuation of application No. PCT/US2017/018991, filed on Feb. 22, 2017.
(60) Provisional application No. 62/298,339, filed on Feb. 22, 2016.

(51) Int. Cl. A61K 9/127; A61K 9/00; A61K 47/69; A61P 35/00

(52) U.S. Cl. CPC A61K 9/1272; A61K 9/0092; A61K 31/573; A61P 35/00; A61K 47/6917

(57) ABSTRACT

Disclosed are biomimetic proteolipid nanovesicles that possess remarkable properties for targeting compounds of interest to particular mammalian cell and tissue types. In particular embodiments, drug delivery vehicles are provided composed of synthetic phospholipids and cholesterol, enriched of leukocyte membranes, and surrounding an aqueous core. These nanovesicles are able to both avoid the immune system, thanks to the presence on their surface of self-tolerance proteins, as CD-45, CD-47, and MHC-1, and target inflamed endothelium, thereby diffusing in the tumor microenvironment. These properties make the composition highly suited for targeted drug delivery to mammalian tumor cells in vitro and in situ.

### CROSS-REFERENCE TO RELATED APPLICATIONS

The present application is a continuation of PCT Intl. Pat. Appl. No. PCT/US2017/018991; filed Feb. 22, 2017 (pending; Atty. Dkt. No. 37182.194WO01), which claims priority to U.S. Provisional Patent Application No. 62/298,339, filed Feb. 22, 2016 (expired; Atty. Dkt. No. 37182.194PV01); the contents of which is specifically incorporated herein in its entirety by express reference thereto.

### STATEMENT REGARDING FEDERALLY SPONSORED RESEARCH OR DEVELOPMENT

This invention was made with government support under Grant Nos. 1R-21-CA173579-01A1, 1R-03-DA035193, and 5U-54CA143837 awarded by the National Institutes of Health, and W81XWH-12-10414 awarded by the Department of Defense. The government has certain rights in the invention.

### NAMES OF THE PARTIES TO A JOINT RESEARCH AGREEMENT

Not Applicable.

### BACKGROUND OF THE INVENTION

### Field of the Invention

The present invention relates generally to the field of medicine, and in particular, to drug delivery compositions and formulations thereof. Disclosed are biomimetic proteolipid nanovesicles that possess remarkable properties for targeting compounds of interest to particular mammalian cell and tissue types. In particular embodiments, drug delivery vehicles are provided composed of synthetic phospholipids and cholesterol, enriched of leukocyte membranes, and surrounding an aqueous core. These nanovesicles are able to both avoid the immune system, thanks to the presence on their surface of self-tolerance proteins, as CD-45, CD-47, and MHC-1, and target inflamed endothelium, thereby diffusing in the tumor microenvironment. These properties make the composition highly suited for targeted drug delivery to mammalian tumor cells in vitro and in situ.

### Description of Related Art

A primary directive of nanotechnology is to develop drug delivery platforms that effectively reduce systemic toxicity of currently used drugs while retaining their pharmacological activity. To this purpose, several organic [lipids (Torchilin, 2014); polymers (Mitragotri et al., 2014)] and inorganic [silicon (Tasciotti et al., 2008), silica (Parodi et al., 2014); gold (Mura et al., 2013); and iron oxide (Kudgus et al., 2014) materials have been manipulated at the micro- and nano-scale to synthesize drug carriers. In the development of such materials bio-inspired approaches have emerged to face the extraordinary ability of the human body to recognize, label, sequester, and clear foreign objects (Luk and Zhang, 2015). By combining the typical properties of conventional synthetic nanoparticles (Ferrari, 2005; Torchilin, 2014) with natural (Hu et al., 2011; Hu et al., 2015; Parodi et al., 2013) or biomimetic (Hammer et al., 2008; Doshi et al., 2012) materials, such strategies revolutionized the concept of drug delivery in nanomedicine (Blanco et al., 2015).

In this scenario, bottom-up approaches opened the door to the synthesis of bio-inspired delivery systems through surface functionalization with targeting molecules that mimic plasma membrane receptors of specialized cells. Pioneering in this field was the design and development of Leuko-polymersomes that reconstituted two important leukocyte-derived adhesion molecules (analogs of P-selectin glycoprotein ligand 1 (PSGL-1) and leukocyte function-associated antigen (LFA) 1 receptors) on the surface of polymersomes (Robbins et al., 2010). The presence of these two receptor mimetics was shown to ensure avid and highly selective binding to the inflamed endothelium both in vitro and in vivo, thus suggesting that the contribution of both pathways is fundamental to mimic the leukocyte targeting properties (Robbins et al., 2010).

Using a similar approach, platelet-like nanoparticles displaying surface-binding, site-selective adhesion, and aggregation properties effectively mimicking platelets and their hemostatic functions, were synthesized (Anselmo et al., 2014). Although bottom-up approaches have the great advantage of providing superior physicochemical control over the final formulation, current chemical conjugation methods remain inadequate to reproduce the complexity of the cellular membrane on the surface of nanocarriers (Luk and Zhang, 2015). In fact, the contemporary addition of distinctive elements on the surface of drug carriers requires complex synthesis and purification protocols, whose complexity increases as a function of the number of different components.

To further bridge the gap between synthetic nanoparticles and biological materials, top-down procedures were developed. In this scenario, the use of plasma membranes derived from various cell types [i.e. red blood cells (Hu et al., 2011); platelets (Hu et al., 2015); leukocytes (Parodi et al., 2013); stem cells (Toledano-Furman et al., 2013)] to coat the surface of synthetic particles (Parodi et al., 2013) or used as carriers per se [cell ghosts for instance (Hu et al., 2011; 2015)], permitted the faithful recapitulation of the cellular biological complexity on the carrier's surface. The resulting biomimetic coating offers a one-step solution to simultaneously bestow particles with multiple bioactive functions including evasion of the mononuclear phagocytic system (MPS) and negotiation across various biological barriers (Yoo et al., 2011; Alvarez-Lorenzo and Concheiro, 2013).

The exploitation of leukocytes' features has been previously explored with the Leuko-like vector (LLV), and the use of circulating blood cells for drug delivery has already been shown, not only with leukocytes, but also with red blood cells, macrophages, platelets, and T cells. All of these approaches though, have limited, if any, translational potential due to the difficulty of the synthetic route, the limited access to the donor cells, the ability to consistently reproduce desired composition/features. In line with these other approaches, previous work has shown only that it was possible to transfer cell functions to a synthetic particle through the surface modification of silicon microparticles with patches of membranes. That process, however, had intrinsic limitations in the control of the coating procedure, yield, and scale-up.

### Deficiencies in the Prior Art

Unfortunately, top-down approaches also have their own limitations, such as issues in the control of physical parameters (i.e., size homogeneity) of the final formulation, poor control of the encapsulation and retention of chemically different molecules (i.e. hydrophilic, amphiphilic, and lipophilic small drugs), as well as difficulties involving the absence of a standardized protocol for preparation, contamination, and storage (Gutiérrez Milián et al., 2012; Millan et al., 2004).

### BRIEF SUMMARY OF THE INVENTION

The present invention overcomes these and other limitations inherent in the prior art by providing methods for the development of biomimetic nanovesicles, termed leukosomes, which employ proteins derived from leukocyte plasma membranes integrated into a synthetic biocompatible phospholipid bilayer as a new drug delivery platform. The leukosomes described herein provide improved drug delivery biomemtic vesicles that combine the cell properties of leukocytes with the drug-delivery features of liposomes.

In particular embodiments, the biomimetic nanovesicles of the present disclosure have been employed to provide combinational therapy of siRNAs and chemotherapeutic drugs to treat one or more forms of cancer. These drug delivery compositions improve the accumulation of conventional drugs in selected mammalian tissues, and achieve better therapeutic effect over currently-available therapies.

The present disclosure provides in one aspect, biomimetic nanovesicles that improve stability and loading doses for mammalian administration.

These nanovesicles can be used to preferentially target particular cell types, reduce phlogosis in a localized model of inflammation, while retaining both the versatility and physicochmiecal properties typical of conventional nanovesicle formulations.

### Chemotherapeutic Methods and Use

Another important aspect of the present disclosure concerns methods for using the disclosed drug delivery formulations for treating or ameliorating the symptoms of one or more forms of cancer, including, for example, a metastatic cancer, such as melanoma metastasis to the mammalian lung. Such methods generally involve administering to a mammal (and in particular, to a human in need thereof), one or more of the disclosed drug delivery systems comprising at least a first anticancer composition, in an amount and for a time sufficient to treat (or, alternatively ameliorate one or more symptoms of) the identified cancer in an affected mammal.

In certain embodiments, the nanovesicle formulations described herein may be provided to the animal in a single treatment modality (either as a single administration, or alternatively, in multiple administrations over a period of from several hours (hrs) to several days (or even several weeks or several months) as needed to treat the particular cancer. Alternatively, in some embodiments, it may be desirable to continue the treatment, or to include it in combination with one or more additional modes of therapy, for a period of several months or longer. In other embodiments, it may be desirable to provide the therapy in combination with one or more existing, or conventional treatment regimens.

The present disclosure also provides for the use of one or more of the disclosed nanovesicle drug delivery compositions in the manufacture of a medicament for therapy and/or for the amelioration of one or more symptoms of cancer, and particularly for use in the manufacture of a medicament for treating and/or ameliorating one or more symptoms of a mammalian cancer, including human cancers.

The present invention also provides for the use of one or more of the disclosed drug delivery nanovesicle formulations in the manufacture of a medicament for the treatment of cancer, and in particular, the treatment of human cancers.

Therapeutic Kits

Therapeutic kits including one or more of the disclosed nanovesicle drug delivery compositions and instructions for using the kit in a particular cancer treatment modality also represent preferred aspects of the present disclosure. These kits may further optionally include one or more additional anti-cancer compounds, one or more diagnostic reagents, one or more additional therapeutic compounds, or any combination thereof.

The kits of the invention may be packaged for commercial distribution, and may further optionally include one or more delivery devices adapted to deliver the micro/nano composite drug delivery composition(s) to an animal (e.g., syringes, injectables, and the like). Such kits typically include at least one vial, test tube, flask, bottle, syringe, or other container, into which the micro/nano composite drug delivery composition(s) may be placed, and preferably suitably aliquotted. Where a second pharmaceutical is also provided, the kit may also contain a second distinct container into which this second composition may be placed. Alternatively, the plurality of leukosomes disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Pharmaceutical Formulations

In certain embodiments, the present invention concerns formulation of one or more chemotherapeutic and/or diagnostic compounds in a pharmaceutically acceptable formulation of the leukosome compositions disclosed herein for administration to one or more cells or tissues of an animal, either alone, or in combination with one or more other modalities of diagnosis, prophylaxis, and/or therapy. The formulation of pharmaceutically acceptable excipients and carrier solutions is well known to those of ordinary skill in the art, as is the development of suitable dosing and treatment regimens for using the particular compositions described herein in a variety of treatment regimens.

In certain circumstances it will be desirable to deliver the disclosed chemotherapeutic compositions in suitably-formulated pharmaceutical vehicles by one or more standard delivery devices, including, without limitation, subcutaneously, parenterally, intravenously, intramuscularly, intrathecally, orally, intraperitoneally, transdermally, topically, by oral or nasal inhalation, or by direct injection to one or more cells, tissues, or organs within or about the body of an animal.

The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, transdermal, subdermal, and/or intraperitoneal administration. In this regard, the leukosome compositions of the present invention may be formulated in one or more pharmaceutically acceptable vehicles, including for example sterile aqueous media, buffers, diluents, etc. For example, a given dosage of active ingredient(s) may be dissolved in a particular volume of an isotonic solution (e.g., an isotonic NaCl-based solution), and then injected at the proposed site of administration, or further diluted in a vehicle suitable for intravenous infusion (see, e.g., “REMINGTON'S PHARMACEUTICAL SCIENCES” 15^{th} Ed., pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

Sterile injectable leukosome compositions may be prepared by incorporating the disclosed chemotherapeutic delivery system formulations in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The compositions disclosed herein may also be formulated in a neutral or salt form.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of chemotherapeutics disclosed herein will be within the purview of the ordinary-skilled artisan having benefit of the present teaching. It is likely, however, that the administration of a therapeutically-effective (i.e., a pharmaceutically-effective, chemotherapeutically-effective, or an anticancer-effective) amount of the disclosed leukosome drug delivery formulations may be achieved by a single administration, such as, without limitation, a single injection of a sufficient quantity of the delivered agent to provide the desired benefit to the patient undergoing such a procedure. Alternatively, in other circumstances, it may be desirable to provide multiple, or successive administrations of leukosome compositions disclosed herein, over relatively short or even relatively prolonged periods, as may be determined by the medical practitioner overseeing the administration of such compositions to the selected individual.

Typically, the leukosome compositions described herein will contain at least a chemotherapeutically-effective amount of a first active agent. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80 weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological t₁₁₂, route of administration, product shelf life, as well as other pharmacological considerations will be contemplated by one of ordinary skill in the art of preparing such pharmaceutical formulations, and as such, a variety of dosages and treatment regimens may be desirable.

Administration of the leukosome compositions disclosed herein may be administered by any effective method, including, without limitation, by parenteral, intravenous, intramuscular, or even intraperitoneal administration as described, for example, in U.S. Pat. Nos. 5,543,158; 5,641,515; and 5,399,363 (each of which is specifically incorporated herein in its entirety by express reference thereto). Solutions of the active compounds as free-base or pharmacologically acceptable salts may be prepared in water suitably mixed with a surfactant, such as hydroxypropylcellulose, or other similar fashion. The pharmaceutical forms adapted for injectable administration include sterile aqueous solutions or dispersions, and sterile powders for the extemporaneous preparation of sterile injectable solutions or dispersions including without limitation those described in U.S. Pat. No. 5,466,468 (specifically incorporated herein in its entirety by express reference thereto). In all cases, the form must be sterile and must be fluid to the extent that easy syringability exists. It must be at least sufficiently stable under the conditions of manufacture and storage, and must be preserved against the contaminating action of microorganisms, such as viruses, bacteria, fungi, and such like.

Exemplary carrier(s) may include, for example, a solvent or dispersion medium, including, without limitation, water, ethanol, polyol (e.g., glycerol, propylene glycol, and liquid polyethylene glycol, and the like, or a combination thereof), one or more vegetable oils, or any combination thereof, although additional pharmaceutically-acceptable components may be included.

Proper fluidity of the pharmaceutical formulations disclosed herein may be maintained, for example, by the use of a coating, such as e.g., a lecithin, by the maintenance of the required particle size in the case of dispersion, by the use of a surfactant, or any combination of these techniques. The inhibition or prevention of the action of microorganisms can be brought about by one or more antibacterial or antifungal agents, for example, without limitation, a paraben, chlorobutanol, phenol, sorbic acid, thimerosal, or the like. In many cases, it will be preferable to include an isotonic agent, for example, without limitation, one or more sugars or sodium chloride, or any combination thereof. Prolonged absorption of the injectable compositions can be brought about by the use in the compositions of agents delaying absorption, for example without limitation, aluminum monostearate, gelatin, or a combination thereof.

While systemic administration is contemplated to be effective in many embodiments of the invention, it is also contemplated that formulations disclosed herein be suitable for direct injection into one or more organs, tissues, or cell types in the body. Direct organ administration of the disclosed leukosome compositions may be conducted using suitable means, including those known to those of ordinary skill in the oncological arts.

The pharmaceutical formulations of the leukosome compositions disclosed herein are not in any way limited to use only in humans, or even to primates, or mammals. In certain embodiments, the methods and drug delivery formulations disclosed herein may be employed using avian, amphibian, reptilian, or other animal species. In preferred embodiments, however, the drug delivery compositions of the present invention are preferably formulated for administration to a mammal, and in particular, to humans, as part of an oncology regimen for treating one or more cancers. The leukosome compositions disclosed herein may also be acceptable for veterinary administration, including, without limitation, to selected livestock, exotic or domesticated animals, companion animals (including pets and such like), non-human primates, as well as zoological or otherwise captive specimens, and the like.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the disclosure. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, and FIG. 1F show exemplary leukosome synthesis and formulation in accordance with one aspect of the present disclosure. FIG. 1A: Extraction of proteolipid material from murine J774 macrophages. FIG. 1B: Protein enrichment of the phospholipid film. FIG. 1C: Vesicular formulation of Leukosomes. FIG. 1D: DSC analysis* of leukosomes and liposomes revealed a change in bilayer transition temperature (Tₘ) after membrane proteins incorporation. FIG. 1E: Deformability index evaluation* demonstrated leukosome bilayer's packing as a function of the protein-to-lipid ratio from 1:600 to 1:300. No change in vesicle deformability was noted at a protein-to-lipid ratio of 1:100. FIG. 1F: Schematic of membrane proteins' incorporation and of vesicle deformability dynamics. D₁: vesicle diameter before extrusion; D_{2,3,4}: vesicle diameter after extrusion (D1>D2>D3>D4). *All values are the average of at least 7 different measurements±SD. **p<0.01;

FIG. 2A, FIG. 2B, FIG. 2C, FIG. 2D, FIG. 2E, FIG. 2F, FIG. 2G, FIG. 2H, FIG. 2I, and FIG. 2J show characterization of leukosomes physicochemical features in accordance with one aspect of the present disclosure. DLS and cryoTEM analysis of FIG. 2A leukosomes and FIG. 2B, liposomes showed size, zeta potential, polydispersity index values, and size homogeneity of the two formulations (Scale bar 100 nm). High magnification cryoTEM micrographs of leukosomes (FIG. 2C) and liposomes (FIG. 2D) reveal a spherical shape for both vesicles, and a thicker bilayer for leukosomes (Scale bar 50 nm). FIG. 2E: Quantification of lipid bilayer showed a 1.6-fold increase of membrane thickness for leukosomes respect to liposomes. Atomic force microscopy images of FIG. 2F: liposomes and FIG. 2G, leukosomes reveals the presence of hinged structures on leukosome surface. FIG. 2H, Quantification of single particles' surface roughness (Ra) showed a 4-fold increase in leukosomes. FIG. 2I, ATR/FTIR spectrum of J774 membrane (black), liposomes (green), and leukosomes (red), confirmed the presence of protein components of the J774 membrane in the leukosomes' bilayer—broad band around ≈1620 cm⁻¹ (highlighted by the dotted line). FIG. 2J, Wheat Germ Agglutinin assay showed the presence of glycosylated proteins on the leukosome surface. Liposomes and membranes were used as negative and positive control, respectively. *p<0.05; **p<0.01;***p<0.001;

FIG. 3A, FIG. 3B, FIG. 3C, FIG. 3D, FIG. 3E, and FIG. 3F show analysis of the leukocyte membrane proteins transferred to the leukosome's lipid bilayer in accordance with one aspect of the present disclosure. FIG. 3A: Number of total and plasma membrane-associated proteins identified in the leukosome formulation. FIG. 3B is a schematic representation of the types of membrane and membrane-associated proteins identified. Integral proteins penetrate the membrane, while the peripheral ones are attached to one side. Cytoskeletal proteins are connected to plasma membranes thanks to the action of structural proteins that serve as anchors. Lipid anchored proteins are covalently bonded through a fatty acid to the plasma membrane. Secreted proteins are cycled between the outside and the inside of the cell through vesicles-mediated secretory pathways. FIG. 3C and FIG. 3D, Pie charts of the proteins identified in the Leukosome classified according to UniProt/GO information and manually searching in literature; FIG. 3C, classification of sub-classes of plasma membrane-associated proteins and FIG. 3D, functional characterization of the integral and lipid-anchored plasma membranes; FIG. 3E, schematic representation of leukosome bilayer enriched with molecules involved in transport, signaling, immunity, and adhesion; FIG. 3F, Flow cytometry analysis validates LFA-1, Mac-1, CD18, PSGL-1, CD45, and CD47 presence and their correct orientation on the surface of leukosomes' surface. The incubation of fluorescently labeled IgG with both liposomes and leukosomes revealed the absence of any unspecific binding of the antibody with vesicles' surface, thus indicating the high selectivity of the assay. **P<0.01; ***p<0.001;

FIG. 4A-1, FIG. 4A-2, FIG. 4A-3, FIG. 4B-1, FIG. 4B-2, FIG. 4C-1, FIG. 4C-2, FIG. 4D-1, and FIG. 4D-2 show leukosomes retain drug loading and release properties similar to control liposomes in accordance with one aspect of the present disclosure. FIG. 4A-1, FIG. 4A-2, FIG. 4A-3: Dexamethasone, caffeine and paclitaxel molecular formula and their water solubility are reported. They are representative of hydrophilic, amphiphilic, and hydrophobic drugs, respectively. FIG. 4B-1, FIG. 4B-2, FIG. 4C-1, FIG. 4C-2, and FIG. 4D-1 and FIG. 4D-2: Encapsulation efficiency and in vitro release profile of dexamethasone (FIG. 4B-1 and FIG. 4B-2), caffeine (FIG. 4C-1 and FIG. 4C-2), and paclitaxel (FIG. 4D-1 and FIG. 4D-2)-loaded liposomes (GREEN) and leukosomes (RED). Leukosomes showed loading properties similar to conventional liposomes, while they delayed the release of their payload;

FIG. 5A-1, FIG. 5A-2 FIG. 5A-3, FIG. 5A-4, FIG. 5A-5, FIG. 5A-6, FIG. 5A-7, FIG. 5A-8, FIG. 5B-1, FIG. 5B-2, FIG. 5B-3, FIG. 5B-4, FIG. 5B-5, FIG. 5B-6, FIG. 5B-7, FIG. 5C, FIG. 5D-1, FIG. 5D-2, FIG. 5D-3, FIG. 5D-4, FIG. 5D-5, FIG. 5D-6, FIG. 5D-7, and FIG. 5E show leukosomes preferentially adhere to inflamed vasculature in vivo and improve tissue healing by preserving its architecture and reducing neutrophil infiltration in accordance with one aspect of the present disclosure. FIG. 5A-1, FIG. 5A-2 FIG. 5A-3, FIG. 5A-4, FIG. 5A-5, FIG. 5A-6, FIG. 5A-7, FIG. 5A-8: IVM images of inflamed-vasculature targeting relative to rhodamine-labeled liposomes (green) and leukosomes (red). Compared to control liposomes, leukosomes showed a 5-fold and 8-fold increased accumulation into ear tissue at 1- and 24-hr after particles' injection, respectively. FIG. 5B-1, FIG. 5B-2, FIG. 5B-3, FIG. 5B-4, FIG. 5B-5, FIG. 5B-6, FIG. 5B-7: Histological analysis of not inflamed (control) and inflamed (free DXM, empty and DXM-loaded liposomes, and empty and DXM-loaded leukosomes) ear tissues shows an alteration of tissue architecture in the untreated group and in the ones treated with free DXM, empty and DXM-loaded liposomes. FIG. 5C: Inspection of ear cryo-sections revealed a significantly reduced thickness for the groups treated with leukosomes (empty and DXM-loaded) (p<0.001) compared to the other groups. No statistically significant difference was observed among the control and the leukosomes-treated groups. FIG. 5D-1, FIG. 5D-2, FIG. 5D-3, FIG. 5D-4, FIG. 5D-5, FIG. 5D-6, FIG. 5D-7: Immunofluorescence analysis of ear sections at 24 hrs reveals that both empty and DXM-loaded leukosomes exhibited a significant reduction in neutrophil infiltration in ear tissue compared to the other groups. FIG. 5E: Fluorescence intensity quantification of labeled neutrophils is reported. *p<0.1; **p<0.01;***p<0.001. Scale bar=100 μm;

FIG. 6A-1, FIG. 6A-2, FIG. 6A-3, FIG. 6B-1, FIG. 6B-2, FIG. 6B-3, FIG. 6B-4, FIG. 6B-5, FIG. 6B-6, FIG. 6B-7, FIG. 6B-8, FIG. 6B-9, FIG. 6B-10, FIG. 6B-11, FIG. 6B-12, FIG. 6B-13, FIG. 6B-14, FIG. 6B-15, FIG. 6B-16, FIG. 6B-17, FIG. 6B-18, FIG. 6C-1, FIG. 6C-2, FIG. 6C-3, FIG. 6C-4, FIG. 6D, and FIG. 6E show the immunogenicity and safety of leukosomes in accordance with one aspect of the present disclosure. FIG. 6A-1, FIG. 6A-2, FIG. 6A-3: Serum levels of the main cytokines (IL-6, TNFα, and IL-1β) in mice (n=5) treated with a high dosage of leukosomes (1000 mg/Kg). Blood samples were collected 1 and 7 days after leukosome i.v. administration. FIG. 6B-1, FIG. 6B-2, FIG. 6B-3, FIG. 6B-4, FIG. 6B-5, FIG. 6B-6, FIG. 6B-7, FIG. 6B-8, FIG. 6B-9, FIG. 6B-10, FIG. 6B-11, FIG. 6B-12, FIG. 6B-13, FIG. 6B-14, FIG. 6B-15, FIG. 6B-16, FIG. 6B-17, FIG. 6B-18: Representative haematoxylin and eosin stained sections of indicated organs from mice 1 week after systemic injection of leukosomes, Liposomes, and PBS (CONTROL). FIG. 6C-1, FIG. 6C-2, FIG. 6C-3: Flow cytometry profiles of IgG and IgM-positive liposomes and leukosomes, previously incubated with serum (primary antibody) of untreated (control) and treated mice. FACS analysis showed no observable elevation of autologous antibody titer compared with the control. In fact, very limited leukosomes, less than 3 and 0.3% were labeled by host serum and secondary antibodies (anti-IgM and IgG, respectively) (FIG. 6D and FIG. 6E), and the same trend can be observed with control liposomes. These results suggest that leukosomes do not initiate any strong adaptive immune response and antibody production against membrane antigens related to the particles;

FIG. 7A, FIG. 7B, and FIG. 7C show EM micrographs of leukocyte-derived membranes and storage stability of extracted membrane proteins. EM micrographs of leukocyte-derived membranes and storage stability of extracted membrane proteins. FIG. 7A: TEM and FIG. 7B: SEM images of purified leukocyte membranes; FIG. 7C: storage stability of membrane proteins evaluated as content of total proteins over 4 weeks storage at different temperatures;

FIG. 8A and FIG. 8B show bilayer profiles of high-magnification cryo-TEM images of representative liposomal and leukosomal vesicles. High-magnification cryo-TEM images of a liposomal (FIG. 8A) and a leukosomal (FIG. 8B) vesicle are shown along with corresponding line profiles through lipid bilayers. The vesicles were selected from pictures of both types of cryo-TEM samples taken with similar defocus values to ensure comparable imaging conditions. The analysis reveals a slight, but significant thicker bilayer for leukosomes with respect to liposomes;

FIG. 9A-1, FIG. 9A-2, FIG. 9B-1, FIG. 9B-2, and FIG. 9C illustrate heights representation and property map of Young's modulus of a representative sample of liposome and leukosome AFM images. Heights representation and property map of Young's modulus of a representative sample of liposome (FIG. 9A) and leukosome (FIG. 9B) images using AFM analysis. The elastic modulus for leukosomes (476±3.2 kPa) resulted slightly but significantly increased with respect to the one for liposomes (423±4.4 kPa). The increase in the Young's modulus corresponds to a higher stiffness of the leukosomes' bilayer compared to the liposomal one;

FIG. 10A and FIG. 10B show molecular weights (MW) and Score and Sequence Coverage (SC) distribution of the proteins identified in the leukosomes. FIG. 10A: Molecular weights (MW) distribution of the proteins identified in the leukosomes. The number of proteins falling into each MW range is plotted on the left y-axis, while the percentage of proteins is showed on the right y-axis. FIG. 10B: Score and Sequence Coverage (SC) distribution. Proteins classification according to their Score (blue scale) and Sequence Coverage (grey scale) obtained by mass spectrometry analysis. Distribution analysis reveals that most of the proteins have been identified with a score in the range 300-2000 and a sequence coverage between 10 and 30%;

FIG. 11A-1, FIG. 11A-2, FIG. 11A-3, FIG. 11A-4, and FIG. 11B show the validation of markers' expression on J774 surface. FIG. 11A-1, FIG. 11A-2, FIG. 11A-3, FIG. 11A-4: Immunofluorescence analysis of J774 macrophages stained with FITC-labeled anti-LFA-1, anti-MAC-1, and anti-CD45; FIG. 11B: Flow cytometry analysis validates LFA-1, CD45 and Mac-1 presence and their correct orientation on the surface of J774 macrophage. ***p<0.001;

FIG. 12A and FIG. 12B show the storage stability of liposomes and leukosomes at 4° C. was evaluated by DLS analysis. Average size (FIG. 12A) and polydispersity index (PDI) (FIG. 12B) were measured up to 4 weeks from assembly;

FIG. 13A and FIG. 13B show the physical characterization of drug-loaded leukosomes. DLS analysis revealed that caffeine, paclitaxel, and dexamethasone did not significantly affect leukosome physical properties (size and formulation homogeneity) (Table 3). A significant change in zeta potential values can be noted after paclitaxel encapsulation. PDI: polydispersity index;

FIG. 14 shows marker's expression on leukosome surface after drug loading. Dexamethasone (DXM) encapsulation did not affect the surface properties of leukosome. LFA-1, Mac-1, and CD45 presence and correct orientation on carrier's surface was evaluated through flow cytometry analysis of dexamethasone (DXM)-loaded leukosomes as above described. Each result is the average of 5 different measurements±SD;

FIG. 15A-1, FIG. 15A-2, FIG. 15A-3, FIG. 15A-4, and FIG. 15B show in vitro adhesion of liposomes and leukosomes in flow condition to a reconstructed endothelium made by HUVEC cells. No statistically significant difference in adhesion was found between liposomes and leukosomes in not inflamed conditions, while after pre-treatment of HUVEC cells with TNFα, leukosome targeting was significantly higher than liposomes. ****p<0.0001;

FIG. 16A and FIG. 16B show the PCR analysis of pro (CCR-2, and IL-6), anti (MRC-1)-inflammatory markers, and endothelial adhesion molecules (ICAM-1 and VCAM-1) expression. Heat map study shows how leukosomes were significantly more efficient than DXM free (p<0.001) and loaded into liposomes (p<0.01) in reducing the expression of pro-inflammatory genes and of the adhesion molecules ICAM1 and VCAM1, typically over-expressed in case of vascular inflammation and responsible of the subsequent leukocytes' binding. In addition, MRC-1 gene levels resulted increased after leukosome treatment compared to free and liposome-loaded DXM (p<0.001);

FIG. 17 depicts bioluminescence imaging of mice to confirm local inflammation. 24 hrs after administration of LPS on the left ears of mice. Mice were treated with 5 mg/mouse of luminol. BLI analysis shows luminol signals originating only from the right ear while the left ear (control) had negligible signal. This imaging confirmed that inflammation was restricted to the right ears with prominent recruitment of neutrophils;

FIG. 18A-1, FIG. 18A-2, FIG. 18B-1, FIG. 18B-2, FIG. 18B-3, FIG. 18C-1, FIG. 18C-2, and FIG. 18C-3 illustrate particles' distribution into the ear at 1 and 24 hr after systemic injection. FIG. 18A-1, FIG. 18A-2: Liposomes and leukosomes accumulation into the inflamed ear tissue at 1- and 24-hr after injection. FIG. 18B-1, FIG. 18B-2, FIG. 18B-3: Liposomes are more abundant into the extravascular space at 1 h, as a result of the EPR effect occurring at the vascular level following the LPS-induced inflammation, while leukosomes are associated to the vasculature, due to their active-targeting properties. FIG. 18C-1, FIG. 18C-2, and FIG. 18C-3: At 24-hrs, liposomes were in equilibrium between the two environments, while leukosomes gradually crossed the vascular barrier accumulating into the extravascular space. (Scale bar=50 μm);

FIG. 19A-1, FIG. 19A-2, FIG. 19A-3, FIG. 19B, FIG. 19C-1, FIG. 19C-2, FIG. 19C-3, and FIG. 19C-4 illustrate the in vitro mechanisms of adhesion of leukosomes after either LFA-1 or CD45 blocking in flow condition to a reconstructed endothelium made by HUVEC cells pretreated with TNFα. Compare to control leukosomes, a significant reduction of particles' adhesion can be observed after blocking of either LFA-1 (α-LFA-1) and CD45 (α-CD45) on leukosome surface, thus confirming that the adhesion is mainly regulated by LFA-1, and validating the cooperative effect between these two markers. ***p<0.005; ****p<0.001. Stars represent relative to liposome. Dots represent relative to Leukosome;

FIG. 20A-1, FIG. 20A-2, FIG. 20A-3, FIG. 20A-4, and FIG. 20B illustrate the in vivo mechanisms of particles' adhesion to the inflamed endothelium. The figure shows the targeting abilities of leukosomes toward LPS-inflamed ear after blocking of either LFA-1 or CD45 on their surface. The blocking of either LFA-1 or CD45 nullifies the targeting abilities of leukosomes, which, as a result, show a significantly reduced adhesion with respect to the control leukosomes. In addition, no statistically significant difference can be observed among anti-LFA-1, or anti-CD45-leukosomes and liposomes, thus indicating that, after blocking, the only mechanism of accumulation remains the passive targeting. ***p<0.001; ****p<0.0001;

FIG. 21 shows the biodistribution and pharmacokinetics study of liposomes and leukosomes after 24 hr from i.v. injection. Mice (n=5 for each group) received 2 mg of rhodamine-labeled liposomes and leukosomes. 30 μL blood was collected from the retro orbital plexus at the time points and the rhodamine-related fluorescence was quantified for fluorescence;

FIG. 22 shows macroscopic observation of inflamed ears. Representative left (control) and right (treated) ears of mice (n=3) were harvested and punched. Macroscopic observation revealed the classical signs of inflammation, calor, rubor, and tumor (heat, redness, and swelling), clearly visible by eye, and further macroscopically investigated;

FIG. 23A and FIG. 23B demonstrate liver and kidney functionality. Blood test parameters after administration of leukosomes, liposomes and PBS (mean±SD, n=3) revealed how leukosomes did not induce any change in liver (ALP, ALT, and AST) and kidney (BUN) functionality with respect to PBS and liposome groups;

FIG. 24 shows an exemplary method for organic solvent-mediated reconstitution of liposome particles. Liposome assembly procedures include ethanol injection, ether infusion, and reverse-phase evaporation. Large proteoliposomes (5-300 μm). Incorporation of single proteins (rhodopsin, cytochrome c oxidase, acetylcholine receptor). Drawbacks include: organic solvents denature amphiphilic membrane proteins;

FIG. 25 shows an exemplary method for reverse-phase evaporation for preparation of liposomes. Large proteoliposomes (0.2-5 μm); incorporation of single proteins (rhodopsin, bacteriorhodopsin); Drawbacks: the lack of general procedures for the transfer into apolar solvents of other more hydrophilic membrane proteins in an active form has precluded the genera 1 use of this method, which should, in any case, be assessed with very hydrophobic proteins;

FIG. 26 shows various exemplary mechanical means for incorporation of proteins into liposomes, including single proteins such as rhodopsin, and bacteriorhodopsin, etc.;

FIG. 27 shows an exemplary method for thin-layer evaporation method of leukosome assembly in accordance with one aspect of the present disclosure. Liposome assembly procedures include, for example, reverse-phase evaporation; permits the incorporation of single proteins (e.g., Ca²⁺-ATPase); Drawbacks: incomplete protein incorporation and broad size distribution;

FIG. 28 shows an exemplary method for thin-layer evaporation method of leukosome assembly in accordance with one aspect of the present disclosure. Liposome assembly procedures include, for example, Thin-layer evaporation methods; Proteoliposomes (120 nm)-polydispersity index <0.1 (high homogeneity); incorporation of multiple proteins (342 according to preteomic analysis);

FIG. 29A, FIG. 29B, FIG. 29C, FIG. 29C, FIG. 29D, and FIG. 29E show particle characterization. (FIG. 29A) SEM images of uncoated particles (NPS) and particles coated with cellular membrane derived from murine macrophages (J774 LLV) and human T-cells (Jurkat LLV). (FIG. 29B) Fluorescent microscope images of LLV-modified with Alexa Fluor 555 (red, first column) and immunofluorescent staining of Jurkat LLV and J774 LLV for surface markers LFA-1 and Mac-1 (green, second column) and merged (third column). (FIG. 29C) Western blot analysis of leukocyte adhesion molecules successfully transferred on LLV (WCL: whole cell lysate). (FIG. 29D) Flow cytometry analysis of particles revealing the presence of LFA-1 and Mac-1. (FIG. 29E) Flow cytometry analysis of the particles stained for wheat germ agglutinin. The data are plotted as the mean±s.d;

FIG. 30A and FIG. 30B show CAM-1 pathway activation schematic. Activation of ICAM-1 pathway by LLV: (FIG. 30A) LLV adhere to inflamed endothelium interacting with ICAM-1 through adhesive receptors LFA-1 and Mac-1 (see inset). This interaction is efficient in activating the ICAM-1 pathway. Subsequently, ICAM-1 pathway activation results in an increase in intracellular calcium and ROS concentrations, resulting in an independent activation of PKCα. PKCα increases lead to the phosphorylation of VE-cadherin, resulting in the disassembly of VE-cadherin and protein displacement. (FIG. 30B) Following protein displacement of VE-cadherin, gaps between endothelial cells form, leading to an increase in vascular permeability and payload transport into the extracellular matrix;

FIG. 31A, FIG. 31B, and FIG. 31C show adhesion proprieties and effect on calcium signaling in inflamed endothelium. (FIG. 31A) Representative images of NPS and LLV (red) adhered on endothelial cells following a brief flow of particles to discriminate between particles bound on the cell border and cell interior. VE-Cadherin junctions of endothelial cells were labeled with an anti-VE-cadherin antibody (green) and nuclei were stained with DAPI (blue) (scale bar: 25 μm). (FIG. 31B) Graph representing differential LLV and NPS distribution on cell border or interior. (FIG. 31C) Calcium signaling following particle flow was assessed through a Fluo3 AM staining monitored in live microscopy. The data are plotted as the mean±s.d;

FIG. 32A, FIG. 32B, FIG. 32C, and FIG. 32D show ICAM1 pathway activation. (FIG. 32A) Western blot analysis of Ve-cadherin-P and VE-cadherin 15 min. following particles and leukocytes treatment. (FIG. 32B) Quantitative analysis of VE-cadherin expression on cell border of TNFα-activated HUVEC treated with a flow of leukocytes or particles. Data were obtained by immunofluorescence. Fluorescence intensity was measured along the perimeter of HUVEC per condition (n=15). (FIG. 32C) Immunofluorescence images and tri-dimensional fluorescence intensity profile (3D Intensity) showing single TNFα-activated HUVEC. (FIG. 32D) Intensity profiles of the cell perimeter of single TNFα-activated HUVEC plotted in polar coordinates. For B, C and D images the analyses were performed on untreated HUVEC (CTRL) and on HUVEC treated with Jurkat cells (Leukocytes), uncoated particles (NPS), and coated particles (LLV). The data are plotted as the mean±s.d. Statistical analysis was performed using a one-way ANOVA with a Turkey post-test. Asterisks denote significance relative to CTRL. Dots denote significance relative to NPS. ****P<0.0001.

FIG. 33A, FIG. 33B, FIG. 33C, FIG. 33D, FIG. 33E, and FIG. 33F show intravital microscopy analysis of LLV tumor endothelium targeting and binding stability. (FIG. 33A) Intravital microscopy images of orthotopic 4T1 tumor following treatment with NPS and LLV (scale bar: 100 μm). (FIG. 33B) Quantification of particles bound to tumor vasculature, count was performed on same area fraction. (FIG. 33C) Intravital microscope images portraying binding stability of LLV and NPS on tumor endothelium at 1 and 2 hrs created by merging together consecutive frames obtained from 20 sec movies (scale bar: 50 μm). Arrows indicate new (red), stable (yellow), or detached (white) particles. (FIG. 33D) Quantification of binding stability determined from intravital microscope images. (FIG. 33E) Particle motion analysis in tumor vasculature. Plotting X or Y particles position as a function of time, firmly bound particle events appear as straight lines while moving particle events appear as askew lines. (F) In the Graph we report the registered velocity of moving NPS particles compared to LLV particles which appears all in steady state. The data are plotted as the mean±s.d; and

FIG. 34A and FIG. 34B show intravital microscopy analysis of 70 kDa dextran extravasation. (FIG. 34A) Tumor vasculature images of mice administered with dextran following NPS and LLV injection, (scale bar: 100 μm). Images were acquired over 45 min. Insets represent a heat map of yellow box to highlight dextran extravasation. (FIG. 34B) Quantitative analysis on relative fold change of dextran penetration into the subendothelial space. The data are plotted as the mean±s.d. Statistical analysis was performed using a two-way ANOVA with a Bonferroni post-test. **P<0.01.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

In the present application, methods are described for manipulation of the cell membrane, and its use as a biomaterial per se. Here, the cell membrane is not just a coating, but represents the core material making the whole delivery platform.

The main differences between the current methods, and with platforms developed to date can be summarized as follows:

Synthetic route. Leukocyte membranes are not used to coat a particle (top-down approach), but to self-assemble with synthetic phospholipids to create nano-sized composite vesicles (bottom-up). Also, the thin layer evaporation method has been used so far just to load hydrophilic drugs into liposomal core, never for the incorporation of cell-derived membrane proteins into a synthetic bilayer. In addition, protein incorporation into a liposomal bilayer served so far as powerful tool for elucidating both functional and structural aspects of these membrane-associated proteins, due to their role in the control of fundamental biochemical processes and their importance as pharmaceutical targets. Here, the inventors describe exploiting their ability to specifically bind to receptors and mediate carriers' functions.

Strategies for membrane protein reconstitution into liposomes include:

Organic solvent-mediated reconstitution: evaporation of a solution of protein-lipid complex in apolar solvents followed by rehydration with aqueous buffer (Darszon et al., 1980). Reverse-phase evaporation: proteoliposomes are formed from water-in-oil emulsion of phospholipid-protein-aqueous buffer in excess of organic solvent, followed by removal of the organic phase under reduced pressure (Szoka and Papahadjopoulos, 1978). Mechanical means: sonication, French press, freeze-thaw. Direct incorporation into preformed liposomes (Tomita et al., 1992; Jain and Zakim, 1987). Detergent-mediated reconstitutions: proteins are co-solubilized with phospholipids, then next detergent is removed resulting in the progressive formation of bilayer vesicles with incorporated proteins.

In contrast, leukosomes are prepared herein by the thin layer evaporation (TLE) method. The nature of the phospholipids used: mixtures of egg phosphatidylcholine and egg phosphatidic acid, or 1,2-dioleoyl-sn-glycerophosphocholine (DOPC) and 1,2-dioleoyl-sn-glycerophosphoethanolamine (DOPE), alone or combined with detergents, are commonly used. A combination of 1,2-dipalmitoyl-sn-glycerophosphocholine (DPPC), 1,2-distearoyl-sn-glycerophosphocholine (DSPC), Cholesterol, and DOPC is used for the incorporation of membrane proteins.

The lipid-to-protein ratio: the reconstitution strategies so far developed used a large range of ratios from about 160 to 5 (wt./wt.) for most proteins analyzed. In particular, only single proteins, or at the most coupled proteins, were reconstituted into synthetic bilayers. The current approach uses a lipid-to-protein ratio of 300, and more than 300 different proteins have been transferred into liposomal bilayer using the compositions described herein.

Pharmaceutical Formulations

In certain embodiments, the present invention concerns nanovesicle compositions prepared in pharmaceutically-acceptable formulations for delivery to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable surgical implantation methods for using the particular membrane compositions described herein in a variety of treatment regimens, and particularly those involving bone regrowth.

Sterile injectable formulations may be prepared by incorporating the disclosed leukosome-based drug delivery compositions in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The leukosome-based drug delivery compositions disclosed herein may also be formulated in solutions comprising a neutral or salt form to maintain the integrity of the vesicles prior to administration.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, implantation regimen, formulation, and prepartation of the leukosome-based drug delivery compositions disclosed herein will be within the purview of the ordinary-skilled artisan having benefit of the present teaching. It is likely, however, that the administration of a particular leukosome composition may be achieved by a single administration to provide the desired benefit to the patient undergoing such a procedure. Alternatively, in some circumstances, it may be desirable to provide multiple, or successive administrations of the leukosome-based agents, either over a relatively short, or even a relatively prolonged period, as may be determined by the medical practitioner overseeing the individual undergoing treatment.

The leukosome-based drug delivery compositions disclosed herein are not in any way limited to use only in humans, or even to primates, or mammals. In certain embodiments, the methods and leukosome-based drug delivery compositions disclosed herein may be employed in the treatment of avian, amphibian, reptilian, and/or other animal species, and may be formulated for veterinary surgical use, including, without limitation, for administration to selected livestock, exotic or domesticated animals, companion animals (including pets and such like), non-human primates, as well as zoological or otherwise captive specimens, and such like.

Compositions for the Preparation of Medicaments

Another important aspect of the present invention concerns methods for using the disclosed leukosome compositions (as well as formulations including them) in the preparation of medicaments for treating and/or ameliorating one or more symptoms of one or more diseases, dysfunctions, abnormal conditions, or disorders in an animal, including, for example, vertebrate mammals.

Such use generally involves administration to the mammal in need thereof one or more of the disclosed leukosome compositions, in an amount and for a time sufficient to treat or ameliorate one or more symptoms of an injury, defect, or disease in an affected mammal.

Pharmaceutical formulations including one or more of the disclosed leukosome compositions also form part of the present invention, and particularly those compositions that further include at least a first pharmaceutically-acceptable excipient for use in the therapy and/or amelioration of one or more symptoms of disease, defect, abnormal condition, or trauma in an affected mammal.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, “chondrocyte” shall mean a differentiated cell responsible for secretion of extracellular matrix of cartilage. Preferably, the cells are from a compatible human donor. More preferably, the cells are from the patient (i.e., autologous cells).

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

As used herein, “fibroblast” shall mean a cell of connective tissue that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. The fibroblast cell may be mesodermally derived, and secrete proteins and molecular collagen including fibrillar procollagen, fibronectin and collagenase, from which an extracellular fibrillar matrix of connective tissue may be formed. A “fibroblast-like cell” means a cell that shares certain characteristics with a fibroblast (such as expression of certain proteins).

The terms “for example” or “e.g.,” as used herein, are used merely by way of example, without limitation intended, and should not be construed as referring only those items explicitly enumerated in the specification.

As used herein, “hard tissue” is intended to include mineralized tissues, such as bone, teeth, and cartilage. Mineralized tissues are biological tissues that incorporate minerals into soft matrices.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

As used herein, “matrix” shall mean a three-dimensional structure fabricated with biomaterials. The biomaterials can be biologically-derived or synthetic.

As used herein, a “medical prosthetic device,” “medical implant,” “implant,” and such like, relate to a device intended to be implanted into the body of a vertebrate animal, such as a mammal, and in particular a human. Implants in the present context may be used to replace anatomy and/or restore any function of the body. Examples of such devices include, but are not limited to, dental implants and orthopedic implants. In the present context, orthopedic implants includes within its scope any device intended to be implanted into the body of a vertebrate animal, in particular a mammal such as a human, for preservation and restoration of the function of the musculoskeletal system, particularly joints and bones, including the alleviation of pain in these structures.

In the present context, dental implants include any device intended to be implanted into the oral cavity of a vertebrate animal, in particular a mammal such as a human, in tooth restoration procedures. Generally, a dental implant is composed of one or several implant parts. For instance, a dental implant usually comprises a dental fixture coupled to secondary implant parts, such as an abutment and/or a dental restoration such as a crown, bridge, or denture. However, any device, such as a dental fixture, intended for implantation may alone be referred to as an implant even if other parts are to be connected thereto. Orthopedic and dental implants may also be denoted as orthopedic and dental prosthetic devices as is clear from the above. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, “mesh” means a network of material. The mesh may be woven synthetic fibers, non-woven synthetic fibers, nanofibers, or any combination thereof, or any material suitable for implantation into a mammal, and in particular, for implantation into a human.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, “osteoblast” shall mean a bone-forming cell which forms an osseous matrix in which it becomes enclosed as an osteocyte. It may be derived from mesenchymal osteoprogenitor cells. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells. Preferably the cells are from a compatible human donor. More preferably, the cells are from the patient (i.e., autologous cells).

As used herein, “osteointegrative” means having the ability to chemically bond to bone.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “pharmaceutically-acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, “porosity” means the ratio of the volume of interstices of a material to a volume of a mass of the material.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, “scaffold,” relates to an open porous structure. A scaffold may comprise one or more building materials to create the structure of the scaffold. Additionally, the scaffold may further comprise other substances, such as one or more biologically active molecules or such like.

As used herein, “soft tissue” is intended to include tissues that connect, support, or surround other structures and organs of the body, not being bone. Soft tissue includes ligaments, tendons, fascia, skin, fibrous tissues, fat, synovial membranes, epithelium, muscles, nerves and blood vessels.

As used herein, “stem cell” means an unspecialized cell that has the potential to develop into many different cell types in the body, such as mesenchymal osteoprogenitor cells, osteoblasts, osteocytes, osteoclasts, chondrocytes, and chondrocyte progenitor cells. Preferably, the cells are from a compatible human donor. More preferably, the cells are from the patient (i.e., autologous cells).

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The tern “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

Biological Functional Equivalents

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| Amino Acids | | Codons | | | | | |
|---|---|---|---|---|---|---|---|
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Preparation of Leukosomes for Targeting Inflamed Tissues

In the last decades several micro and nano drug delivery systems were developed to control the transport of pharmaceuticals, biologics and theranostic agents within the human body. A multitude of micro- and nanoparticles have been developed to improve the delivery of systemically administered pharmaceuticals, which are subject to a number of biological barriers that limit their optimal biodistribution. Bio-inspired approaches have emerged as an alternative treatment capable of evading the mononuclear phagocytic system and facilitating transport across the endothelial vessel wall. In the last few decades, bottom-up and top-down approaches have been developed to formulate these carriers, revolutionizing the field of nanomedicine and inspiring the development of novel methods to face their potential drawbacks. In this example, a method is described that merges the advantages of both approaches through the formulation of proteins derived from leukocyte plasma membranes into nanovesicles. These leukosomes preferentially target inflamed vasculature both in vitro and in vivo, permitting the selective and effective delivery of dexamethasone, reducing phlogosis in a localized model of inflammation, while retaining the versatility and physicochemical properties typical of liposomal formulations. The present example demonstrates preparation of the biomimetic proteolipid vesicles, which, in accordance with one aspect of the present disclosure, may be used to improve delivery of one or more active agents to inflamed tissues within or about the body of an animal in need thereof.

In this study, a biomimetic vesicle, the leukosome, is described, which uses proteins derived from leukocyte plasma membranes integrated into a synthetic biocompatible phospholipid bilayer through an approach that merges the advantages of bottom-up and top-down strategies.

The leukosome concept is based on the most established nanotechnology to date, the liposomes. Since their first appearance in the literature, liposomes have been studied and used in multiple clinical applications for more than 30 years. Today, several liposomal formulations have been approved by FDA and are regularly used in the medical practice. Liposomes can carry chemotherapeutics (Doxil), antibiotics (AmBisome), pain killers (Embrel) and have been confirmed as the preferred carrier for the encapsulation of siRNA, a new class of therapeutics. The present invention deals with the assembly method of Leukosomes, biomimetic carriers derived from synthetic liposomes enriched with leukocyte membrane proteins, and their physical, molecular and biological characterization. Leukocyte membrane proteins confer reticulo endothelial system escape and tumor targeting properties to leukosomes, while maintaining the drug delivery properties typical of liposomes.

Leukosomes are biomimetic drug delivery vesicles composed of a bilayer, made by synthetic phospholipids and cholesterol, enriched of leukocyte membranes, surrounding an aqueous core. They are assembled in order to mimic the physiological capability of leukocytes, which are able to avoid the immune system, thanks to the presence on their surface of self-tolerance proteins, as CD-45, CD-47, and MHC-1, and to target the inflamed endothelium and to diffuse in the tumor microenvironment. This latter activity depends on the expression of adhesion proteins, as LFA-1, Mac-1, which recognize and bind to ICAM-1, over-expressed on the surface of inflamed endothelial cells, and promote leukocyte adhesion and the subsequent tissue infiltration. Leukosomes were formulated with purified cell membranes enriched with cholesterol and synthetic choline-based phospholipids: (1,2-dipalmitoyl-sn-glycero-3-phosphocholine (DPPC), 1,2-distearoyl-sn-glycero-3-phosphocholine (DSPC), cholesterol and dioleoyl-sn-glycero-3-phospho-(1′-rac-glycerol) (sodium salt) (DOPC) (5:1:1:3, molar ratio). Phospholipids were chosen in order to mimic the phosphatidilcholine enriched cell membrane composition, and to accommodate leukocyte membrane proteins at a protein:phospholipid ratio of 1:300 (wt./wt.).

Leukocytes freely circulate in the bloodstream and selectively target the inflamed vasculature in response to injury, infection, and cancer. Here the manipulation of a biological proteolipid material (i.e., proteins derived from the plasma membranes of leukocytes) is shown for the assembly of a biomimetic liposomal-based drug delivery system called leukosomes. This study effectively demonstrated both the design and manipulation of materials isolated from living cells to impart biological functions to synthetic nanoparticles.

Materials and Methods

Assembly and Physical Characterization of Leukosomes.

Leukosomes were prepared using the thin layer evaporation (TLE) method. Briefly, 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), and cholesterol (Avanti Polar Lipids) were dissolved in a chloroform:methanol mixture (3:1 vol./vol.) and the solvent was evaporated through a rotary evaporator (BÜCHI Labortechnik AG, SWITZERLAND) to form a film according to the well-established TLE procedure. Films were hydrated with a PBS dispersion of membrane proteins (1:300 protein-to-lipid ratio) or PBS to respectively assemble leukosomes or liposomes (control). Lipid suspension was extruded ten times through 200-nm pore-size cellulose acetate membranes at 45° C. Physical characterization was performed with a Nanosizer ZS (Malvern Instruments). For CryoTEM analysis, liposomes and leukosomes were plunge-frozen on holey film grids (R2×2 Quantifoil®; Micro Tools GmbH, Jena, GERMANY) as previously reported (Sherman et al., 2006). Images were acquired on a JEOL 2100 electron microscope under low electron-dose conditions (˜5-20 electrons/Å²) using a 4,096×4,096 pxl CCD camera (UltraScan 895, GATAN, Inc., nominal magnifications 20,000×). The proteomic profile was obtained via peptide-level LC/MS^{E} analysis by in-solution trypsin digestion after reduction and alkylation of disulfide bridges and de-lipidation with methanol/chloroform extraction. Bradford (Bio-Rad) protein assay was employed to determine protein concentration, followed by trypsin digestion (overnight at 37° C. with an enzyme:substrate=1:50 molar ratio). AFM images of the liposomes and leukosomes were collected in Scan Asyst® mode by Multimode (Bruker, Calif., USA) using single-beam silicon cantilever probes (Bruker MLCT): resonance frequency 10 KHz, nominal tip radius of curvature 10 nm, force constant of 0.04 N/m). Fourier Transform Infrared (FTIR) spectroscopy measurements in attenuated total reflection were performed using a single reflection diamond element. For this study, the FTIR spectrometer Nicolet was equipped with a nitrogen cooled mercury cadmium telluride detector. Leukosomes with different protein/lipid ratio (1:100, 1:300, 1:600) were prepared for DSC measurements using a Star DSC (Mettler-Toledo, Berne, SWITZERLAND), to evaluate the bilayer thermal transitions at increasing protein contents. Passive loading of leukosomes was obtained by hydrating the lipid film with a caffeine (1:10 caffeine to lipid ratio), or a dexamethasone (1:5 dexamethasone-to-lipid ratio) solution, or by dissolving paclitaxel (1:30 paclitaxel to lipid ratio) in the chloroform:methanol mixture containing the lipids.

In Vivo Confocal Imaging.

All animal experiments were performed in accordance with all federal, state, and institutional guidelines using approved protocols. IVM imaging was performed under anesthesia with isoflurane. Rhodamine-labeled particles (liposomes and leukosomes) were injected intravenously via retro-orbital injection. 70-kDa fluorescein isothiocyanate (FITC)-labeled dextran dye [5 mg/ml; 50 μL in PBS (Invitrogen, Carlsbad, Calif., USA)] was used as vessel tracer as previously reported (Parodi et al., 2013). IVM studies of leukosomes' dynamics were performed to determine effectiveness of tissue targeting and accumulation. Upon systemic administration, dynamic flow and real-time accumulation of liposomes and leukosomes were monitored for up to 60 min post-injection. Adhesion to the inflamed vasculature was monitored using an upright AIR laser scanning confocal microscope, equipped with resonance scanner, motorized and heated stage, and a Nikon long working distance 4× and 20× dry plan-apochromatic objectives. Images were obtained with a three-channel setup in which fluorescence was collected at 488/525 nm for FITC dextran, and at 561/579 nm for rhodamine-labeled particles. Image acquisition was performed over n=10 field of views (FOVs) at a resolution of 512×256 pixels with an optical slice thickness of 5 μm. To determine the extent of leukosomes and liposomes accumulation in the ear parenchyma, the animals were imaged 1 hr and 24 hr after i.v. injection (50 μL, 1 mg/mL). Images were analyzed using Nikon Elements software.

Physical Characterization of Leukosomes. Dynamic Light Scattering Analysis.

Vesicle's size and polydispersity index were determined through dynamic light scattering analysis using a Nanosizer ZS (Malvern Instruments) that permitted also to evaluate their surface charge. 20 μL of liposome and leukosome suspensions were diluted in bi-distilled water and seven measurements were performed with 20 runs each and the results averaged.

CryoEM Analysis.

For electron microscopy analysis, lipid vesicles were plunge-frozen on holey film grids (R2×2 Quantifoil®; Micro Tools GmbH, Jena, Germany) as previously reported². A 626 cryo-specimen holder (Gatan, Inc., Pleasanton, Calif.) was used for imaging. Data were collected on a JEOL 2100 electron microscope. Images were recorded under low electron-dose conditions (˜5-20 electrons/A2) using a 4,096×4,096 pixel CCD camera (UltraScan 895, GATAN, Inc.) at nominal magnifications of 20,000×.

Atomic Force Microscopy (AFM) Analysis.

AFM images of the liposomes and leukosomes were collected in Scan Asyst®mode by Multimode (Bruker, Calif., USA) using single-beam silicon cantilever probes (Bruker MLCT: resonance frequency 10 KHz, nominal tip radius of curvature 10 nm, force constant of 0.04 N/m). Data sets were subjected to a first-order flattening. The particles roughness (Ra), an arithmetic value that describes the absolute height of a surface in comparison to a two-dimensional plane represented by the average sample height was calculated using Nanoscope 6.13R1 software (Digital Instruments, NY, USA). Mean values from 30 random particles in 3 independent experiments are reported. In addition, quantitative analysis of the AFM force mapping was performed to evaluate the relative particles' elasticity. This technique directly measures the elastic properties of different surfaces resulting in a complete elastic property map of heterogeneous samples. Samples were prepared employing a 0.1% APTES coating of mica surface in order to stabilize the nanoparticles (avoiding their collapse on mica surface); AFM analysis was subsequently performed. The Young's modulus measurement was calculated for 3 different samples corresponding to 512×512 force-separation curves obtained over an area 10 μm×10 μm.

The Young's elastic modulus was calculated using the following equation previously reported

F−F_{adh}=4/3E′\sqrt{R(d−d0)}³

Histology of Ear Tissue.

Explanted mouse ears were washed twice with PBS and embedded in a cryomold in O.C.T. (Tissue-Tek® O.C.T. Compound, Sakura® Finetek), and instantly frozen at −80° C. Ten μm-thick slides were obtained cutting ears block with a cryostat at −20° C. The slides were stored at −20° C. For hematoxylin and eosin (H/E) staining, slides were thawed, hydrated, washed and stained with hematoxylin and eosin (Sigma-Aldrich).

Immunofluorescence Analysis of Ear Tissue.

Once cryo-sections were obtained as described in the previous paragraph, immunofluorescence (IF) staining was performed as previously reported (Gelain et al., 2010). Briefly, slides were thawed and blocked with BSA 5% (Sigma-Aldrich) PBS 1× solution. After washing, they were incubated overnight at 4° C. with anti-neutrophil antibody (Alexa Fluor® 647 anti-mouse Ly-6G/Ly-6C (Gr-1) Biolegend. Excess anti-neutrophil antibody was washed out with PBS 1X. Cells nuclei were stained with DAPI. Slides were sealed with ProLong Gold antifade reagent (Life Technologies™). Images were captured with an Eclipse® Ti Inverted Fluorescent Microscope equipped with Hamamatsu Digital Camera C11440 ORCA-Flash 2.8.

Immunogenicity and Safety of Leukosomes.

8-week-old BALB/C mice (n=5) were intravenously injected with leukosomes and control liposomes once per week for one month. Then, the blood was collected from the mice at 6 weeks after the last injection and the serum isolated as previously reported (Copp et al., 2014). The sera were used as primary antibody, and were incubated with the particles, which were first blocked with BSA and FBS at room temperature for 30 min. After one wash, anti-mouse IgM and IgG secondary antibodies labeled with different fluorochromes were incubated with particles at room temperature for 30 min. After washing, the particles were analyzed by FACS analysis IgM or IgG-positive particles indicated that specific antibodies were generated in the host blood against them.

Fourier Transform Infrared Spectroscopy (FTIR) Analysis.

FTIR measurements in attenuated total reflection (ATR) were performed using a single reflection diamond element. The FTIR spectrometer Nicolet equipped with nitrogen cooled mercury cadmium telluride detector and an air purging system, was employed under the following conditions: 2 cm⁻¹ spectral resolution, 20 kHz scan speed, 1000 scan co-addition, and triangular apodization. Each sample was dissolved at a final concentration of 1 mg/mL in PBS. 5 μL of each sample were deposited on the ATR plate and spectra were recorded after solvent evaporation to allow the formation of a hydrated lipid film. After these measurements, the same samples were re-suspended, and the spectra were recorded again after the solvent evaporation for three times in order to confirm the data. The ATR/FTIR spectra were reported after background subtraction and normalization on the C═O vibrational mode located at ≈, 1730 cm⁻¹ band area to compensate for possible differences in the lipid content.

Differential Scanning Calorimetry (DSC) Analysis.

Leukosomes with different protein/lipid ratio (1:100, 1:300, 1:600) were prepared for DSC measurements using a Mettler-Toledo Star DSC (Mettler-Toledo). Liposomes were used as control, to evaluate differences in the bilayer thermal transitions with increasing protein content. A concentrated aqueous suspension of the samples was placed in an alumina pan for analysis, and an empty pan was used as reference. The heating scan was from 25 to 60° C. at the rate of 5° C./min. DSC curves were analyzed using the fitting program.

Analysis of Leukosome Protein Composition. Sample Preparation and LC/MS^{E} Conditions.

Leukosomes were analyzed via peptide-level LC/MS^{E} analysis by in-solution trypsin digestion after reduction and alkylation of disulfide bridges as follows: Samples were resuspended with 0.5% RapiGest SF surfactant (Waters Corp, Milford, Mass., USA) in 50 mM ammonium bicarbonate (AMBIC) at pH 8.0, and then treated with 5 mM dithiothreitol (DTT) at 60° C. for 30 min, and with 15 mM iodoacetamide (IAA) at room temperature in the dark. Samples were de-lipidated with methanol/chloroform extraction. Bradford (Bio-Rad) protein assay was employed to determine protein concentration, then trypsin digestions were performed overnight at 37° C. (enzyme:substrate=1:50 molar ratio). Reactions were stopped by changing the pH of the solution, and by adding 0.5% of trifluoroacetic acid. A Waters Corp. NanoAcquity UPLC system coupled with a Synapt HDMS (G1) mass spectrometer was employed. The peptide mixtures were separated with a reverse phase C-18 column and then injected into the mass spectrometer in positive ion (ESI) mode. The LC system consisted of a 180 μmμ 20 mm Symmetry C₁₈ (5 μm particle) trapping column, and a 75 μm×250 mm BEH130 C-18 (1.7 μm particle) analytical column. Peptide separation was carried out by a 3-40% gradient of solvent B (0.1% formic acid in acetonitrile) in solvent A (0.1% formic acid in water) over 120 min at flow of 0.3 uL/min and a column temperature of 35° C. The mass spectrometer was operated in the data independent (parallel-ion fragmentation) MS^{E} mode (Silva et al., 2005; Silva et al., 2006; Geromanos et al., 2009) at a capillary voltage of 3 kV, with alternating low (6V) and ramped high collision energies (15V-45V) at a scan rate of 1.2 sec per scan. The Glu-fibrinopeptide B (GFP) was sampled every 30 secs as internal calibrant. All data were collected with the time of flight (TOF) detector set in the V-mode (resolution ˜10,000). All LC/MS instrument control and data acquisition was accomplished using MassLynx (v4.1) software from Waters Corp. The samples were analyzed in triplicate.

Protein Identification and Classification.

For protein identification and quantification ProteinLynx Global Server (PLGS v2.4; Waters Corp.) software was employed, using both the IdentityE and ExpressionE algorithms included in the software. Precursor ions and fragment ion mass error tolerance levels (typically less than 5 ppm and 15 ppm respectively) were calculated automatically by the software. The Uniprot 2013_03 (‘reviewed’) (16,614 entries) complete mouse proteome database was interrogated. The false discovery rate (FDR) for protein identifications was set at 1%. Peptide identifications were accepted with a minimum of 2 peptides and 7 fragment ions matched per protein, with a minimum of 4 fragment ions per peptide detected. As database search parameters, the following were selected: a) carbamidomethyl-cysteine as fixed modification, b) oxidized-methionine as variable modification, c) and one trypsin miscleavage. All protein identifications were further filtered to retain only those protein IDs that remained above the 95% confidence interval. The whole protein data set (Table) identified in the leukosomes was submitted to bioinformatics analysis and classified on the basis of biological process and cellular component. The proteins were classified according to UniProt/GO information and manually searching in literature.

Evaluation of Protein Orientation into Leukosome Bilayer.

Flow cytometry analysis was performed to validate the presence of leukocyte-derived membrane proteins and to confirm their correct orientation into leukosome bilayer. Leukosomes and liposomes were diluted in FACS Buffer (PBS, 1% BSA) to a final concentration of 0.5 mM and incubated separately with FITC-labeled anti-Mac-1 and anti-LFA-1, PerCP-labeled anti-CD45, PE-labeled PSGL-1 and CD18, and AlexaFluor 647-labeled CD47 (2.5 μg/mL) designed to bind the protein's extracellular domain for at least 30 min at room temperature. Samples were next dialyzed using 1000 kDa membrane filters for 1.5 hr in water covered from light with mild stirring and then analyzed on the flow cytometer. The analysis was performed also after dexamethasone loading to verify whether drug encapsulation affects surface properties.

Characterization of Protein Glycosylation.

Glycosylation of membrane-associated proteins was verified using the wheat germ agglutinin (WGA) assay (Life Technologies, San Diego, Calif.). WGA is a carbohydrate-binding protein that selectively binds N-acetyl-D-glucosamine and sialic acid glycosylated residues on the plasma membrane. Briefly, samples (liposomes, leukosomes and extracted and purified membrane proteins) were incubated at 1 μg/mL Alexa Fluor® 488-conjugated WGA in standard buffers (HBSS) for 10 min and then washed through dialysis. WGA fluorescence (excitation/emission maxima ˜495/519 nm) was measured spectrofluorometrically.

Evaluation of In Vitro Adhesion Ability of Leukosomes to a Reconstructed Endothelium.

Flow experiments were performed by seeding HUVEC cells onto ibidi μ-slide I^{0.4} LueribiTreat, tissue culture treated slides. Briefly, slides were incubated for 1 hr to equilibrate slides followed by 30 min incubation with fibronectin at a concentration of 50 μg/mL. HUVEC were then seeded at 1.25×10⁶ cells/mL and incubated for 24 hrs. Slides were then washed by slowly passing PBS into the wells. Rhodamine-labeled leukosomes and liposomes, resuspended in EBM-2 media, were then infused into the slides using a Harvard Apparatus PHD 2000 Infusion syringe pump at a speed of 100 μL/min for 30 min. In order to investigate the mechanism of adhesion of leukosomes, either LFA-1 or CD45 were blocked on leukosome surface as indicated in the previous paragraph.

Evaluation of Protein Orientation into Leukosome Bilayer.

After infusion was complete, cells were briefly washed in PBS then fixed for 10 min using 4% paraformaldehyde at room temperature. Nuclei were then stained by infusing cells for 1 min with a PBS solution containing 4′,6-diamidino-2-phenylindole (DAPI), and washed to remove any free DAPI. Cells were then left in PBS and immediately images using an inverted Nikon Eclipse Ti fluorescence microscope equipped with a Hamamatsu ORCA-Flash 2.8 digital camera.

In Vivo Studies.

Ear inflammation was generated in Balb/c mice (Charles River Laboratories, Wilmington, Mass., USA) by a one-time injection of LPS (10 μg) in the right ear. Particles were administrated 30 min after LPS injection. Mice were prepared for intravital microscopy imaging at 1- and 24-hrs after particles injection to assess their targeting and distribution (vascular vs. extravascular space).

Histology of Ear Tissue.

Explanted Mice ears were washed twice with PBS and embedded in a cryomold in O.C.T. (Tissue-Tek® O.C.T. Compound, Sakura® Finetek), and instantly frozen at −80° C. Ten-μm-thick slides were obtained cutting ears block with a cryostat at −20° C. The slides were then stored at −20° C. until analysis.

For Masson's trichrome staining, slides were thawed and washed with xylene and ethanol solutions at different concentrations, and stained using Trichrome Stain after rehydration in distilled water, following the manufacturer protocol (Connective Tissue Stain Abcam®). Images were captured at 20× magnification with a Nikon Eclipse 80i microscope (Digital Sight DS-U3 camera).

Intravital Experiments. Imaging of Particle Accumulation in Ear.

Anesthetized animals were placed and imaged on an upright Nikon AIR MP-ready laser scanning intravital confocal microscopy (IVM) platform equipped with a resonance scanner, isoflurane anesthesia system, heated stage, and custom coverslip mounts. Before imaging, a bolus injection of 70 kDa FITC-dextran (50 μL in PBS) was used to delineate the vasculature. Images were obtained with a three-channel setup in which fluorescence was collected at 488/525 nm for FITC dextran, and at 561/579 nm for rhodamine-labeled particles. Image acquisition was performed over selected fields of view (FOVs) with resolution of 512×256 pixels with an optical slice thickness of 7.1 μm. Imaging to determine extent of leukosomes was performed 1- and 24-hrs after the initial i.v. injection (50 μL, 1 mg/mL).

Image Analyses and Particle Quantification. The average number of particles (leukosomes or liposomes) preferentially accumulated in ear microenvironment was enumerated in video stills using Nikon NIS element AR software (Nikon, Mellville, N.Y., USA). Select FOVs were chosen from time-lapse videos and automated object measurement feature was used to calculate area fraction fluorescent particles in each frame where a particle was defined by setting low and high pixel thresholds to include only visible red fluorescent particles and to exclude single noise pixels. The settings were applied to all frames and automated counting function used to generate average area fraction of particles for each time point. These settings were also kept constant across treatment groups. The average area fraction covered by fluorescent particles was normalized to the imaging area and then plotted as a function of time.

Biodistribution and Pharmacokinetic Profile of Leukosomes.

Balb/c mice were i.v. injected with 2 mg of rhodamine-labeled liposomes and leukosomes (n=5 for each group) in order to evaluate particle biodistribution and pharmacokinetic profile. After 24 hr, mice were sacrificed, and major organs (kidney, liver, kidney, and lung) and ear tissue were collected, washed twice with PBS, weighted and transferred in a falcon tube. Tubes were then filled with formamide (1 mL per 100 mg of tissue weight) and tissues were homogenized. After 2 hr's incubation at room temperature, samples were centrifuged at 5,000×g for 10 min, and the supernatant was collected and spectrofluorometrically analyzed for rhodamine detection (excitation/emission 561/579; slit width, 5 nm). Results were represented as relative signal per organ (%) based on a standard curve to calibrate rhodamine-labeled phospholipid.

For pharmacokinetic studies, blood was collected from the retro orbital plexus (n=5 for each group) at 24 hr after injection, and centrifuged at 1,500 rpm for 10 min to isolate plasma. Rhodamine concentration was measured based on fluorescence and calculated as aforementioned.

Bioluminescence Imaging of Lipopolysaccharide-Induced Acute Ear Inflammation.

Bioluminescence imaging of mice was used to confirm local inflammation. The right ears of mice were inflamed with a subcutaneous injection of 10 μL of LPS. Mice were imaged for bioluminescence (BLI) 5 min after i.p. administration of 5 mg (250-300 mg/kg) of luminol (Sigma-Aldrich) for 5 min at medium binning and an f/stop of 1 on an IVIS Spectrum. Luminol is a small molecule that enables noninvasive bioluminescence imaging of myeloperoxidase (Gross et al., 2009) (an enzyme found only in activated phagocytes, such as neutrophils). Images were analyzed using the Living Image Software and the average radiance in both ears was collected.

Statistical Analysis.

All data are presented as means 6 standard error of the mean (SEM). Intravital microscopy data are presented as means±standard error of the mean obtained from at least 10 FOVs for n=4. GraphPad statistical software (La Jolla, Calif., USA) was used to compute statistical significance between groups and control using student's t-test and one-way ANOVA test to compare differences between groups. A value of p=0.05 was considered statistically significant.

Results

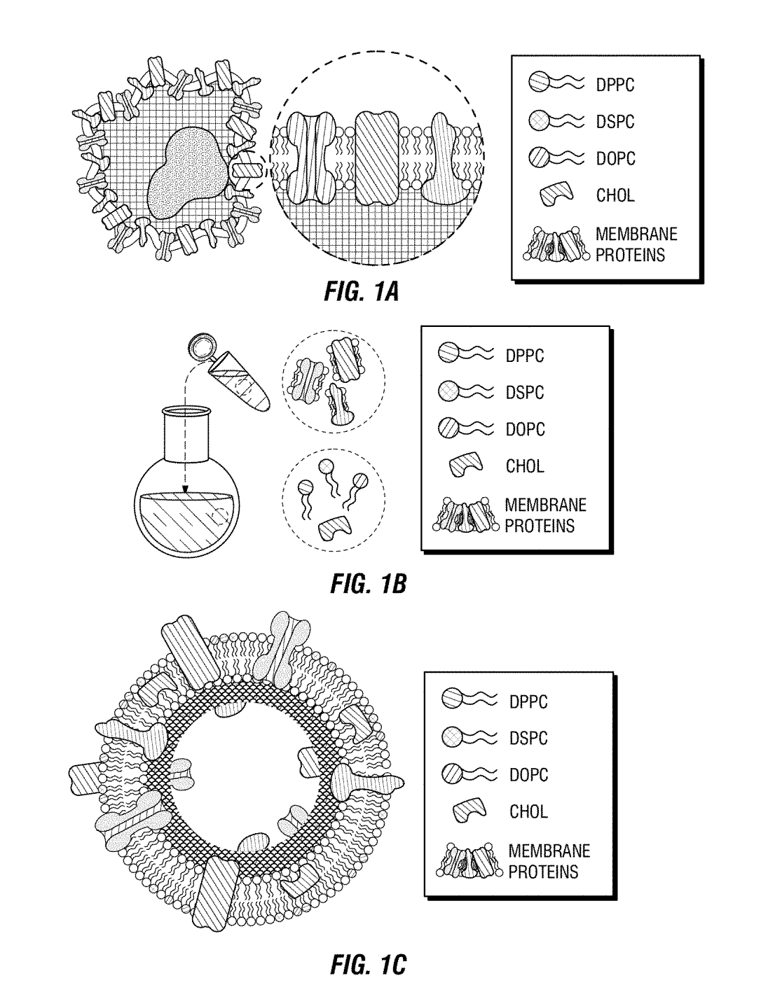

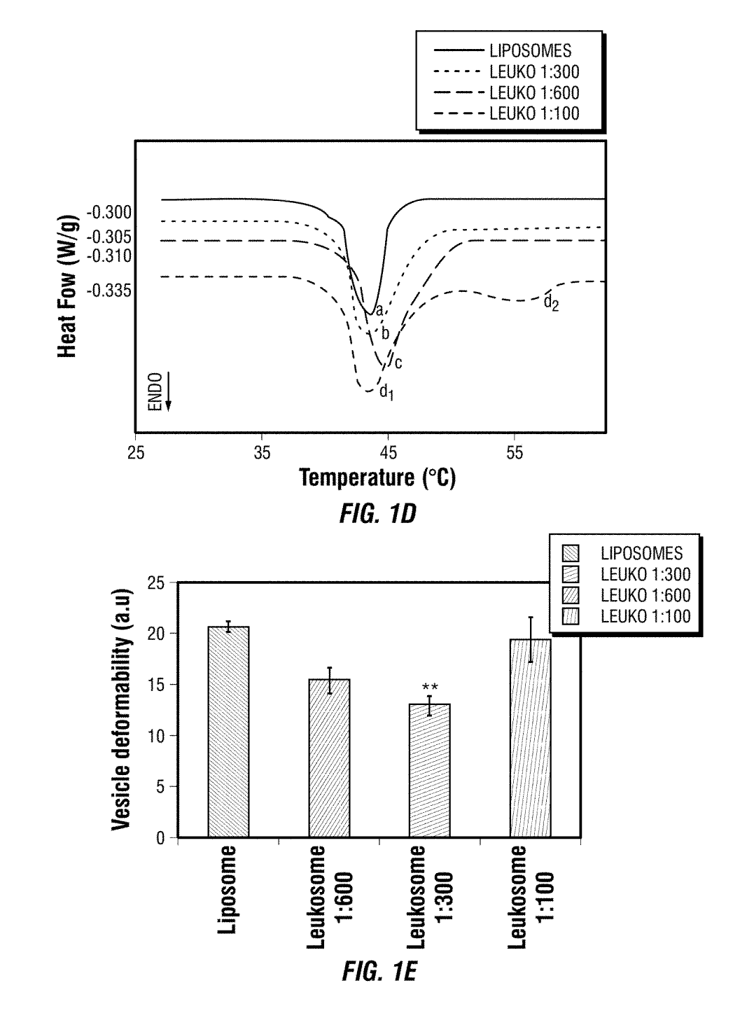

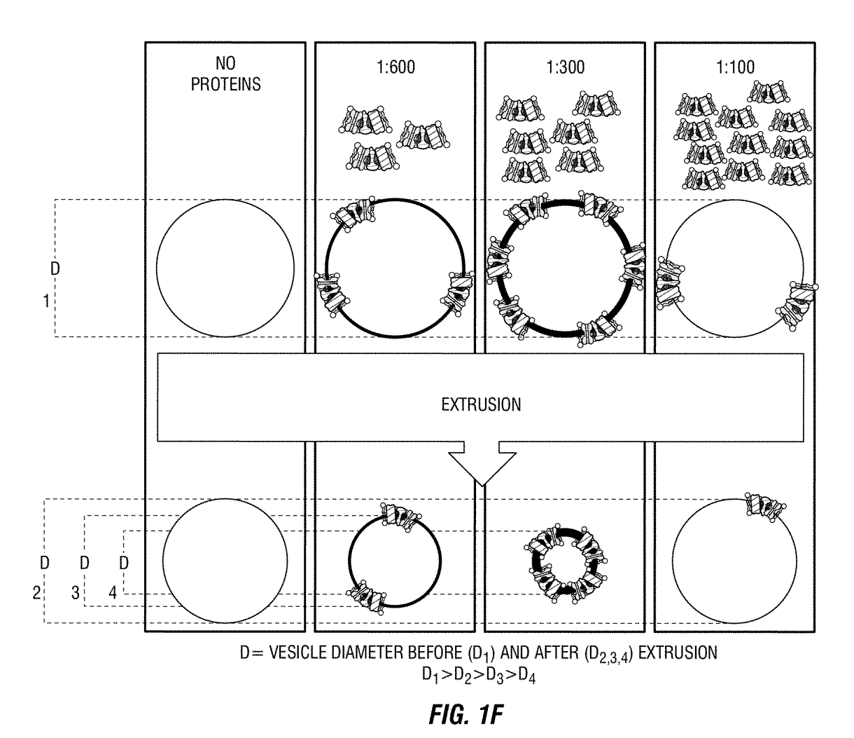

The membrane proteins that constitute the leukosome were extracted from both primary and immortalized immune cells. Once isolated, the material maintained the initial membrane protein content for up to one month when lyophilized and preserved at −20° C. or −80° C. A mixture of cholesterol and synthetic choline-based phospholipids (DPPC, DSPC and DOPC, see method section) was assembled that mimicked the physiologic composition of the plasmalemma (Bretscher, 1972), with the purified protein fraction using the established thin-layer evaporation (TLE) method (FIG. 1A, FIG. 1B, and FIG. 1C). Unilamellar vesicles were obtained by extrusion through cellulose acetate membranes (200-nm pore size) while unincorporated material was eliminated through dialysis. To hydrate the lipid film, three different weight-to-weight ratios of isolated cell membrane proteins-to-synthetic phospholipids were evaluated (1:100, 1:300 and 1:600). Differential scanning calorimetry (DSC) was used to investigate the bilayer transition temperature (Tₘ), which provides insight into the thermodynamic changes of the leukosome bilayer following the incorporation of membrane proteins (Demetzos, 2008). Compared to control liposomes (Tₘ=36.57° C.), the 1:300 ratio (Tₘ=40.76° C.) resulted in the highest incorporation of leukocyte membrane proteins, followed by the 1:600 (Tₘ=39.96° C.) and 1:100 ratios (Tₘ=36.85° C.) (FIG. 1D). The data suggested that the degree of protein integration within the lipid bilayer correlated to the increase of Tₘ, possibly due to a packing effect of the leukosome bilayer. However, at the higher ratio (1:100), a Tₘ was measured similar to control liposomes, suggesting the existence of a threshold above which leukosome bilayers could not be further enriched with protein content.

As inferred by the Tₘ of 55° C. in the thermogram of FIG. 1D, this phenomenon was likely due to the formation of protein aggregates because of the heating and vortexing steps during the TLE procedure. This was further confirmed by the extrusion assay (Manconi et al., 2011). Briefly, this assay is based on the extrusion of lipid formulations through membranes with 50-nm pore, such that a slight decrease in the vesicles' diameter indicates a higher deformability of the bilayer (Mura et al., 2009) (FIG. 1E). Here, the decrease in the leukosomes' diameter correlated with the increase of protein content into the lipid bilayer (from 1:100 and 1:600 up to 1:300 protein-to-lipid ratio, FIG. 1F). Taken together these results indicated that the 1:300 ratio provided the best compromise between stability, protein content and membrane fluidity, and was chosen for the assembly of the leukosomes in all subsequent studies.

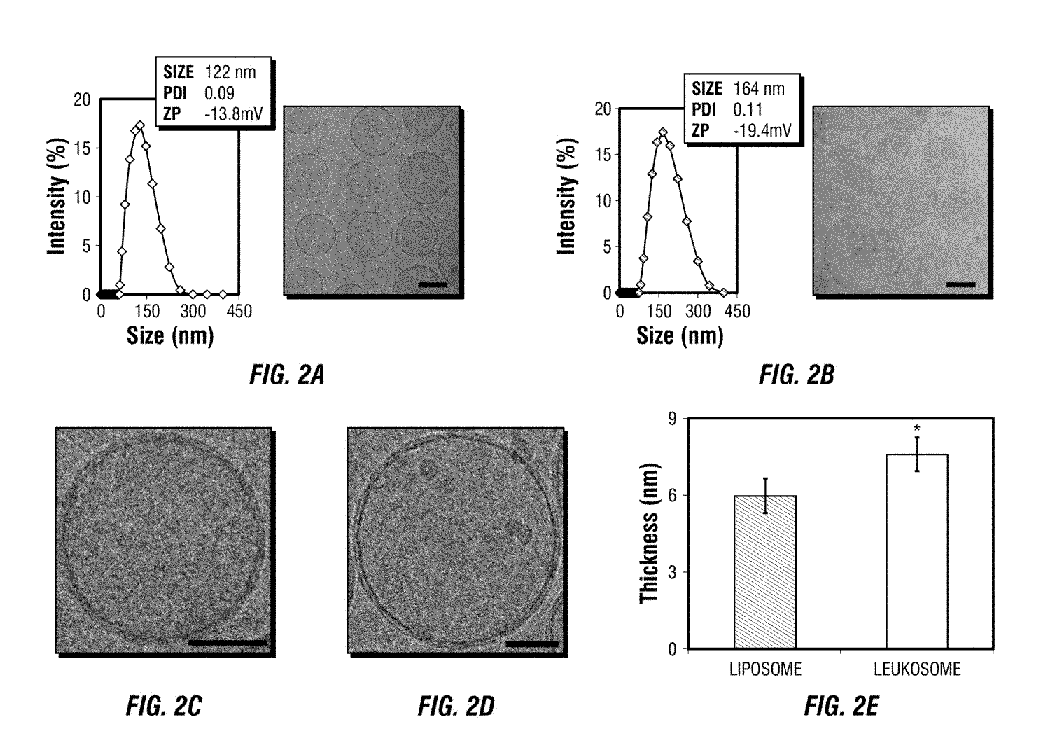

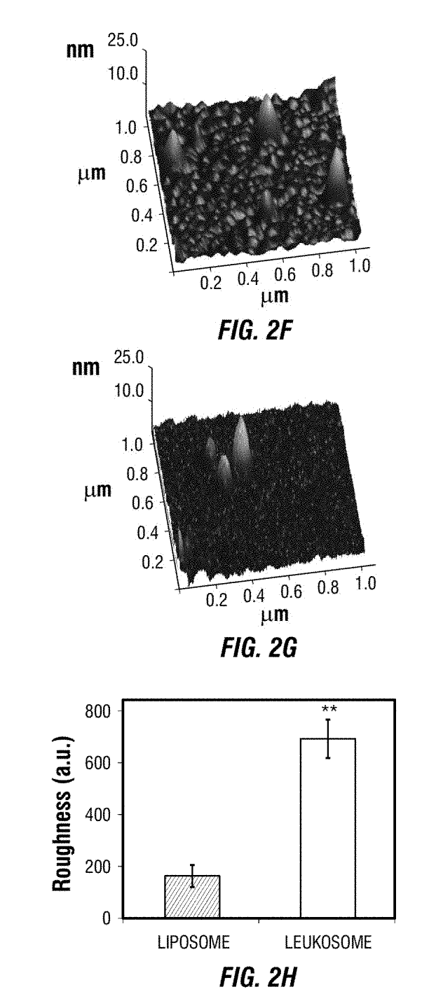

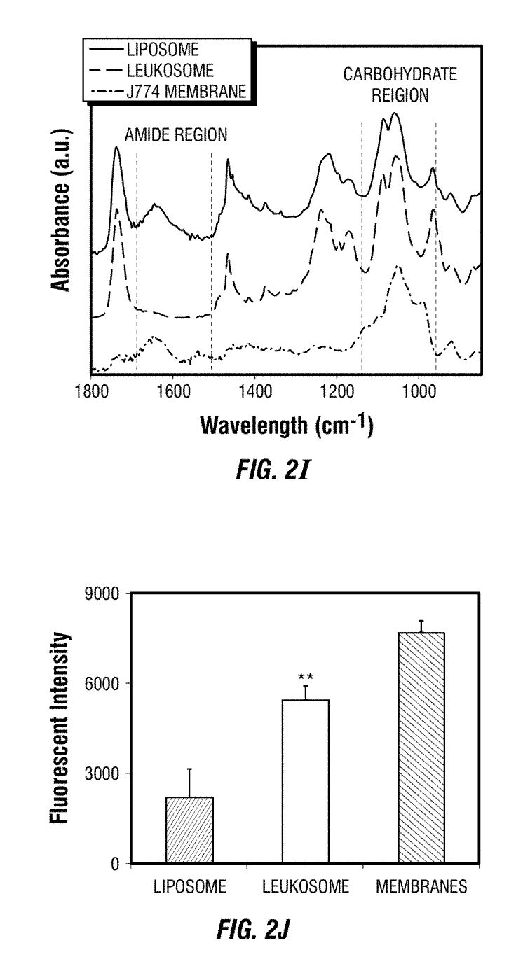

After extrusion and dialysis, dynamic light scattering (DLS), zeta potential, and cryo-TEM analyses were used to evaluate the size, homogeneity, surface charge, shape, and structure of leukosomes (FIG. 2A, FIG. 2B, FIG. 2C, FIG. 2D, FIG. 2E, FIG. 2F, FIG. 2G, FIG. 2H, FIG. 2I, and FIG. 2J). The biomimetic formulation of the proteolipid material produced leukosomes with homogeneous size (˜120 nm, with >90% unilamellar vesicles for both samples), as demonstrated by low magnification cryo-TEM and the polydispersity index (PDI) from DLS (FIG. 2A and FIG. 2B). Compared to liposomes, the less negative surface charge of leukosomes (−19.4 mV vs. −13.8 mV, respectively) was attributed to the shielding effect of the membrane proteins toward the negative charge of the lipid phosphate groups. High magnification cryo-TEM revealed a 1.3-fold increase in bilayer thickness compared to control liposomes (FIG. 2C, FIG. 2D and FIG. 2E). Corresponding line profiles through lipid bilayers of both vesicles were selected from cryo-TEM images with similar defocus values to ensure comparable imaging conditions.

Topographical analysis by atomic force microscopy (AFM) confirmed the increased surface roughness of leukosomes suggesting the presence of hinged structures in their bilayer (Chow, 2007) (FIG. 2F, FIG. 2G, and FIG. 2H). In addition, viscoelastic properties of both liposomes and leukosomes were investigated by calculating the Young's modulus, where an increase corresponds to a higher stiffness of the material (Schaap et al., 2012). The elastic modulus for leukosomes demonstrated a slight, yet significant (p<0.05), increase in stiffness when compared to liposomes (476 kPa and 423 kPa, respectively). Next, the vibrational modes and chemical signatures of leukocyte membranes (black line), leukosomes (red) and liposomes (green) were identified through Fourier transform infrared (FTIR) spectroscopy (FIG. 2I). Three protein absorption bands were present: the amide I band (1700-1600 cm⁻¹) due to the C═O stretching vibrations, amide II (1580-1510 cm⁻¹) associated with the N—H bending with a contribution of the C—N stretching vibrations, and amide III (1400-1200 cm⁻¹) due to the N—H bending and stretching vibrations from C—Cα and C—N.

In addition, the 1200-900 cm⁻¹ region showed the absorption of protein-associated sugar chains suggesting the presence of glycosylated proteins in the membranes (Mereghetti et al., 2014). To confirm the glycosylation of the proteolipid material, leukosomes with wheat germ agglutinin (WGA), a lectin that selectively binds N-acetyl-D-glucosamine and glycosylated sialic acid residues. Spectrofluorometric analysis verified the presence of glycosylated proteins on the surface of the leukosome, showing the integration, correct orientation, and stabilization of membrane proteins in their post-transcriptionally modified state⁷ (FIG. 2J).

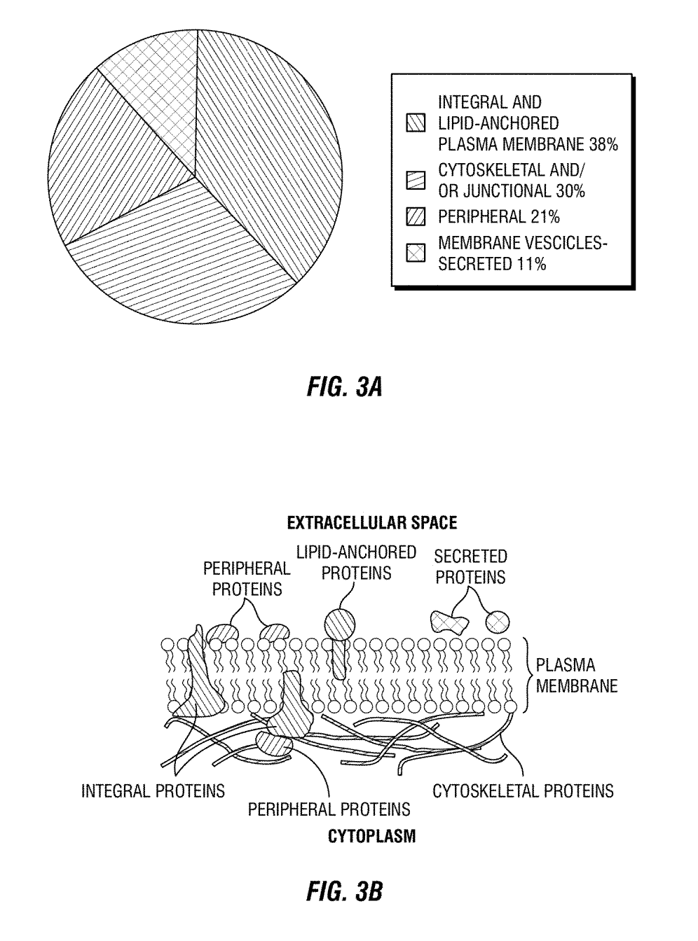

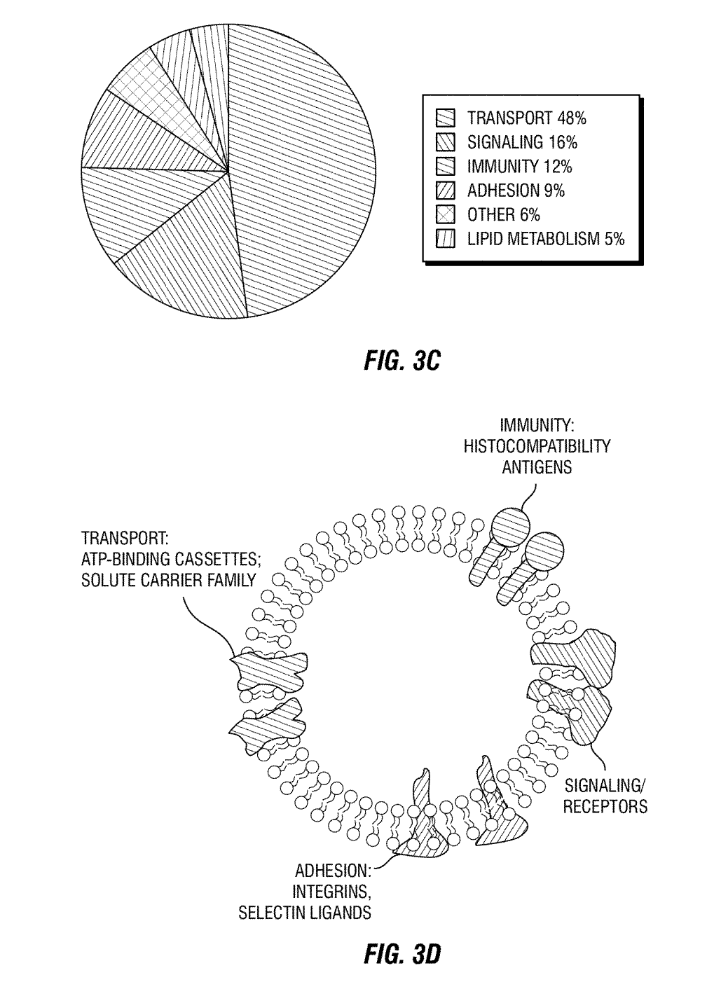

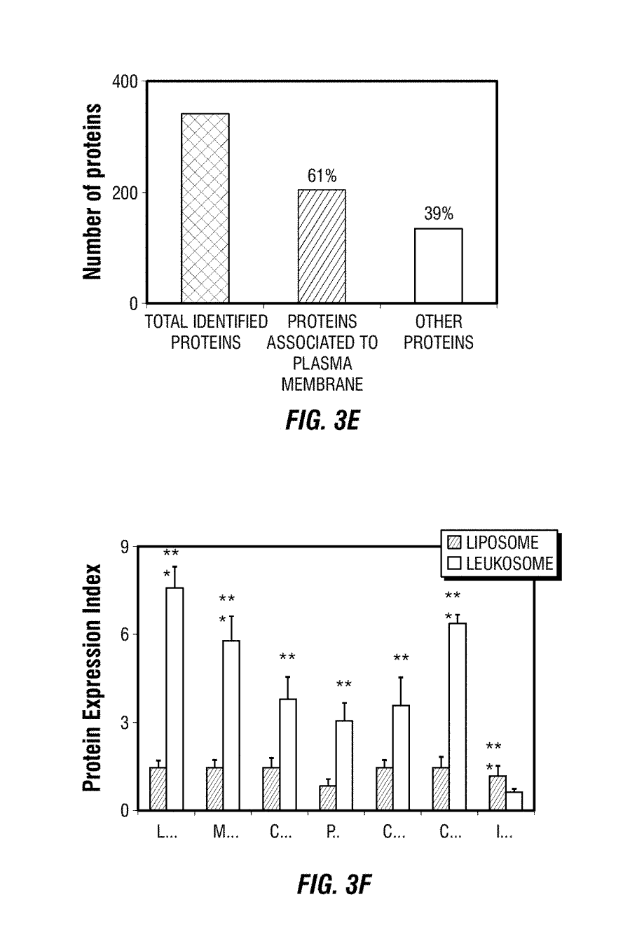

The proteomic profiling of the leukosome resulted in the identification of 342 distinct proteins (Table 2). Two thirds were small proteins (10-50 KDa) and more than half of the total identified with high confidence scores (300-2000) with a sequence coverage ranging from 10% to 30%. Leukosome proteins were classified in the following manner: integral or lipid-anchored to plasma membrane (38%), cytoskeletal and/or junctional (30%), peripheral (21%), and vesicular or secreted proteins (11%) (FIG. 3A and FIG. 3B). The presence of proteins from other cellular compartments (primarily ribosomes and mitochondria) was in line with results found in the literature (Durr et al., 2004; Lund et al., 2009; Liu et al., 2010; Liang et al., 2006; Corbo et al., 2014) and was attributed to the dynamic trafficking of proteins between internal organelles and the cell surface (Lodish et al., 2000; Benmerah et al., 2003). A functional classification revealed proteins involved in transport (48%), signaling (16%), immunity (12%), cell adhesion (9%), lipid metabolism (5%), and structure (4%) (FIG. 3C and FIG. 3D). As can be observed, the majority of the identified proteins were associated to the leukocyte plasma membrane (FIG. 3E).

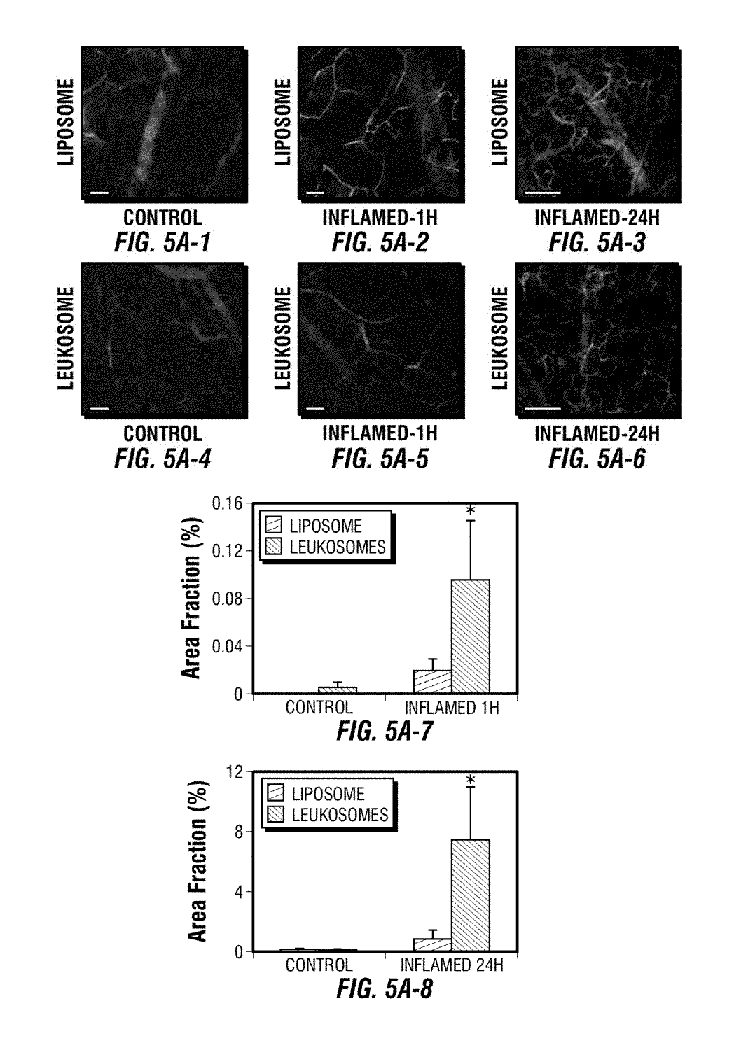

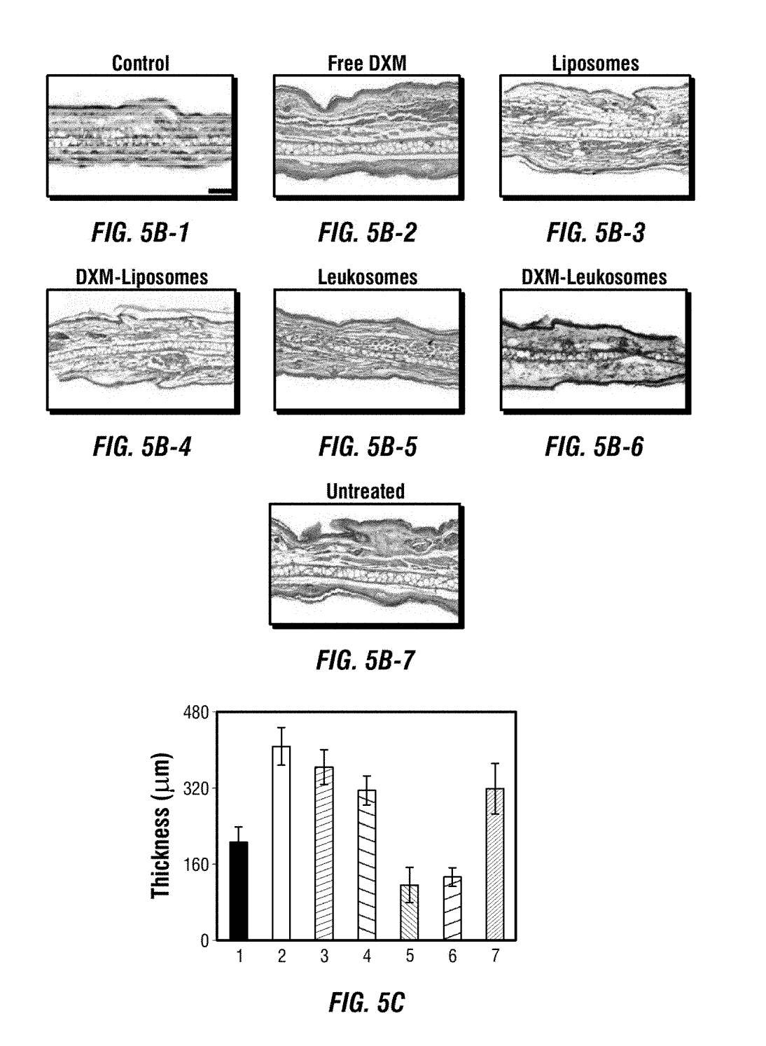

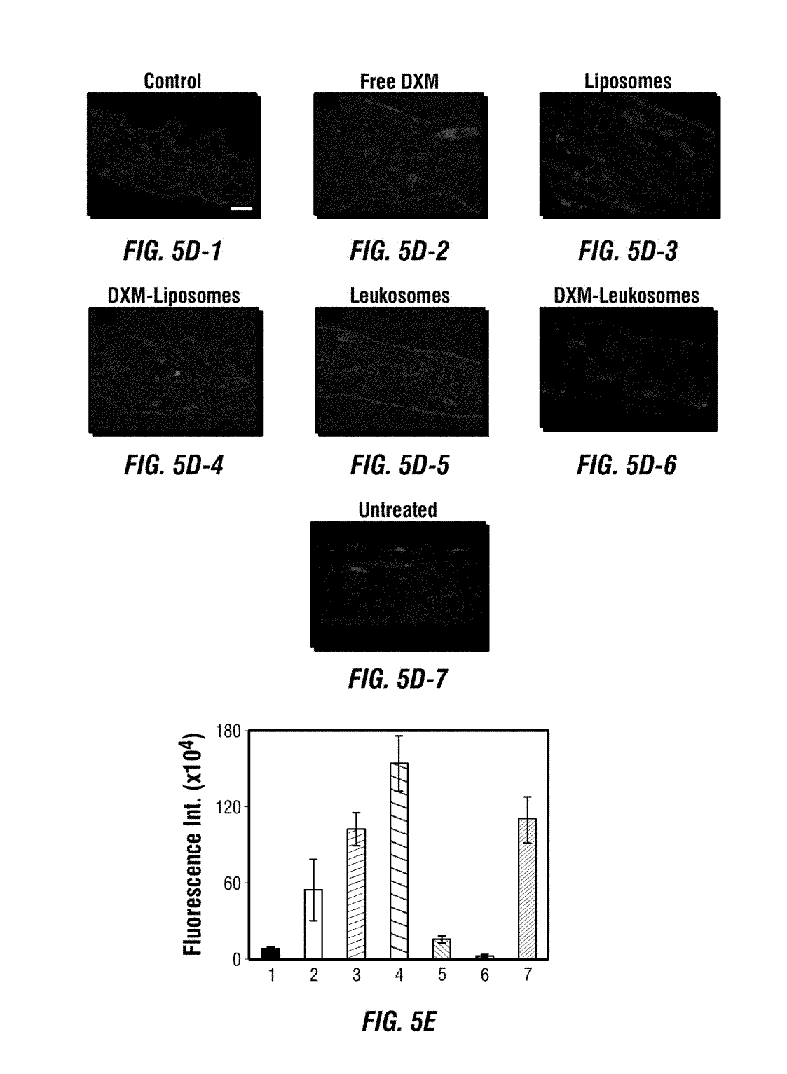

The presence of critical leukocyte surface proteins such as those involved in leukocyte adhesion to inflamed endothelium (e.g., leukocyte function-associated antigen (LFA) 1, macrophage antigen (MAC) 1), and in self-tolerance (e.g., leukocyte common antigen CD45) were confirmed on the surface of the donor cell through fluorescence microscopy and flow cytometry (FIG. 5A and FIG. 5B). In addition to these general markers, P-selectin glycoprotein ligand 1 (PSGL-1), and the ‘marker-of-self’ CD47 have a fundamental role in leukocyte firm adhesion on any substrate that expresses P-selectin (Zarbock et al., 2011) (e.g., platelets and endothelial cells) and self-recognition (Soto-Pantoja et al., 2013), respectively. Immunolabeling with antibodies directed against the extracellular domain of these proteins qualitatively confirmed their presence and correct orientation within the leukosome's bilayer (FIG. 3F). In addition, fluorescently labeled antibodies versus LFA-1, Mac-1, PSGL-1, CD18, CD45, and CD47, were prepared as standards, and were then incubated separately with liposomes and leukosomes. Theoretical calculations (Hu et al., 2013) revealed a surface density of approximately 206, 149, 85, 144, 109, and 187 copies per μm² of LFA-1, Mac-1, PSGL-1, CD18, CD45, and CD47, respectively (Table 4).

Taken together, these data confirmed the successful transfer of leukocyte membrane-based markers on the surface of leukosomes in an amount sufficient to exert their activity (Robbins et al., 2010; Hu et al., 2013).

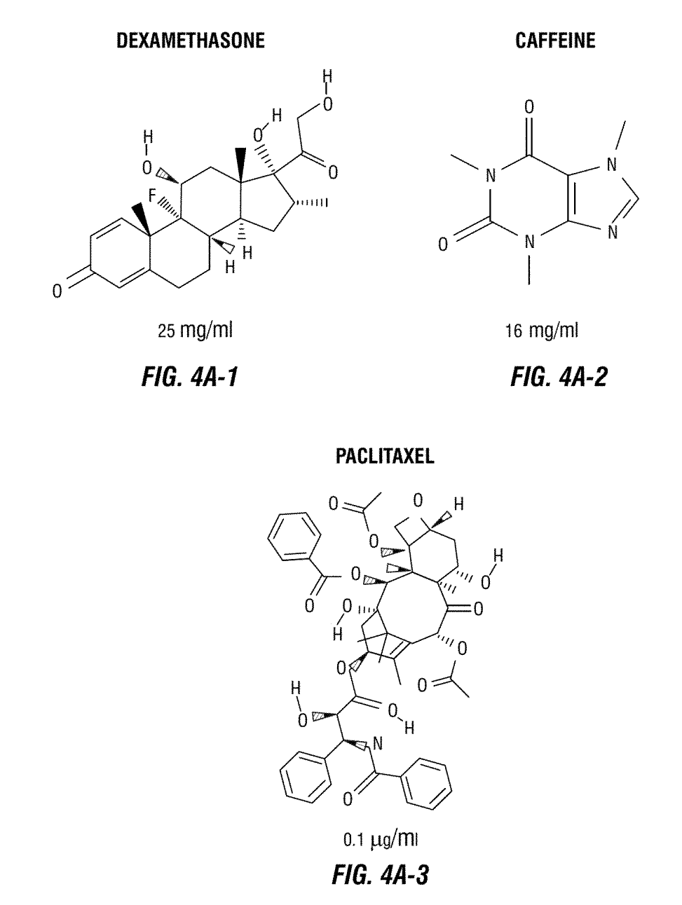

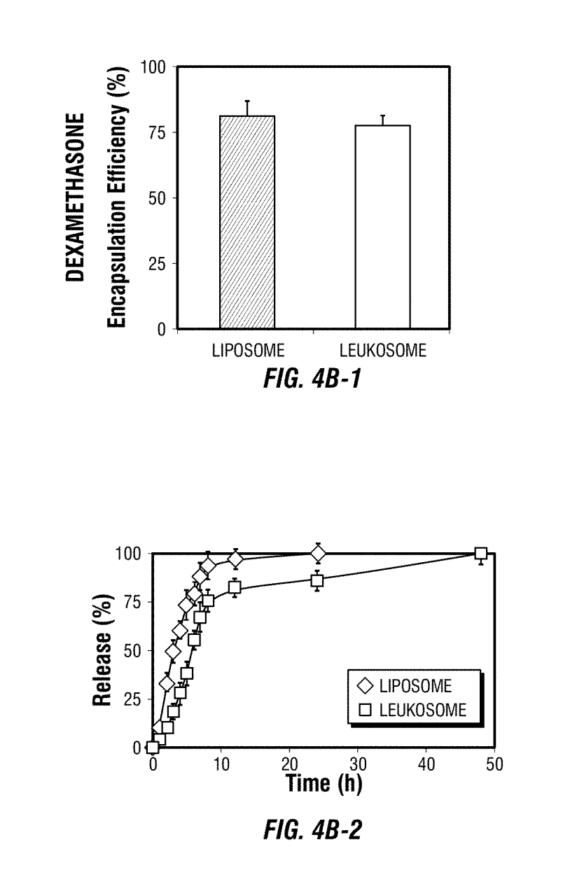

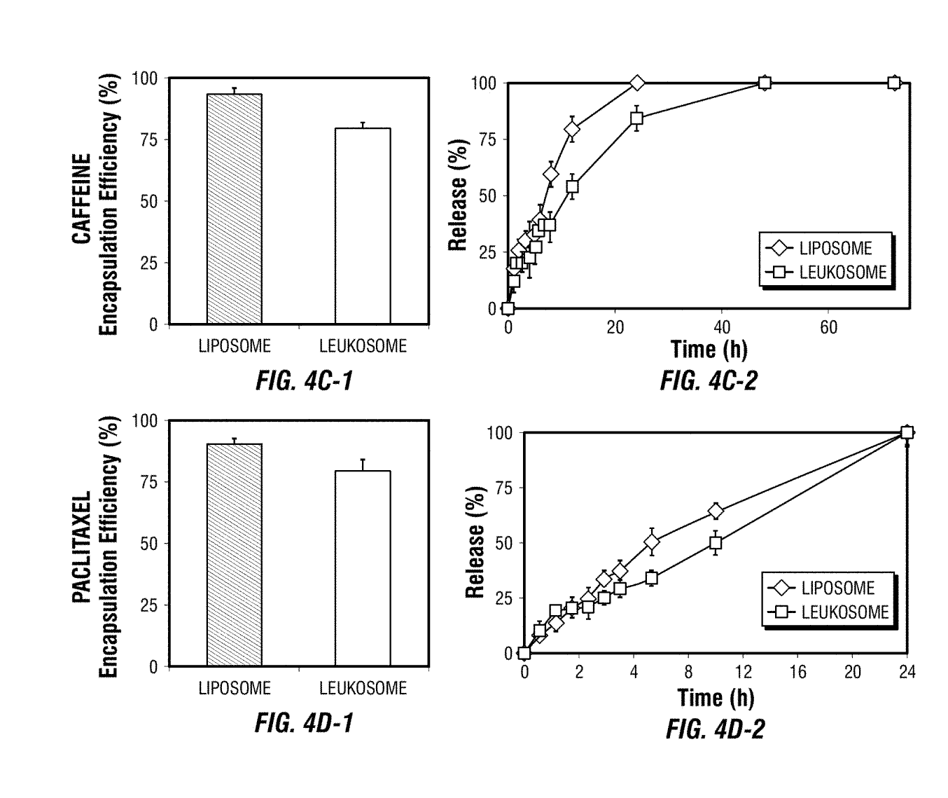

From the pharmaceutical standpoint, leukosomes showed similar stability after storage at 4° C. to conventional liposomes. DLS analysis revealed that empty leukosomes were stable for two weeks, with no significant change in vesicle size. After three weeks a slight increase (<20%) in leukosome diameter was observed but yielded no significant increase in their PDI. Leukosomes also retained similar loading and release properties of liposomes as well as their versatility for encapsulating compounds with various solubilities (FIG. 4A-1, FIG. 4A-2, FIG. 4A-3). Dexamethasone (DXM), caffeine, and paclitaxel were chosen as model compounds to represent small molecules with hydrophilic, amphiphilic, and hydrophobic characteristics, respectively (FIG. 4B-1, FIG. 4B-2, FIG. 4C-1, FIG. 4C-2, FIG. 4D-1, and FIG. 4D-2). Drug encapsulation did not significantly affect the physical features (e.g., size and polydispersity) with respect to empty leukosomes. However, a different effect on the surface charge was observed upon drug loading. While DXM and caffeine encapsulation produced a minimal change in surface charge, paclitaxel exhibited a more pronounced effect. Paclitaxel, in fact, increased the leukosomes' surface charge to positive values (15 mV). As previously shown for other hydrophobic drugs (Cosco et al., 2012), paclitaxel intercalates among the hydrophobic tails of the lipid bilayer which likely induced a structural rearrangement of the membrane (Bernsdorff et al., 1999), possibly through the exposure of the choline groups to the outer bilayer surface. The slight delay in the release of the different payloads from leukosomes (FIG. 4B-1, FIG. 4B-2, FIG. 4C-1, FIG. 4C-2, FIG. 4D-1, and FIG. 4D-2) could be attributed to the presence of the membrane proteins and to the increased bilayer thickness. A first-order kinetic release profile was observed for DXM and caffeine (FIG. 4B-1, FIG. 4B-2, FIG. 4C-1, FIG. 4C-2), while paclitaxel was released with zero-order kinetics (FIG. 4D-1 and FIG. 4D-2).

TABLE 2
Proteins Identidified in Leukosomes

| Uniprot | | | | | | % Seq |
|---|---|---|---|---|---|---|
| Accession | Name | Score | MW (Da) | Products | Peptides | Cov. |
| P50516 | ATPase, H+ transporting, lysosomal V1 subunit A | 506 | 68326 | 62 | 53 | 21.23 |
| Q9CR51 | ATPase, H+ transporting, lysosomal V1 subunit G1 | 748 | 13724 | 19 | 15 | 38.14 |
| Q8VDN2 | ATPase, Na+/K+ transporting, alpha 1 polypeptide | 1175 | 112983 | 136 | 72 | 25.22 |
| Q6PIE5 | ATPase, Na+/K+ transporting, alpha 2 polypeptide | 835 | 112218 | 90 | 77 | 15.49 |
| Q6PIC6 | ATPase, Na+/K+ transporting, alpha 3 polypeptide | 842 | 111692 | 89 | 66 | 18.07 |
| P18572 | Basigin | 418 | 42445 | 18 | 26 | 16.45 |
| P10810 | CD14 antigen | 659 | 39204 | 25 | 21 | 10.11 |
| Q62192 | CD180 antigen (Lymphocyte antigen 78) | 297 | 74303 | 27 | 36 | 8.02 |
| P15379 | CD44 antigen | 789 | 85617 | 25 | 37 | 6.56 |
| P31996 | CD68 antigen | 673 | 34818 | 35 | 16 | 8.9 |
| P10852 | CD98 | 1725 | 58337 | 90 | 43 | 30.61 |
| O89053 | Coronin | 242 | 50989 | 28 | 33 | 10.2 |
| Q61543 | E-selectin ligand 1 | 175 | 133734 | 42 | 94 | 2.81 |
| Q3U7R1 | Extended synaptotagmin 1 OS Mus musculus GN Esyt1 PE 2 SV | 190 | 121554 | 23 | 85 | 2.66 |
| | 2 | | | | | |
| P08101 | Fc receptor, IgG, low affinity IIb | 942 | 36695 | 32 | 22 | 13.33 |
| P08508 | Fc receptor, IgG, low affinity III | 449 | 30036 | 17 | 18 | 28.74 |
| P08752 | Guanine nucleotide binding protein (G protein), alpha inhibiting 2 | 1686 | 40489 | 50 | 29 | 29.58 |
| Q9DC51 | Guanine nucleotide binding protein (G protein), alpha inhibiting 3 | 233 | 40538 | 18 | 29 | 10.45 |
| P62874 | Guanine nucleotide binding protein (G protein), beta 1 | 341 | 37377 | 23 | 21 | 19.12 |
| P62880 | Guanine nucleotide binding protein (G protein), beta 2 | 387 | 37331 | 28 | 21 | 15.59 |
| P68040 | Guanine nucleotide binding protein (G protein), beta polypeptide | 966 | 35077 | 72 | 27 | 25.87 |
| | 2 like 1 | | | | | |
| P01899 | H-2 class I histocompatibility antigen, D-B alpha chain | 2343 | 40836 | 41 | 29 | 36.19 |
| P01897 | H-2 class I histocompatibility antigen, L-D alpha chain | 2333 | 40711 | 34 | 26 | 27.35 |
| P01900 | H-2 class I histocompatibility antigen, D-D alpha chain | 1283 | 41111 | 45 | 30 | 24.66 |
| P14427 | H-2 class I histocompatibility antigen, D-P alpha chain | 420 | 41342 | 24 | 28 | 11.41 |
| P01902 | H-2 class I histocompatibility antigen, K-D alpha chain | 791 | 41490 | 26 | 29 | 17.93 |
| P14429 | H-2 class I histocompatibility antigen, Q7 alpha chain | 621 | 37924 | 12 | 27 | 15.27 |
| P05555 | Integrin alpha M MAC-1 | 389 | 127481 | 76 | 77 | 13.18 |
| P09055 | Integrin beta 1 (fibronectin receptor beta) VLA-4 | 174 | 88232 | 24 | 55 | 10.4 |
| P11835 | Integrin beta 2 LFA-1 | 414 | 85026 | 58 | 68 | 20.88 |
| Q9CQW9 | Interferon induced transmembrane protein 3 OS Mus musculus | 4422 | 14954 | 36 | 9 | 32.85 |
| | GN Ifitm3 PE 1 SV 1 | | | | | |
| Q8C129 | Leucyl/cystinyl aminopeptidase | 113 | 117304 | 29 | 68 | 6.54 |
| A1L314 | Macrophage expressed gene 1 proteinM | 2977 | 78391 | 112 | 42 | 25.53 |
| Q99LR1 | Monoacylglycerol lipase ABHD12 OS Mus musculus GN | 419 | 45270 | 22 | 29 | 8.79 |
| | Abhd12 PE 1 SV 2 | | | | | |
| O35682 | Myeloid associated differentiation marker OS Mus musculus | 256 | 35285 | 15 | 13 | 10.31 |
| | GN Myadm PE 2 SV 2 | | | | | |
| Q8BLF1 | Neutral cholesterol ester hydrolase 1 OS Mus musculus GN | 301 | 45740 | 29 | 23 | 23.53 |
| | Nceh1 PE 1 SV 1 | | | | | |
| P57716 | Nicastrin | 101 | 78492 | 24 | 44 | 6.21 |
| Q8BG07 | Phospholipase D4 OS Mus musculus GN Pld4 PE 2 SV 1 | 346 | 56154 | 22 | 29 | 8.55 |
| P06800 | Protein tyrosine phosphatase, receptor type, C | 227 | 144605 | 51 | 83 | 9.22 |
| P61027 | RAB10, member RAS oncogene family | 3626 | 22541 | 34 | 18 | 29 |
| P46638 | RAB11B, member RAS oncogene family | 1283 | 24490 | 40 | 18 | 35.78 |
| Q9DD03 | RAB13, member RAS oncogene family | 1082 | 22770 | 12 | 17 | 18.81 |
| Q91V41 | RAB14, member RAS oncogene family | 3984 | 23897 | 52 | 21 | 57.21 |
| Q8K386 | RAB15, member RAS oncogene family | 1280 | 24319 | 21 | 21 | 29.72 |
| Q9D1G1 | RAB1B, member RAS oncogene family | 4716 | 22187 | 53 | 18 | 35.32 |
| Q504M8 | RAB26, member RAS oncogene family | 2462 | 28619 | 14 | 19 | 9.62 |
| P59279 | RAB2B, member RAS oncogene family | 619 | 24198 | 26 | 23 | 44.44 |
| Q923S9 | RAB30, member RAS oncogene family | 2449 | 23058 | 18 | 20 | 27.59 |
| Q6PHN9 | RAB35, member RAS oncogene family | 2491 | 23025 | 16 | 16 | 16.92 |
| Q8BHC1 | RAB39B, member RAS oncogene family | 2460 | 24636 | 29 | 22 | 32.86 |
| P63011 | RAB3A, member RAS oncogene family | 2454 | 24970 | 16 | 16 | 8.64 |
| Q9CZT8 | RAB3B, member RAS oncogene family | 2463 | 24757 | 16 | 16 | 14.16 |
| P35276 | RAB3D, member RAS oncogene family | 2469 | 24416 | 15 | 18 | 17.35 |
| Q8CG50 | RAB43, member RAS oncogene family | 2796 | 23263 | 25 | 25 | 35.24 |
| Q91ZR1 | RAB4B, member RAS oncogene family | 2462 | 23629 | 19 | 20 | 26.29 |
| P61021 | RAB5B, member RAS oncogene family | 1069 | 23707 | 30 | 14 | 49.3 |
| P35278 | RAB5C, member RAS oncogene family | 1512 | 23413 | 52 | 14 | 51.39 |
| P55258 | RAB8A, member RAS oncogene family | 3685 | 23668 | 39 | 17 | 47.34 |
| P61028 | RAB8B, member RAS oncogene family | 3558 | 23603 | 36 | 15 | 31.4 |
| P61226 | RAP2B, member of RAS oncogene family | 847 | 20504 | 31 | 17 | 38.25 |
| Q9QUI0 | ras homolog gene family, member A | 1333 | 21782 | 33 | 16 | 26.42 |
| P84096 | ras homolog gene family, member G | 578 | 21309 | 28 | 17 | 28.8 |
| Q99JI6 | RAS related protein 1b; similar to GTP-binding protein (smg | 6718 | 20825 | 68 | 15 | 40.76 |
| | p21B) | | | | | |
| P35283 | Ras related protein Rab 12 OS Mus musculus GN Rab12 PE 1 SV | 2460 | 27329 | 20 | 26 | 16.87 |
| | 3 | | | | | |
| P56371 | Ras related protein Rab 4A OS Mus musculus GN Rab4a PE 1 SV | 2482 | 24409 | 17 | 23 | 18.81 |
| | 2 | | | | | |
| P51150 | Ras related protein Rab 7a OS Mus musculus GN Rab7a PE 1 SV | 8962 | 23490 | 109 | 20 | 63.29 |
| | 2 | | | | | |
| P62835 | RAS-related protein-1a | 2720 | 20987 | 27 | 15 | 33.7 |
| P14206 | Laminin receptor 1 | 1090 | 32838 | 25 | 25 | 21.36 |
| Q9WV27 | Sodium potassium transporting ATPase subunit alpha 4 OS Mus | 151 | 114887 | 49 | 71 | 10.27 |
| | musculus GN Atp1a4 PE 1 SV 3 | | | | | |
| P97370 | Sodium potassium transporting ATPase subunit beta 3 OS Mus | 1302 | 31776 | 26 | 20 | 17.27 |
| | musculus GN Atp1b3 PE 1 SV 1 | | | | | |
| P31650 | Solute carrier family 6 (neurotransmitter transporter, GABA), | 106 | 69961 | 11 | 30 | 3.99 |
| | member 11 | | | | | |
| Q9R1J0 | Sterol 4 alpha carboxylate 3 dehydrogenase decarboxylating OS | 527 | 40686 | 31 | 28 | 36.19 |
| | Mus musculus GN Nsdhl PE 2 SV 1 | | | | | |
| P54116 | Stomatin | 5278 | 31375 | 73 | 23 | 50.7 |
| Q9WU81 | Sugar phosphate exchanger 2 OS Mus musculus GN Slc37a2 PE 2 | 190 | 55073 | 21 | 26 | 13.77 |
| | SV 1 | | | | | |
| Q80X71 | Transmembrane protein 106B OS Mus musculus GN Tmem106b | 193 | 31172 | 21 | 17 | 12 |
| | PE 2 SV 1 | | | | | |
| Q6ZQM8 | UDP glycosyltransferase 1 family polypeptide A13 | 438 | 59758 | 45 | 34 | 16.95 |
| Q60932 | voltage-dependent anion channel 1 | 3309 | 32352 | 81 | 21 | 45.95 |
| Cytoskeletal | | | | | | |
| and/or | | | | | | |
| junctional | | | | | | |
| proteins | | | | | | |
| P40124 | CAP, adenylate cyclase-associated protein 1 | 115 | 51565 | 8 | 41 | 2.11 |
| P61161 | ARP2 actin-related protein 2 homolog | 843 | 44761 | 24 | 23 | 9.9 |
| Q99JY9 | ARP3 actin-related protein 3 homolog | 1684 | 47357 | 61 | 39 | 33.97 |
| P08101 | Fc receptor, IgG, low affinity IIb | 942 | 36695 | 32 | 22 | 13.33 |
| Q9CVB6 | Actin related protein 2/3 complex, subunit 2 | 420 | 34357 | 36 | 37 | 20.33 |
| Q9WV32 | Actin related protein 2/3 complex, subunit 1B | 282 | 41064 | 12 | 28 | 11.29 |
| Q9JM76 | Actin related protein 2/3 complex, subunit 3 | 908 | 20525 | 19 | 17 | 25.28 |
| P59999 | Actin related protein 2/3 complex, subunit 4 | 2006 | 19667 | 33 | 15 | 22.62 |
| P68134 | Actin, alpha 1, skeletal muscle | 5883 | 42051 | 165 | 34 | 41.64 |
| P60710 | actin, beta | 19249 | 41737 | 344 | 34 | 65.07 |
| Q8BFZ3 | Actin, beta-like 2 | 4570 | 42004 | 85 | 35 | 34.04 |
| P63268 | Actin, gamma 2, smooth muscle, enteric | 5858 | 41877 | 163 | 33 | 34.31 |
| P18760 | Cofilin 1, non-muscle; similar to Cofilin-1 (Cofilin, non-muscle | 2049 | 18560 | 69 | 19 | 45.78 |
| | isoform) | | | | | |
| P45591 | cofilin 2, muscle | 843 | 18710 | 19 | 18 | 13.86 |
| O89053 | Coronin, actin binding protein 1A | 242 | 50989 | 28 | 33 | 10.2 |
| P26040 | Ezrin | 878 | 69407 | 102 | 72 | 14.51 |
| P04104 | Keratin 1 | 3175 | 65606 | 112 | 47 | 23.55 |
| P02535 | Keratin 10 | 1375 | 57770 | 66 | 38 | 15.96 |
| Q61414 | keratin 15 | 310 | 49138 | 27 | 42 | 17.04 |
| Q9QWL7 | keratin 17 | 641 | 48162 | 66 | 41 | 25.17 |
| Q3TTY5 | keratin 2 | 519 | 70923 | 89 | 55 | 8.35 |
| Q922U2 | keratin 5 | 2196 | 61767 | 103 | 43 | 19.66 |
| Q9Z331 | keratin 6B | 1461 | 60322 | 68 | 42 | 13.7 |
| Q9R0H5 | keratin 71 | 1879 | 57383 | 35 | 45 | 11.83 |
| Q6NXH9 | keratin 73 | 1914 | 58912 | 46 | 46 | 11.5 |
| Q6IFZ9 | keratin 74 | 1891 | 54747 | 35 | 46 | 14.75 |
| Q8BGZ7 | keratin 75 | 1284 | 59741 | 80 | 45 | 28.68 |
| Q3UV17 | keratin 76 | 972 | 62845 | 51 | 47 | 26.09 |
| Q6IFZ6 | keratin 77 | 1871 | 61359 | 58 | 47 | 11.36 |
| Q61233 | lymphocyte cytosolic protein 1/plastilin | 2845 | 70149 | 198 | 54 | 45.14 |
| P26041 | moesin | 2134 | 67767 | 190 | 64 | 43.5 |
| Q99K51 | Plastin 3 OS Mus musculus GN Pls3 PE 1 SV 3 | 744 | 70742 | 60 | 56 | 20.63 |
| P05213 | tubulin, alpha 1B; | 6366 | 50152 | 115 | 34 | 27.94 |
| P05214 | tubulin, alpha 3A | 4799 | 49960 | 101 | 34 | 21.33 |
| Q9CVB6 | actin related protein 2/3 complex, subunit 2 | 420 | 34357 | 36 | 37 | 20.33 |
| Q99JB2 | Stomatin (Epb7.2)-like 2 | 452 | 38385 | 30 | 29 | 30.03 |
| Q61937 | nucleophosmin 1 | 5722 | 32560 | 83 | 22 | 14.04 |
| P68369 | tubulin, alpha 1A | 4914 | 50136 | 110 | 34 | 27.94 |
| P84078 | ADP ribosylation factor 1 OS Mus musculus GN Arf1 PE 1 SV 2 | 472 | 20697 | 24 | 14 | 32.6 |
| P62962 | profilin 1 | 454 | 14957 | 26 | 16 | 55 |
| P80316 | T complex protein 1 subunit epsilon OS Mus musculus GN Cct5 | 176 | 59624 | 20 | 45 | 10.54 |
| | PE 1 SV 1 | | | | | |
| P62082 | 40S ribosomal protein S7 OS Mus musculus GN Rps7 PE 2 SV 1 | 723 | 22127 | 27 | 21 | 29.38 |
| P26043 | radixin | 1073 | 68543 | 110 | 60 | 22.64 |
| Q9QUI0 | ras homolog gene family, member A; similar to aplysia ras-related | 1333 | 21782 | 33 | 16 | 26.42 |
| | homolog A2; | | | | | |
| Q8BK67 | regulator of chromosome condensation 2; hypothetical protein | 299 | 55983 | 27 | 44 | 10.58 |
| | LOC100047340 | | | | | |
| P07356 | Annexin A2 | 2416 | 38676 | 95 | 31 | 44.54 |
| P15532 | Nucleoside diphosphate kinase A (NDK A) (NDP kinase A) | 1894 | 17208 | 35 | 16 | 26.97 |
| | (NM23A) | | | | | |
| P54116 | stomatin | 5278 | 31375 | 73 | 23 | 50.7 |
| P80317 | T complex protein 1 subunit zeta OS Mus musculus GN Cct6a PE | 316 | 58004 | 20 | 42 | 11.11 |
| | 1 SV 3 | | | | | |
| P80315 | T complex protein 1 subunit delta OS Mus musculus GN Cct4 PE | 186 | 58067 | 25 | 42 | 17.25 |
| | 1 SV 3 | | | | | |
| P11983 | t-complex protein 1 | 386 | 60449 | 48 | 46 | 22.48 |
| Q9WVA4 | Transgelin 2 OS Mus musculus GN Tagln2 PE 1 SV 4 | 190 | 22396 | 16 | 22 | 20.1 |
| P68373 | tubulin, alpha 1C; predicted gene 6682 | 4888 | 49910 | 112 | 34 | 32.96 |
| P68368 | tubulin, alpha 4A | 3251 | 49925 | 69 | 34 | 23.66 |
| Q9JJZ2 | tubulin, alpha 8 | 1697 | 50052 | 54 | 34 | 18.71 |
| Q7TMM9 | tubulin, beta 2A | 4491 | 49907 | 143 | 30 | 38.88 |
| Q9ERD7 | tubulin, beta 3; tubulin, beta 3, pseudogene 1 | 3145 | 50419 | 113 | 30 | 25.78 |
| Q9D6F9 | tubulin, beta 4 | 2370 | 49586 | 124 | 30 | 44.14 |
| P99024 | tubulin, beta 5 | 5109 | 49671 | 166 | 30 | 42.34 |
| Q9WV55 | vesicle-associated membrane protein | 159 | 27855 | 8 | 21 | 10.84 |
| Q922F4 | tubulin, beta 6 | 1925 | 50091 | 87 | 31 | 19.02 |
| Peripheral | | | | | | |
| Q9JKF1 | IQ motif containing GTPase activating protein 1 | 124 | 188743 | 61 | 118 | 6.22 |
| P24270 | Catalase OS Mus musculus GN Cat PE 1 SV 4 | 299 | 59795 | 16 | 48 | 18.98 |
| Q68FD5 | clathrin, heavy polypeptide (Hc) | 366 | 191557 | 94 | 133 | 14.15 |
| O55029 | coatomer protein complex, subunit beta 2 (beta prime) | 158 | 102449 | 30 | 69 | 7.07 |
| Q9QZE5 | coatomer protein complex, subunit gamma | 197 | 97513 | 35 | 68 | 13.62 |
| P05202 | glutamate oxaloacetate transaminase 2, mitochondrial | 1315 | 47412 | 55 | 45 | 27.21 |
| P08113 | heat shock protein 90, beta (Grp94), member 1 | 2700 | 92476 | 208 | 81 | 34.66 |
| P11438 | lysosomal-associated membrane protein 1 | 1502 | 43865 | 77 | 28 | 26.11 |
| P17047 | lysosomal-associated membrane protein 2 | 1444 | 45681 | 43 | 26 | 12.05 |
| P63101 | 14 3 3 protein zeta delta OS Mus musculus GN Ywhaz PE 1 SV 1 | 2795 | 27771 | 90 | 25 | 54.29 |
| P68254 | 14 3 3 protein theta OS Mus musculus GN Ywhaq PE 1 SV 1 | 882 | 27778 | 30 | 24 | 20 |
| P68510 | 14 3 3 protein eta OS Mus musculus GN Ywhah PE 1 SV 2 | 880 | 28212 | 28 | 27 | 24.8 |
| Q9CQV8 | 14 3 3 protein beta alpha OS Mus musculus GN Ywhab PE 1 SV | 1052 | 28086 | 42 | 22 | 39.43 |
| | 3 | | | | | |
| P63038 | heat shock protein 1 (chaperonin) | 2854 | 60956 | 154 | 53 | 43.8 |
| P17182 | enolase 1, alpha non-neuron | 221 | 47141 | 19 | 30 | 8.53 |
| Q01768 | Nucleoside diphosphate kinase B | 2044 | 17363 | 49 | 14 | 32.24 |
| P09103 | prolyl 4-hydroxylase, beta polypeptide | 2426 | 57059 | 127 | 48 | 55.6 |
| Q9WV80 | sorting nexin 1 | 142 | 58952 | 32 | 40 | 6.13 |
| P62983 | Ubiquitin 40S ribosomal protein S27a OS Mus musculus GN | 14724 | 17951 | 60 | 10 | 40.38 |
| | Rps27a PE 1 SV 2 | | | | | |
| P20029 | 78 kDa glucose regulated protein OS Mus musculus Grp78 | 7714 | 72422 | 243 | 52 | 39.85 |
| P24668 | Cation dependent mannose 6 phosphate receptor | 3533 | 31173 | 83 | 25 | 28.06 |
| P58021 | Transmembrane 9 superfamily member 2 OS Mus musculus GN | 283 | 75330 | 18 | 28 | 4.08 |
| | Tm9sf2 PE 2 SV 1 | | | | | |
| P27773 | Protein disulfide isomerase A3 OS Mus musculus GN Pdia3 PE 1 | 3322 | 56678 | 162 | 55 | 37.23 |
| | SV 2 | | | | | |
| Q922R8 | Protein disulfide isomerase A6 OS Mus musculus GN Pdia6 PE 1 | 2904 | 48100 | 80 | 34 | 27.95 |
| | SV 3 | | | | | |
| Q9CWK8 | Sorting nexin 2 OS Mus musculus GN Snx2 PE 1 SV 2 | 267 | 58471 | 37 | 37 | 9.63 |
| P51863 | V type proton ATPase subunit d 1 OS Mus musculus GN | 401 | 40301 | 33 | 22 | 11.4 |
| | Atp6v0d1 PE 1 SV 2 | | | | | |
| P16627 | Heat shock 70 kDa protein 1 like OS Mus musculus GN Hspa11 | 1300 | 70637 | 64 | 51 | 29.49 |
| | PE 2 SV 4 | | | | | |
| P17156 | Heat shock related 70 kDa protein 2 OS Mus musculus GN Hspa2 | 3988 | 69642 | 139 | 50 | 28.44 |
| | PE 1 SV 2 | | | | | |
| P07901 | Heat shock protein HSP 90 alpha OS Mus musculus GN | 1385 | 84788 | 37 | 91 | 14.05 |
| | Hsp90aa1 PE 1 SV 4 | | | | | |
| P35700 | Peroxiredoxin 1 OS Mus musculus GN Prdx1 PE 1 SV 1 | 2841 | 22177 | 64 | 18 | 37.69 |
| Q03265 | ATP synthase subunit alpha mitochondrial OS Mus musculus GN | 5468 | 59753 | 185 | 46 | 33.45 |
| | Atp5a1 PE 1 SV 1 | | | | | |
| P17047 | Lysosome associated membrane glycoprotein 2 OS Mus musculus | 1444 | 45681 | 43 | 26 | 12.05 |
| | GN Lamp2 PE 2 SV 2 | | | | | |
| Q8VEK3 | Heterogeneous nuclear ribonucleoprotein U OS Mus musculus | 1113 | 87918 | 73 | 53 | 23.38 |
| | GN Hnrnpu PE 1 SV 1 | | | | | |
| P11499 | Heat shock protein HSP 90 beta OS Mus musculus GN Hsp90ab1 | 1773 | 83281 | 78 | 67 | 25 |
| | PE 1 SV 3 | | | | | |
| P17879 | Heat shock 70 kDa protein 1B OS Mus musculus GN Hspa1b PE | 1444 | 70176 | 58 | 48 | 23.05 |
| | 1 SV 3 | | | | | |
| Q62167 | ATP dependent RNA helicase DDX3X OS Mus musculus GN | 708 | 73102 | 42 | 65 | 16.16 |
| | Ddx3x PE 1 SV 3 | | | | | |
| P08003 | Protein disulfide isomerase A4 OS Mus musculus GN Pdia4 PE 1 | 2374 | 71983 | 141 | 66 | 31.35 |
| | SV 3 | | | | | |
| O35129 | Prohibitin 2 OS Mus musculus GN Phb2 PE 1 SV 1 | 2516 | 33296 | 66 | 29 | 30.1 |
| P38647 | Stress 70 protein mitochondrial OS Mus musculus GN Hspa9 PE | 768 | 73461 | 71 | 68 | 33.28 |
| | 1 SV 3 | | | | | |
| O55143 | Sarcoplasmic endoplasmic reticulum calcium ATPase 2 OS | 662 | 114858 | 121 | 73 | 28.45 |
| | Mus musculus GN Atp2a2 PE 1 SV 2 | | | | | |
| P68040 | Guanine nucleotide binding protein subunit beta 2 like 1 OS | 966 | 35077 | 72 | 27 | 25.87 |
| | Mus musculus GN Gnb211 PE 1 SV 3 | | | | | |
| P08752 | Guanine nucleotide binding protein G i subunit alpha 2 OS | 1686 | 40489 | 50 | 29 | 29.58 |
| | Mus musculus GN Gnai2 PE 1 SV 5 | | | | | |
| P67778 | Prohibitin OS Mus musculus GN Phb PE 1 SV 1 | 5730 | 29820 | 106 | 21 | 44.12 |
| Membrane | | | | | | |
| vesicles - | | | | | | |
| secreted | | | | | | |
| P14211 | calreticulin | 9639 | 47995 | 251 | 50 | 54.33 |
| P10605 | cathepsin B | 303 | 37280 | 12 | 26 | 4.13 |
| P48036 | Annexin A5 OS Mus musculus GN Anxa5 PE 1 SV 1 | 364 | 35753 | 30 | 30 | 18.81 |
| P07356 | annexin A2 | 2416 | 38676 | 95 | 31 | 44.54 |
| O70456 | 14 3 3 protein sigma OS Mus musculus GN Sfn PE 1 SV 2 | 819 | 27706 | 34 | 24 | 26.21 |
| P18242 | Cathepsin D OS Mus musculus GN Ctsd PE 1 SV 1 | 4638 | 44954 | 107 | 24 | 20 |
| P29391 | Ferritin light chain 1 OS Mus musculus GN Ftl1 PE 1 SV 2 | 1037 | 20802 | 34 | 17 | 41.53 |
| Q9D1D4 | Transmembrane emp24 domain containing protein 10 OS Mus musculus | 3862 | 24911 | 45 | 21 | 19.63 |
| | GN Tmed10 PE 2 SV 1 | | | | | |
| Q99KF1 | Transmembrane emp24 domain containing protein 9 OS Mus musculus | 1338 | 27127 | 34 | 20 | 31.49 |
| | GN Tmed9 PE 2 SV 2 | | | | | |
| P07724 | Serum albumin OS Mus musculus GN Alb PE 1 SV 3 | 133 | 68693 | 16 | 54 | 7.57 |
| Q9WUU7 | Cathepsin Z OS Mus musculus GN Ctsz PE 2 SV 1 | 681 | 33996 | 43 | 20 | 18.3 |
| O70503 | Estradiol 17 beta dehydrogenase 12 OS Mus musculus GN | 1194 | 34742 | 54 | 21 | 29.17 |
| | Hsd17b12 PE 2 SV 1 | | | | | |
| P17742 | Peptidyl prolyl cis trans isomerase A OS Mus musculus GN Ppia | 219 | 17971 | 5 | 12 | 6.71 |
| | PE 1 SV 2 | | | | | |
| Q07797 | Galectin 3 binding protein OS Mus musculus GN Lgals3bp PE 1 | 362 | 64491 | 43 | 31 | 18.89 |
| | SV 1 | | | | | |
| P62960 | Nuclease sensitive element binding protein 1 OS Mus musculus | 475 | 35730 | 20 | 26 | 15.84 |
| | GN Ybx1 PE 1 SV 3 | | | | | |
| Q9DBG6 | Dolichyl diphosphooligosaccharide protein glycosyltransferase | 709 | 69063 | 51 | 38 | 15.06 |
| | subunit 2 OS | | | | | |
| Q8BPX9 | Solute carrier family 15 member 3 OS Mus musculus GN Slc15a3 | 617 | 64051 | 17 | 31 | 3.46 |
| | PE 2 SV 1 | | | | | |
| Q8VEH3 | ADP ribosylation factor like protein 8A OS Mus musculus GN | 275 | 21390 | 33 | 22 | 17.74 |
| | Arl8a PE 2 SV 1 | | | | | |
| Q9CYN2 | Signal peptidase complex subunit 2 OS Mus musculus GN Spcs2 | 146 | 24978 | 11 | 20 | 7.96 |
| | PE 2 SV 1 | | | | | |
| P61982 | 14 3 3 protein gamma OS Mus musculus GN Ywhag PE 1 SV 2 | 1094 | 28303 | 28 | 25 | 12.96 |
| Q9QUJ7 | Long chain fatty acid CoA ligase 4 OS Mus musculus GN Acsl4 | 169 | 79077 | 32 | 51 | 11.39 |
| | PE 2 SV 2 | | | | | |
| P62259 | 14 3 3 protein epsilon OS Mus musculus GN Ywhae PE 1 SV 1 | 1547 | 29174 | 53 | 29 | 40.78 |
| P12265 | Beta glucuronidase OS Mus musculus GN Gusb PE 2 SV 2 | 144 | 74195 | 18 | 49 | 4.78 |
| O08547 | Vesicle trafficking protein SEC22b OS Mus musculus GN Sec22b | 570 | 24741 | 18 | 17 | 21.4 |
| | PE 1 SV 3 | | | | | |
| Mitochondrion | | | | | | |
| O08756 | 3 hydroxyacyl CoA dehydrogenase type 2 OS Mus musculus GN | 375 | 27419 | 21 | 20 | 15.33 |
| | Hsd17b10 PE 1 SV 4 | | | | | |
| Q99KI0 | Aconitate hydratase mitochondrial OS Mus musculus GN Aco2 | 606 | 85464 | 86 | 55 | 20.38 |
| | PE 1 SV 1 | | | | | |
| P48962 | ADP ATP translocase 1 OS Mus musculus GN Slc25a4 PE 1 SV | 613 | 32904 | 33 | 31 | 19.8 |
| | 4 | | | | | |
| P51881 | ADP ATP translocase 2 OS Mus musculus GN Slc25a5 PE 1 SV | 1951 | 32931 | 53 | 31 | 27.52 |
| | 3 | | | | | |
| P47738 | Aldehyde dehydrogenase mitochondrial OS Mus musculus GN | 2682 | 56538 | 83 | 40 | 23.12 |
| | Aldh2 PE 1 SV 1 | | | | | |
| P56480 | ATP synthase subunit beta mitochondrial OS Mus musculus GN | 6085 | 56301 | 202 | 37 | 47.64 |
| | Atp5b PE 1 SV 2 | | | | | |
| Q9DCX2 | ATP synthase subunit d mitochondrial OS Mus musculus GN | 636 | 18749 | 22 | 12 | 54.66 |
| | Atp5h PE 1 SV 3 | | | | | |
| Q9DB20 | ATP synthase, H+ transporting, mitochondrial F1 complex, O | 758 | 23364 | 20 | 18 | 26.76 |
| | subunit | | | | | |
| Q9QXX4 | Calcium binding mitochondrial carrier protein Aralar2 OS | 178 | 74467 | 30 | 51 | 15.83 |
| | Mus musculus GN Slc25a13 PE 1 SV 1 | | | | | |
| P08074 | Carbonyl reductase NADPH 2 OS Mus musculus GN Cbr2 PE 1 | 284 | 25958 | 27 | 21 | 18.03 |
| | SV 1 | | | | | |
| Q9CZU6 | Citrate synthase mitochondrial OS Mus musculus GN Cs PE 1 SV | 1015 | 51737 | 67 | 33 | 22.41 |
| | 1 | | | | | |
| Q9DCN2 | cytochrome b5 reductase 3 | 320 | 34128 | 20 | 24 | 14.95 |
| Q9CZ13 | Cytochrome b c1 complex subunit 1 mitochondrial OS Mus musculus | 356 | 52852 | 33 | 38 | 18.54 |
| | GN Uqcrc1 PE 1 SV 2 | | | | | |
| Q9DB77 | Cytochrome b c1 complex subunit 2 mitochondrial OS Mus musculus | 199 | 48235 | 25 | 26 | 17.66 |
| | GN Uqcrc2 PE 1 SV 1 | | | | | |
| P00405 | Cytochrome c oxidase subunit 2 OS Mus musculus GN Mtco2 PE | 2978 | 25977 | 72 | 11 | 20.26 |
| | 1 SV 1 | | | | | |
| O08749 | Dihydrolipoyl dehydrogenase mitochondrial OS Mus musculus | 159 | 54272 | 13 | 30 | 5.3 |
| | GN Dld PE 1 SV 2 | | | | | |
| Q9D2G2 | Dihydrolipoyllysine residue succinyltransferase component of 2 | 987 | 48995 | 32 | 34 | 11.01 |
| | oxoglutarate dehydrogenase complex | | | | | |
| Q99LC5 | Electron transfer flavoprotein subunit alpha mitochondrial OS | 417 | 35010 | 22 | 24 | 29.13 |
| | Mus musculus GN Etfa PE 1 SV 2 | | | | | |
| Q9DCW4 | Electron transfer flavoprotein subunit beta OS Mus musculus GN | 583 | 27623 | 33 | 17 | 32.55 |
| | Etfb PE 1 SV 3 | | | | | |
| Q8BFR5 | Elongation factor Tu mitochondrial OS Mus musculus GN Tufm | 871 | 49508 | 62 | 40 | 30.53 |
| | PE 1 SV 1 | | | | | |
| P97807 | Fumarate hydratase mitochondrial OS Mus musculus GN Fh PE 1 | 288 | 54357 | 15 | 34 | 6.9 |
| | SV 3 | | | | | |
| P26443 | Glutamate dehydrogenase 1 mitochondrial OS Mus musculus GN | 1096 | 61337 | 115 | 44 | 34.77 |
| | Glud1 PE 1 SV 1 | | | | | |
| Q9D964 | Glycine amidinotransferase mitochondrial OS Mus musculus GN | 312 | 48297 | 18 | 36 | 4.02 |
| | Gatm PE 1 SV 1 | | | | | |
| Q9D6R2 | Isocitrate dehydrogenase NAD subunit mitochondrial OS | 537 | 39639 | 27 | 31 | 11.48 |
| | Mus musculus GN Idh3a PE 1 SV 1 | | | | | |
| P54071 | Isocitrate dehydrogenase NADP mitochondrial OS Mus musculus | 613 | 50906 | 46 | 37 | 17.7 |
| | GN Idh2 PE 1 SV 3 | | | | | |
| Q8CGK3 | Lon protease homolog mitochondrial OS Mus musculus GN | 158 | 105843 | 42 | 90 | 8.96 |
| | Lonp1 PE 1 SV 2 | | | | | |
| P51174 | Long chain specific acyl CoA dehydrogenase mitochondrial OS | 683 | 47908 | 26 | 31 | 19.53 |
| | Mus musculus GN Acadl PE 2 SV 2 | | | | | |
| P08249 | Malate dehydrogenase mitochondrial OS Mus musculus GN | 5184 | 35612 | 114 | 30 | 45.56 |
| | Mdh2 PE 1 SV 3 | | | | | |
| P29758 | Ornithine aminotransferase mitochondrial OS Mus musculus GN | 759 | 48355 | 54 | 32 | 41.91 |
| | Oat PE 1 SV 1 | | | | | |
| Q8BH04 | Phosphoenolpyruvate carboxykinase GTP mitochondrial OS Mus musculus | 591 | 70528 | 55 | 53 | 24.06 |
| | GN Pck2 PE 2 SV 1 | | | | | |
| Q922W5 | Pyrroline 5 carboxylate reductase 1 mitochondrial OS Mus musculus | 152 | 32374 | 10 | 24 | 15.21 |
| | GN Pycr1 PE 1 SV 1 | | | | | |
| Q8K2B3 | Succinate dehydrogenase ubiquinone flavoprotein subunit | 321 | 72586 | 43 | 47 | 20.63 |
| | mitochondrial OS | | | | | |
| Q9D0K2 | Succinyl CoA 3 ketoacid coenzyme A transferase 1 mitochondrial | 225 | 55989 | 36 | 39 | 12.69 |
| | OS | | | | | |
| Q9R112 | Sulfide quinone oxidoreductase mitochondrial OS Mus musculus | 542 | 50283 | 36 | 45 | 22.67 |
| | GN Sqrdl PE 2 SV 3 | | | | | |
| Q8BMS1 | Trifunctional enzyme subunit alpha mitochondrial OS | 179 | 82670 | 38 | 50 | 14.94 |
| | Mus musculus GN Hadha PE 1 SV 1 | | | | | |
| Q60932 | Voltage dependent anion selective channel protein 1 OS | 3309 | 32352 | 81 | 21 | 45.95 |
| | Mus musculus GN Vdac1 PE 1 SV 3 | | | | | |
| Q60930 | Voltage dependent anion selective channel protein 2 OS | 6899 | 31733 | 108 | 20 | 35.59 |
| | Mus musculus GN Vdac2 PE 1 SV 2 | | | | | |
| Q60931 | Voltage dependent anion selective channel protein 3 OS | 2691 | 30753 | 52 | 19 | 26.5 |
| | Mus musculus GN Vdac3 PE 1 SV 1 | | | | | |
| Ribosome | | | | | | |
| P14131 | 40S ribosomal protein S16 OS Mus musculus GN Rps16 PE 2 SV 4 | 3821 | 35810 | 88 | 19 | 37.24 |
| P25444 | 40S ribosomal protein S2 OS Mus musculus GN Rps2 PE 1 SV 3 | 285 | 31231 | 24 | 27 | 21.16 |
| P62908 | 40S ribosomal protein S3 OS Mus musculus GN Rps3 PE 1 SV 1 | 871 | 26674 | 51 | 26 | 30.04 |
| P62702 | 40S ribosomal protein S4 X isoform OS Mus musculus GN Rps4x | 1314 | 29598 | 37 | 20 | 16.35 |
| | PE 2 SV 2 | | | | | |
| P62754 | 40S ribosomal protein S6 OS Mus musculus GN Rps6 PE 1 SV 1 | 426 | 28681 | 26 | 20 | 19.28 |
| P62242 | 40S ribosomal protein S8 OS Mus musculus GN Rps8 PE 1 SV 2 | 1180 | 24205 | 50 | 16 | 46.63 |
| P14869 | 60S acidic ribosomal protein P0 OS Mus musculus GN Rplp0 PE | 2938 | 34216 | 48 | 20 | 27.76 |
| | 1 SV 3 | | | | | |
| P47955 | 60S acidic ribosomal protein P1 OS Mus musculus GN Rplp1 PE | 7094 | 11475 | 27 | 7 | 51.75 |
| | 1 SV 1 | | | | | |
| Q6ZWV3 | 60S ribosomal protein L10 OS Mus musculus GN Rpl10 PE 2 SV 3 | 653 | 24604 | 12 | 14 | 21.96 |
| Q9CXW4 | 60S ribosomal protein L11 OS Mus musculus GN Rpl11 PE 1 SV 4 | 1741 | 20252 | 19 | 16 | 25.84 |
| P35979 | 60S ribosomal protein L12 OS Mus musculus GN Rpl12 PE 1 SV 2 | 4005 | 17805 | 46 | 15 | 35.76 |
| P47963 | 60S ribosomal protein L13 OS Mus musculus GN Rpl13 PE 2 SV 3 | 613 | 24306 | 14 | 18 | 23.22 |
| Q9CR57 | 60S ribosomal protein L14 OS Mus musculus GN Rpl14 PE 2 SV 3 | 2891 | 23564 | 59 | 6 | 30.88 |
| Q9CZM2 | 60S ribosomal protein L15 OS Mus musculus GN Rpl15 PE 2 SV 4 | 760 | 24146 | 13 | 15 | 10.29 |
| P35980 | 60S ribosomal protein L18 OS Mus musculus GN Rpl18 PE 2 SV 3 | 3024 | 21645 | 60 | 10 | 34.04 |
| P62717 | 60S ribosomal protein L18a OS Mus musculus GN Rpl18a PE 1 SV 1 | 2092 | 20732 | 35 | 19 | 15.91 |
| O09167 | 60S ribosomal protein L21 OS Mus musculus GN Rpl21 PE 2 SV 3 | 977 | 18562 | 53 | 13 | 45.63 |
| P67984 | 60S ribosomal protein L22 OS Mus musculus GN Rpl22 PE 2 SV 2 | 1724 | 14759 | 19 | 7 | 30.47 |
| P62830 | 60S ribosomal protein L23 OS Mus musculus GN Rpl23 PE 1 SV 1 | 3245 | 14865 | 21 | 13 | 20 |
| Q8BP67 | 60S ribosomal protein L24 OS Mus musculus GN Rpl24 PE 2 SV 2 | 989 | 17779 | 31 | 16 | 19.11 |
| P61358 | 60S ribosomal protein L27 OS Mus musculus GN Rpl27 PE 2 SV 2 | 2826 | 15798 | 46 | 9 | 36.76 |
| P27659 | 60S ribosomal protein L3 OS Mus musculus GN Rpl3 PE 2 SV 3 | 570 | 46110 | 52 | 35 | 26.55 |
| P62889 | 60S ribosomal protein L30 OS Mus musculus GN Rpl30 PE 2 SV 2 | 7155 | 12784 | 40 | 7 | 48.7 |
| Q9D1R9 | 60S ribosomal protein L34 OS Mus musculus GN Rpl34 PE 3 SV 2 | 1091 | 13293 | 29 | 7 | 12.82 |
| P47964 | 60S ribosomal protein L36 OS Mus musculus GN Rpl36 PE 2 SV 2 | 680 | 12216 | 15 | 9 | 36.19 |
| Q9D8E6 | 60S ribosomal protein L4 OS Mus musculus GN Rpl4 PE 1 SV 3 | 1791 | 47154 | 75 | 31 | 22.2 |
| P47911 | 60S ribosomal protein L6 OS Mus musculus GN Rpl6 PE 1 SV 3 | 857 | 33510 | 44 | 24 | 31.42 |
| P14148 | 60S ribosomal protein L7 OS Mus musculus GN Rpl7 PE 2 SV 2 | 3497 | 31420 | 62 | 21 | 23.7 |
| P12970 | 60S ribosomal protein L7a OS Mus musculus GN Rpl7a PE 2 SV 2 | 1406 | 29977 | 34 | 19 | 22.93 |
| P62918 | 60S ribosomal protein L8 OS Mus musculus GN Rpl8 PE 2 SV 2 | 1198 | 28025 | 36 | 21 | 29.96 |
| Nucleus | | | | | | |
| P25206 | DNA replication licensing factor MCM3 OS Mus musculus GN Mcm3 | 304 | 91547 | 46 | 75 | 15.27 |
| | PE 1 SV 2 | | | | | |
| Q99020 | Heterogeneous nuclear ribonucleoprotein A B OS Mus musculus | 3761 | 30831 | 57 | 22 | 20.7 |
| | GN Hnrnpab PE 1 SV 1 | | | | | |
| P49312 | Heterogeneous nuclear ribonucleoprotein A1 OS Mus musculus | 1150 | 34196 | 34 | 29 | 11.25 |
| | GN Hnrnpa1 PE 1 SV 2 | | | | | |
| Q8BG05 | Heterogeneous nuclear ribonucleoprotein A3 OS Mus musculus | 1741 | 39652 | 75 | 32 | 22.16 |
| | GN Hnrnpa3 PE 1 SV 1 | | | | | |
| Q60668 | Heterogeneous nuclear ribonucleoprotein DO OS Mus musculus | 894 | 38354 | 34 | 24 | 18.31 |
| | GN Hnrnpd PE 1 SV 2 | | | | | |
| Q9Z2X1 | Heterogeneous nuclear ribonucleoprotein F OS Mus musculus | 3684 | 45730 | 108 | 26 | 27.47 |
| | GN Hnrnpf PE 1 SV 3 | | | | | |
| O35737 | Heterogeneous nuclear ribonucleoprotein H OS Mus musculus | 1683 | 49200 | 62 | 27 | 23.83 |
| | GN Hnrnph1 PE 1 SV 3 | | | | | |
| P70333 | Heterogeneous nuclear ribonucleoprotein H2 OS Mus musculus | 787 | 49280 | 52 | 27 | 17.37 |
| | GN Hnrnph2 PE 1 SV 1 | | | | | |
| P61979 | Heterogeneous nuclear ribonucleoprotein K OS Mus musculus | 2589 | 50976 | 72 | 38 | 29.81 |
| | GN Hnrnpk PE 1 SV 1 | | | | | |
| O88569 | Heterogeneous nuclear ribonucleoproteins A2 B1 OS Mus musculus | 1717 | 37403 | 46 | 29 | 17.28 |
| | GN Hnrnpa2b1 PE 1 SV 2 | | | | | |
| Q9Z204 | Heterogeneous nuclear ribonucleoproteins C1 C2 OS Mus musculus | 196 | 34385 | 24 | 35 | 7.99 |
| | GN Hnrnpc PE 1 SV 1 | | | | | |
| P10922 | Histone H1 0 OS Mus musculus GN H1f0 PE 2 SV 4 | 521 | 20861 | 24 | 11 | 18.56 |
| P43275 | Histone H1 1 OS Mus musculus GN Hist1h1a PE 1 SV 2 | 1011 | 21785 | 17 | 17 | 9.86 |
| P43274 | Histone H1 4 OS Mus musculus GN Hist1h1e PE 1 SV 2 | 1377 | 21977 | 54 | 19 | 16.89 |
| Q8CGP6 | Histone H2A type 1 H OS Mus musculus GN Hist1h2ah PE 1 SV 3 | 31701 | 13950 | 195 | 7 | 31.25 |
| Q64522 | Histone H2A type 2 B OS Mus musculus GN Hist2h2ab PE 1 SV 3 | 32018 | 14013 | 185 | 8 | 30.77 |
| Q64523 | Histone H2A type 2 C OS Mus musculus GN Hist2h2ac PE 1 SV 3 | 31701 | 13988 | 198 | 7 | 31.01 |
| Q3THW5 | Histone H2A V OS Mus musculus GN H2afv PE 1 SV 3 | 1779 | 13509 | 52 | 7 | 14.84 |
| Q64475 | Histone H2B type 1 B OS Mus musculus GN Hist1h2bb PE 1 SV 3 | 20349 | 13952 | 139 | 11 | 38.1 |
| P62806 | Histone H4 OS Mus musculus GN Hist1h4a PE 1 SV 2 | 31977 | 11367 | 263 | 11 | 66.99 |
| Q3U9G9 | Lamin B receptor OS Mus musculus GN Lbr PE 1 SV 2 | 545 | 71440 | 31 | 45 | 10.7 |
| P09405 | Nucleolin OS Mus musculus GN Ncl PE 1 SV 2 | 2574 | 76723 | 147 | 59 | 20.79 |
| Q61937 | Nucleophosmin OS Mus musculus GN Npm1 PE 1 SV 1 | 5722 | 32560 | 83 | 22 | 14.04 |
| P17225 | Polypyrimidine tract binding protein 1 OS Mus musculus GN | 286 | 56478 | 23 | 36 | 9.49 |
| | Ptbp1 PE 1 SV 2 | | | | | |
| Q501J6 | Probable ATP dependent RNA helicase DDX17 OS Mus musculus GN | 1279 | 72400 | 75 | 60 | 26.15 |
| | Ddx17 PE 2 SV 1 | | | | | |
| Q61656 | Probable ATP dependent RNA helicase DDX5 OS Mus musculus GN | 1782 | 69290 | 120 | 59 | 31.76 |
| | Ddx5 PE 1 SV 2 | | | | | |
| P56959 | RNA binding protein FUS OS Mus musculus GN Fus PE 2 SV 1 | 306 | 52673 | 24 | 19 | 9.46 |
| Q6PDM2 | Serine arginine rich splicing factor 1 OS Mus musculus GN Srsf1 | 1133 | 27745 | 38 | 29 | 24.6 |
| | PE 1 SV 3 | | | | | |
| P84104 | Serine arginine rich splicing factor 3 OS Mus musculus GN Srsf3 | 1140 | 19330 | 13 | 14 | 18.29 |
| | PE 1 SV 1 | | | | | |
| O35326 | Serine arginine rich splicing factor 5 OS Mus musculus GN Srsf5 | 429 | 30891 | 18 | 18 | 3.35 |
| | PE 1 SV 2 | | | | | |
| P62320 | Small nuclear ribonucleoprotein Sm D3 OS Mus musculus GN | 2813 | 13916 | 32 | 9 | 22.22 |
| | Snrpd3 PE 1 SV 1 | | | | | |
| Q9Z1N5 | Spliceosome RNA helicase Ddx39b OS Mus musculus GN Ddx39b | 610 | 49036 | 42 | 37 | 27.1 |
| | PE 1 SV 1 | | | | | |
| Q921F2 | TAR DNA binding protein 43 OS Mus musculus GN Tardbp PE | 393 | 44548 | 25 | 18 | 18.6 |
| | 1 SV 1 | | | | | |
| P40142 | Transketolase OS Mus musculus GN Tkt PE 1 SV 1 | 230 | 67631 | 26 | 44 | 14.45 |
| Cytoplasm | | | | | | |
| P63017 | Heat shock cognate 71 kDa protein OS Mus musculus GN Hspa8 | 5757 | 70871 | 247 | 50 | 45.2 |
| | PE 1 SV 1 | | | | | |
| P10126 | Elongation factor 1 alpha 1 OS Mus musculus GN Eef1a1 PE 1 | 2952 | 50114 | 130 | 32 | 30.74 |
| | SV 3 | | | | | |
| Q91VC3 | Eukaryotic initiation factor 4A III OS Mus musculus GN Eif4a3 | 518 | 46840 | 39 | 48 | 17.03 |
| | PE 2 SV 3 | | | | | |
| P52480 | Pyruvate kinase isozymes M1 M2 OS Mus musculus GN Pkm PE 1 | 4539 | 57845 | 187 | 45 | 41.62 |
| | SV 4 | | | | | |
| P58252 | Elongation factor 2 OS Mus musculus GN Eef2 PE 1 SV 2 | 2082 | 95314 | 200 | 69 | 35.78 |
| P00342 | L lactate dehydrogenase C chain OS Mus musculus GN Ldhc PE | 446 | 35912 | 23 | 25 | 12.35 |
| | 1 SV 2 | | | | | |
| P05064 | Fructose bisphosphate aldolase A OS Mus musculus GN Aldoa PE | 805 | 39356 | 46 | 31 | 23.9 |
| | 1 SV 2 | | | | | |
| P50431 | Serine hydroxymethyltransferase cytosolic OS Mus musculus GN | 366 | 52601 | 26 | 38 | 2.93 |
| | Shmt1 PE 1 SV 3 | | | | | |
| P16125 | L lactate dehydrogenase B chain OS Mus musculus GN Ldhb PE 1 | 404 | 36572 | 11 | 25 | 13.17 |
| | SV 2 | | | | | |
| Q9DCD0 | 6 phosphogluconate dehydrogenase decarboxylating OS Mus musculus | 248 | 53247 | 30 | 38 | 19.25 |
| | GN Pgd PE 2 SV 3 | | | | | |
| P06151 | L lactate dehydrogenase A chain OS Mus musculus GN Ldha PE 1 | 1125 | 36499 | 43 | 28 | 28.31 |
| | SV 3 | | | | | |
| Q78PY7 | Staphylococcal nuclease domain containing protein 1 OS Mus musculus | 285 | 102088 | 35 | 83 | 14.62 |
| | GN Snd1 PE 1 SV 1 | | | | | |
| P70670 | Nascent polypeptide associated complex subunit alpha muscle | 508 | 220500 | 35 | 175 | 6.31 |
| | specific form OS | | | | | |
| P62315 | Small nuclear ribonucleoprotein Sm D1 OS Mus musculus GN | 576 | 13282 | 17 | 6 | 27.73 |
| | Snrpd1 PE 1 SV 1 | | | | | |
| Q7TMK9 | Heterogeneous nuclear ribonucleoprotein Q OS Mus musculus GN | 601 | 69633 | 56 | 50 | 15.89 |
| | Syncrip PE 1 SV 2 | | | | | |
| P29341 | Polyadenylate binding protein 1 OS Mus musculus GN Pabpc1 PE | 766 | 70671 | 84 | 45 | 28.14 |
| | 1 SV 2 | | | | | |
| P47962 | 60S ribosomal protein L5 OS Mus musculus GN Rpl5 PE 1 SV 3 | 862 | 34401 | 36 | 23 | 16.5 |
| Q8R081 | Heterogeneous nuclear ribonucleoprotein L OS Mus musculus GN | 115 | 63964 | 25 | 34 | 13.14 |
| | Hnrnpl PE 1 SV 2 | | | | | |
| P62307 | Small nuclear ribonucleoprotein F OS Mus musculus GN Snrpf PE | 1851 | 9725 | 10 | 6 | 15.12 |
| | 2 SV 1 | | | | | |
| P70372 | ELAV like protein 1 OS Mus musculus GN Elavl1 PE 1 SV 2 | 679 | 36169 | 32 | 23 | 27.3 |
| O70133 | ATP dependent RNA helicase A OS Mus musculus GN Dhx9 PE | 138 | 149475 | 35 | 100 | 4.93 |
| | 1 SV 2 | | | | | |
| P16858 | Glyceraldehyde 3 phosphate dehydrogenase OS Mus musculus GN | 3821 | 35810 | 88 | 19 | 37.24 |
| | Gapdh PE 1 SV 2 | | | | | |
| ER | | | | | | |
| Q9JKR6 | Hypoxia up regulated protein 1 OS Mus musculus GN Hyou1 PE | 764 | 111181 | 101 | 90 | 22.72 |
| | 1 SV 1 | | | | | |
| Q6P5E4 | UDP glucose glycoprotein glucosyltransferase 1 OS Mus musculus | 205 | 176434 | 69 | 113 | 10.7 |
| | GN Uggt1 PE 1 SV 4 | | | | | |
| Q99KV1 | DnaJ homolog subfamily B member 11 OS Mus musculus GN | 219 | 40555 | 20 | 27 | 4.47 |
| | Dnajb11 PE 1 SV 1 | | | | | |
| P24369 | Peptidyl prolyl cis trans isomerase B OS Mus musculus GN Ppib | 1781 | 23714 | 43 | 21 | 32.87 |
| | PE 2 SV 2 | | | | | |
| Q64518 | Sarcoplasmic endoplasmic reticulum calcium ATPase 3 OS | 209 | 113638 | 33 | 72 | 10.6 |
| | Mus musculus GN Atp2a3 PE 2 SV 3 | | | | | |
| P35564 | Calnexin OS Mus musculus GN Canx PE 1 SV 1 | 1760 | 67278 | 91 | 44 | 25.04 |
| Q91W90 | Thioredoxin domain containing protein 5 OS Mus musculus GN | 121 | 46416 | 16 | 32 | 11.27 |
| | Txndc5 PE 1 SV 2 | | | | | |
| Q01853 | Transitional endoplasmic reticulum ATPase OS Mus musculus GN | 631 | 89322 | 80 | 69 | 25.81 |
| | Vcp PE 1 SV 4 | | | | | |
| Q62186 | Translocon associated protein subunit delta OS Mus musculus GN | 1547 | 18937 | 26 | 9 | 25 |
| | Ssr4 PE 2 SV 1 | | | | | |
| O54734 | Dolichyl diphosphooligosaccharide protein glycosyltransferase 48 | 620 | 49028 | 22 | 25 | 11.11 |
| | kDa subunit OS Mus musculus GN Ddo | | | | | |
| Q8BHN3 | Neutral alpha glucosidase AB OS Mus musculus GN Ganab PE 1 | 236 | 106911 | 64 | 69 | 12.92 |
| | SV 1 | | | | | |
| Q91YQ5 | Dolichyl diphosphooligosaccharide protein glycosyltransferase | 840 | 68528 | 63 | 49 | 27.14 |
| | subunit 1 OS Mus musculus GN Rpn1 PE | | | | | |
| Other | | | | | | |
| P60843 | Eukaryotic initiation factor 4A I OS Mus musculus GN Eif4a1 | 1599 | 46154 | 55 | 46 | 25.37 |
| | PE 2 SV 1 | | | | | |
| Q8VCH0 | 3 ketoacyl CoA thiolase B peroxisomal OS Mus musculus GN | 223 | 43996 | 18 | 26 | 12.97 |
| | Acaa1b PE 2 SV 1 | | | | | |

TABLE 3
PHYSICAL PROPERTIES OF LEUKOSOMES

| | | |
|---|---|---|
| Density | 1.48 | g/cm³ |
| Mass of Lipids | 0.02 | g |
| Radius of LK | 6.1 × 10⁻⁶ | cm |
| Mass per LK | 1.41 × 10⁻¹⁵ | g particles |
| Number of Particles | 1.42 × 10¹³ | particles |
| Surface Area | 0.0467 | μm² |
| Total Surface Area | 6.65 × 10¹¹ | μm² |

TABLE 4
THEORETICAL CALCULATIONS OF LEUKOSOME SURFACE MARKERS

| | LFA-1 | MAC-1 | PSGL-1 | CD18 | CD45 | CD47 | IgG |
|---|---|---|---|---|---|---|---|
| # of copies | 1.37 × 10¹⁴ | 9.93 × 10¹³ | 5.62 × 10¹³ | 9.60 × 10¹³ | 7.24 × 10¹³ | 1.24 × 10¹³ | 7.78 × 10¹³ |
| #of copies/particle | ≈10 | ≈7 | ≈4 | ≈7 | ≈5 | ≈9 | ≈0.055 |
| #of copies/surface | ≈206 | ≈149 | ≈85 | ≈144 | ≈109 | ≈187 | ≈1 |
| area(μm²) | | | | | | | |

Leukosomes displayed preferential targeting of inflamed endothelia, both in vitro and in vivo. For these studies DXM, an anti-inflammatory glucocorticoid (Franchimont et al., 2002), was selected to demonstrate the therapeutic potential of leukosomes. DXM encapsulation did not affect the surface identity of leukosomes, indicating that the carrier's surface properties were preserved after drug loading. A flow chamber assay was used to test the ability of liposomes and leukosomes to adhere to a reconstructed monolayer of activated endothelial cells (HUVEC), under physiologically relevant shear stresses. Compared to conventional liposomes, leukosomes preferentially recognized the inflamed endothelium. To demonstrate successful treatment, PCR analysis was performed and it was observed that DXM-loaded leukosomes reduced the expression of pro-inflammatory markers (CCL2 and IL6) and endothelial adhesion molecules (ICAM-1 and VCAM-1) as well as increased levels of the anti-inflammatory gene MRC-1.

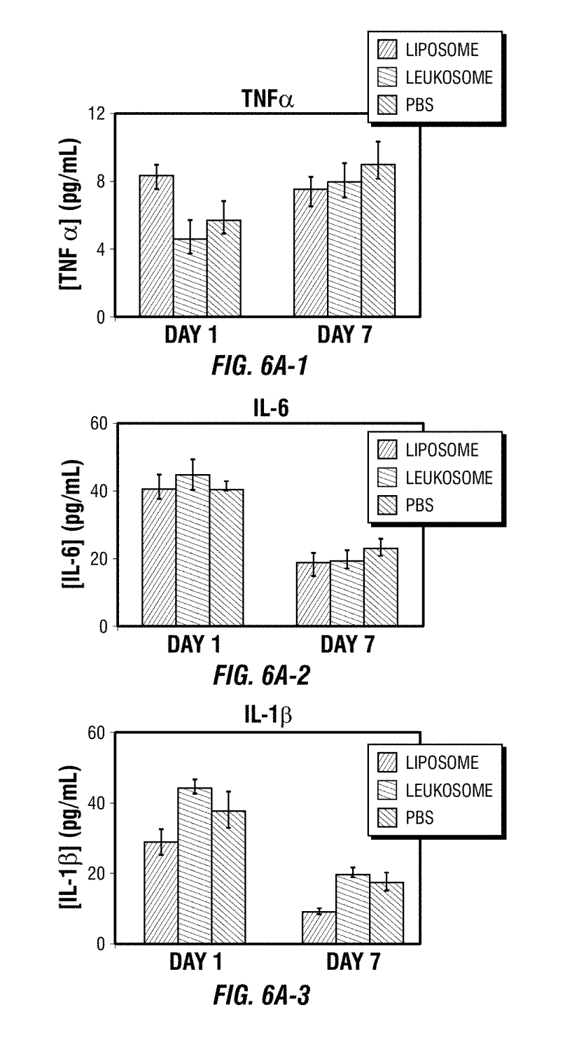

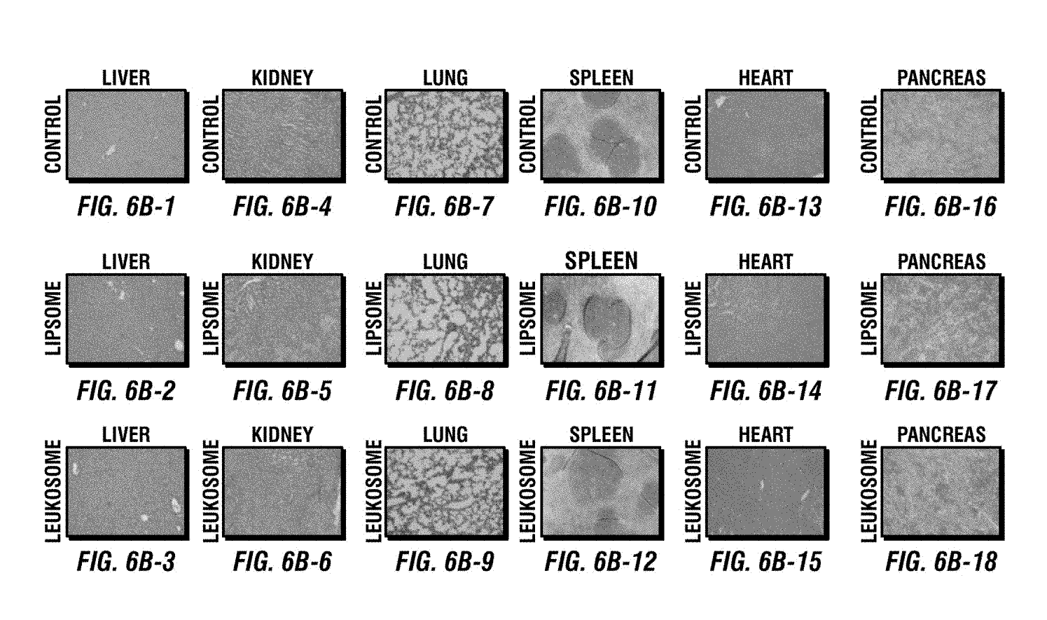

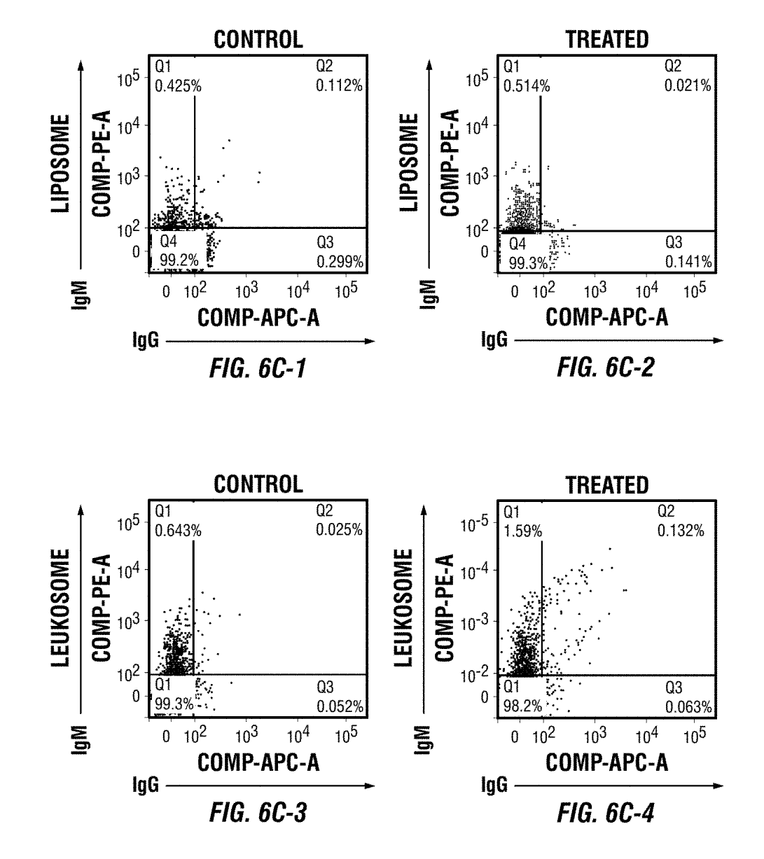

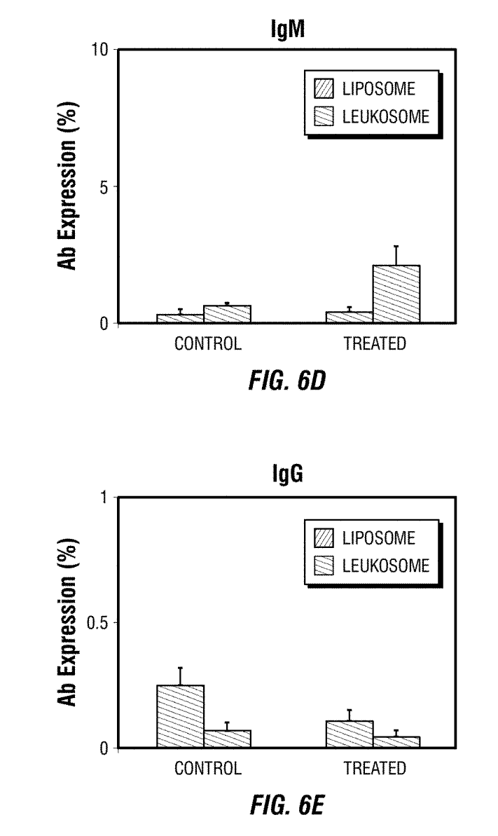

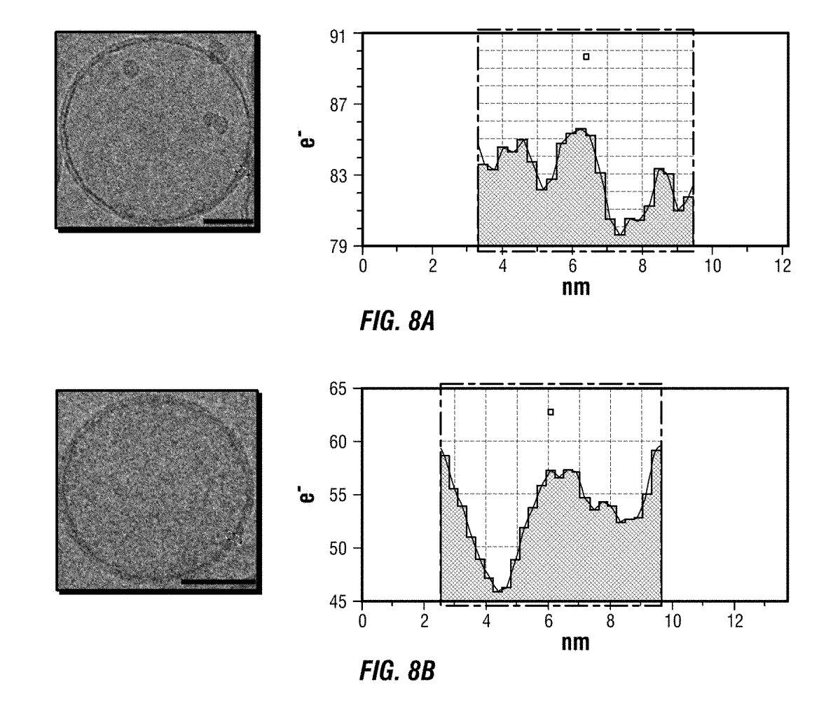

To validate these results in an in vivo model of localized inflammation, lipopolysaccharide (LPS) (10 μg) was subcutaneously injected into the ears of mice. This treatment induces a confined inflammation, manifested by redness, edema, tissue thickening, and neutrophil infiltration, as confirmed by bioluminescence analysis. Being a unilateral inflammatory model, each mouse served as its own control (Gross et al., 2009). Intravital microscopy (IVM) analysis showed a significant increase in the accumulation of leukosomes in the inflamed ear (5-fold and 8.5-fold increase compared to control liposomes at 1 hr and 24 hr after injection, respectively; p<0.1) (FIG. 5A-1, FIG. 5A-2 FIG. 5A-3, FIG. 5A-4, FIG. 5A-5, FIG. 5A-6, FIG. 5A-7, FIG. 5A-8).

Inspection of IVM frames revealed an opposing behavior for liposomes and leukosomes at these two time points. Although leukosomes exhibited a 5- to 8-fold increase in accumulation, liposomes were found more abundant into the extravascular space at 1 hr, possibly because of the enhanced permeability and retention effect occurring at the vascular level after LPS-induced inflammation (Azzopardi et al., 2013). On the other hand, leukosomes were found associated with the inflamed vasculature, due to their active-targeting properties. However, at 24 hr, liposomes were in equilibrium between the two environments, while leukosomes gradually extravasated the vascular barrier and were retained within the extravascular space. These observations led us to believe that, at early time points the accumulation of leukosomes was mediated by the active adhesion to the inflamed endothelium, from where they can achieve extravasation into the parenchyma at later time points. Liposomes, instead, passively distribute only depending on flow dynamics. To dissect key molecules involved in the mechanism of targeting of leukosomes, both in vitro and in vivo adhesion of leukosomes to the inflamed endothelium upon blocking the activity of specific markers was investigated. LFA-1 and CD45, which have direct (Ishibashi et al., 2015) and indirect (Arroyo et al., 1994) roles, respectively, on the binding ability of leukocytes' to the endothelium, were selected. A significant reduction of leukosome adhesion to inflamed endothelia monolayer was observed after both LFA-1 (p<0.005, anti-LFA-1 leukosomes vs. leukosomes) and CD45 (p<0.001, anti-CD45 leukosomes vs. leukosomes) were blocked on the leukosome surface. In vivo studies further validated these results. Blocking LFA-1 or CD45 resulted in a significant decrease in inflamed ear targeting (p<0.001). This confirms that LFA-1 is largely responsible for the vasculature adhesion (Sigal et al., 2000) of leukosomes and validates previous reports (Arroyo et al., 1994; Lorenz et al., 1993) detailing the role of CD45 in LFA-1 mediated leukocyte adhesion during an inflammatory response.

The accumulation of leukosomes at the site of inflammation, as well as their biodistribution and pharmacokinetics profiles, were also assessed through spectrofluorometric analysis on homogenized tissues. Compared to control liposomes, a significant reduction in the accumulation of leukosomes into MPS organs (2.6-fold decrease in spleen, and 1.5-fold decrease in kidneys, liver, and lungs), as well as a prolonged circulation time (5-fold increase), was observed. In addition, leukosomes showed a 7-fold increase in accumulation into the ear tissue compared to control liposomes, confirming the IVM data.

Next, the ability of leukosomes to reduce the inflammation was evaluated using a previously-described, local inflammatory model. The right ear of mice (n=8) were treated with PBS (as control), empty liposomes and leukosomes, DXM-loaded liposomes and leukosomes (5 mg/kg), and free DXM (5 mg/kg) 30 min after LPS. The macroscopic observation of the ear, showed evident signs of improvement in the mice treated with DXM loaded-leukosomes and, surprisingly, empty leukosomes. In comparison, the rest of the groups continued to show signs of acute tissue inflammation (i.e., presence of prominent edema). H&E staining revealed normal tissue architecture in the groups treated with empty and DXM-loaded leukosomes, while all other groups the induced local inflammation resulted in substantial alteration of the ear's architecture, and increased neutrophil infiltration and edematous transudate (FIG. 5B-1, FIG. 5B-2, FIG. 5B-3, FIG. 5B-4, FIG. 5B-5, FIG. 5B-6, and FIG. 5B-7). In fact, a significant increase (p<0.001) in the thickening of the ear (FIG. 5C) and lower neutrophil infiltration (FIG. 5D-1, FIG. 5D-2, FIG. 5D-3, FIG. 5D-4, FIG. 5D-5, FIG. 5D-6, FIG. 5D-7, and FIG. 5E) was observed in the leukosome-treated groups. These findings confirmed the data obtained with H&E analysis, and uncovered a very exciting future role of nanoparticles capable of modulating the inflammatory response because of their intrinsic activity and not solely relying on their therapeutic payload.

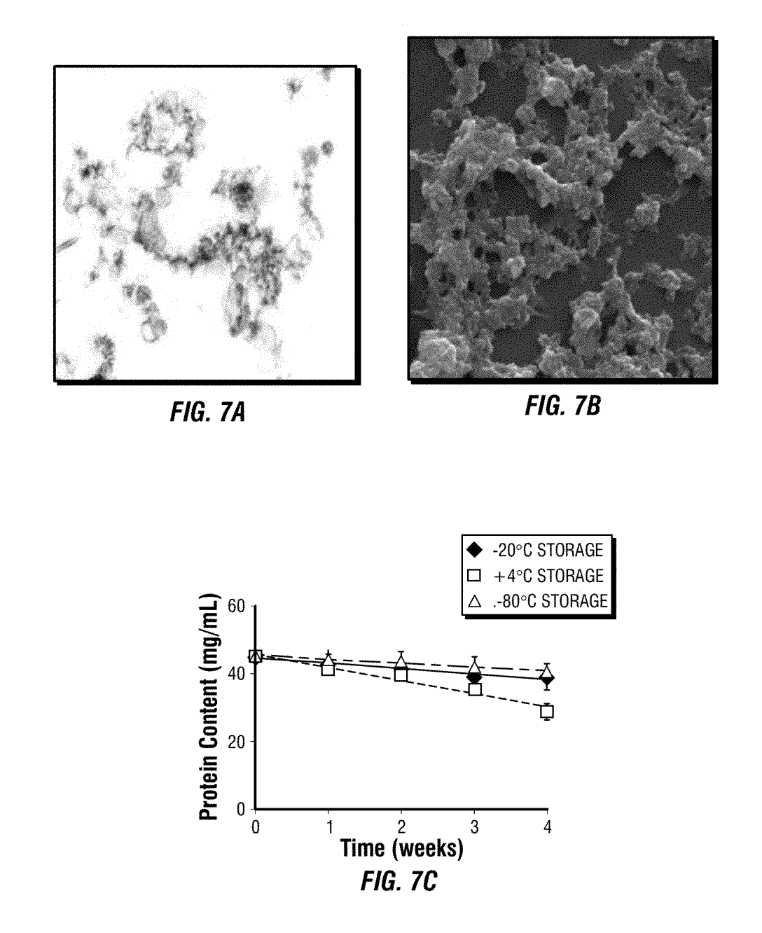

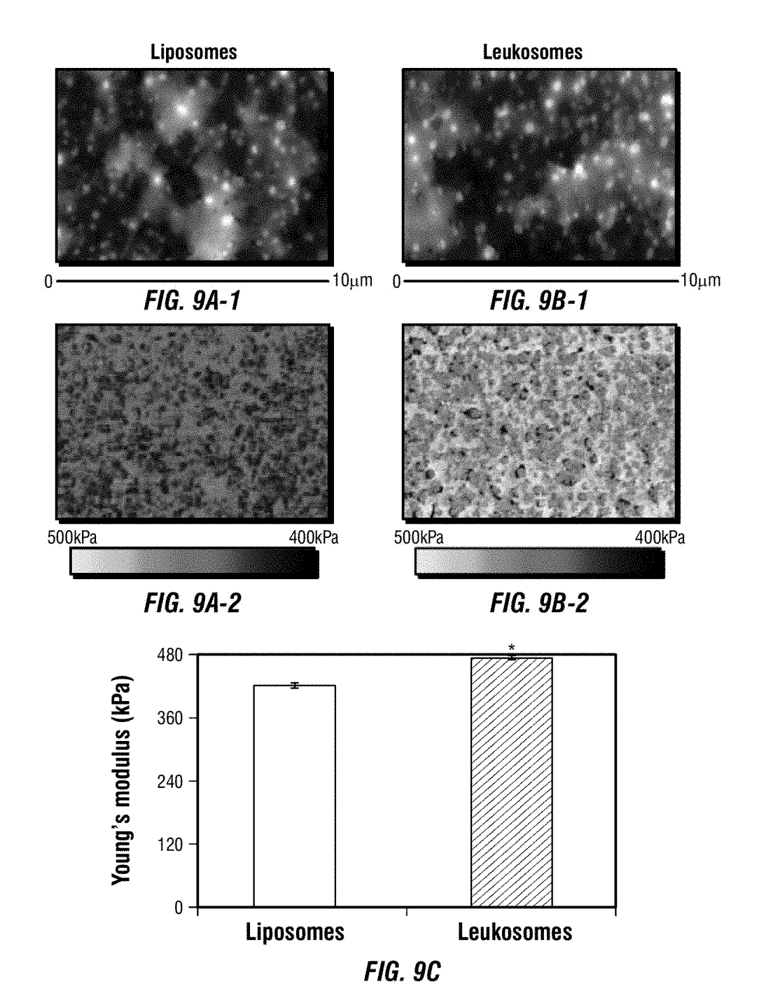

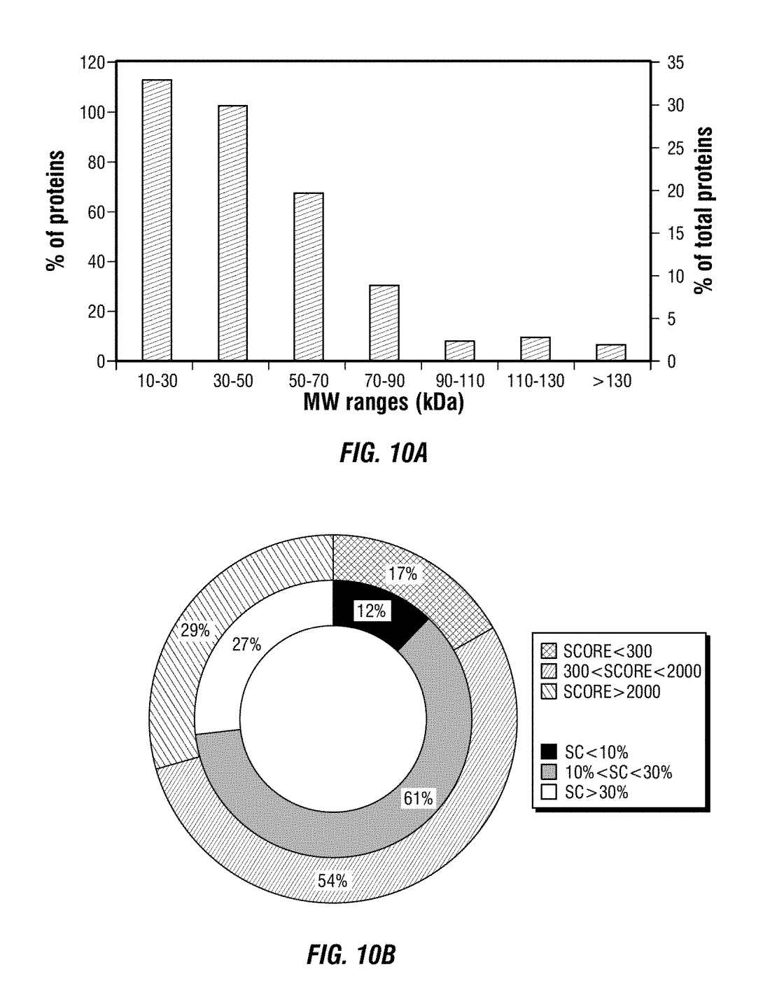

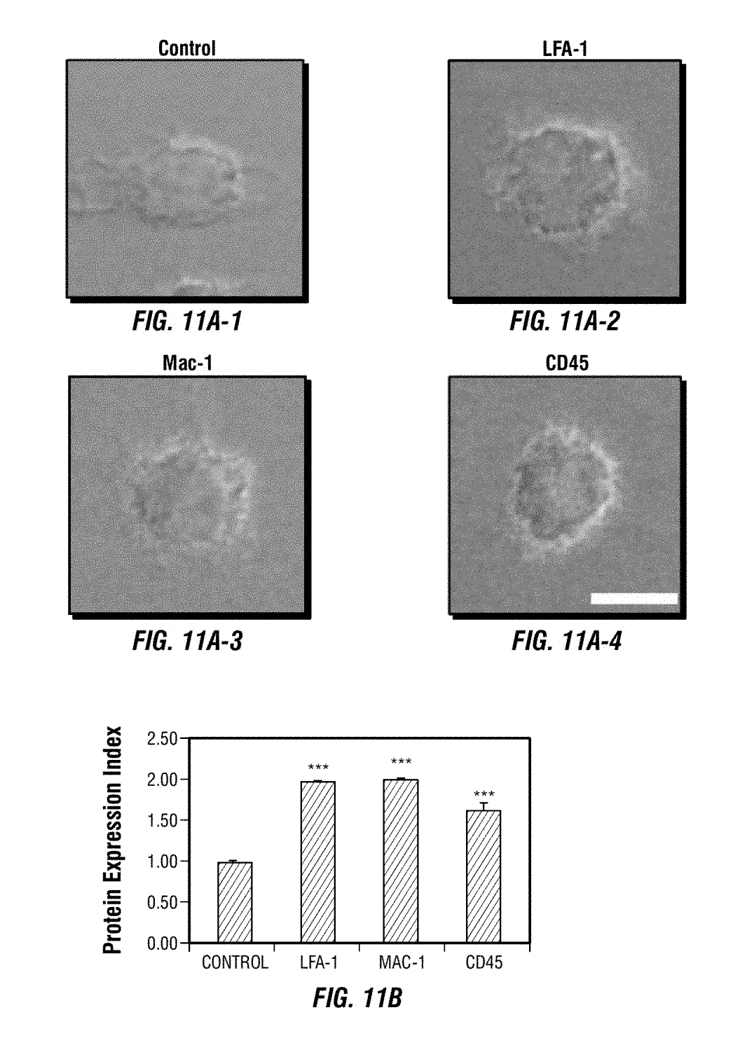

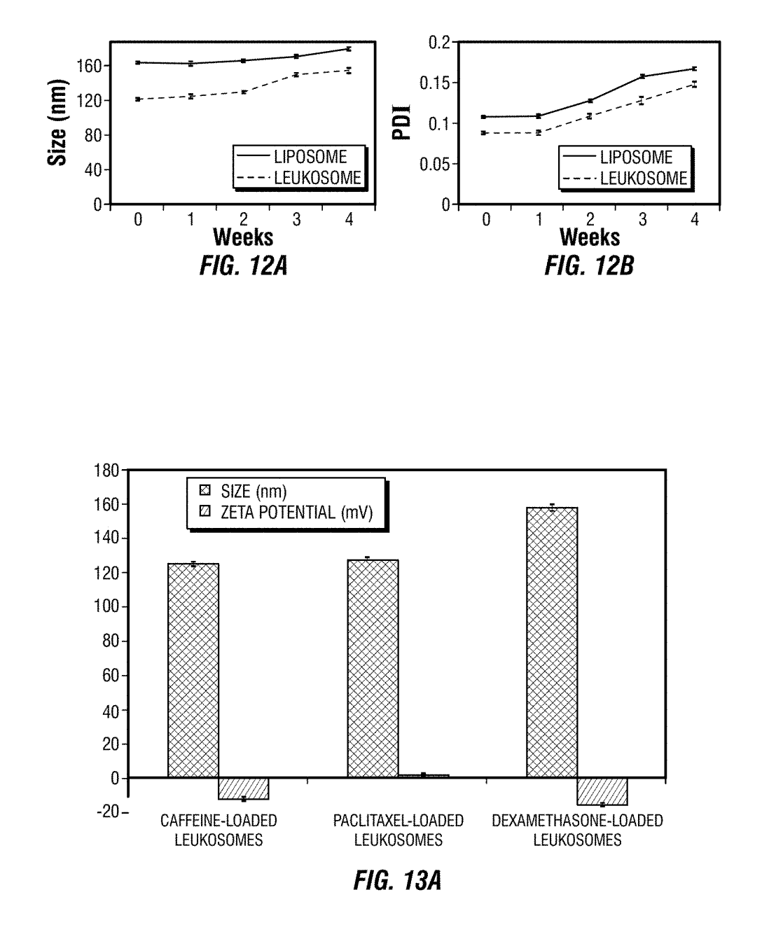

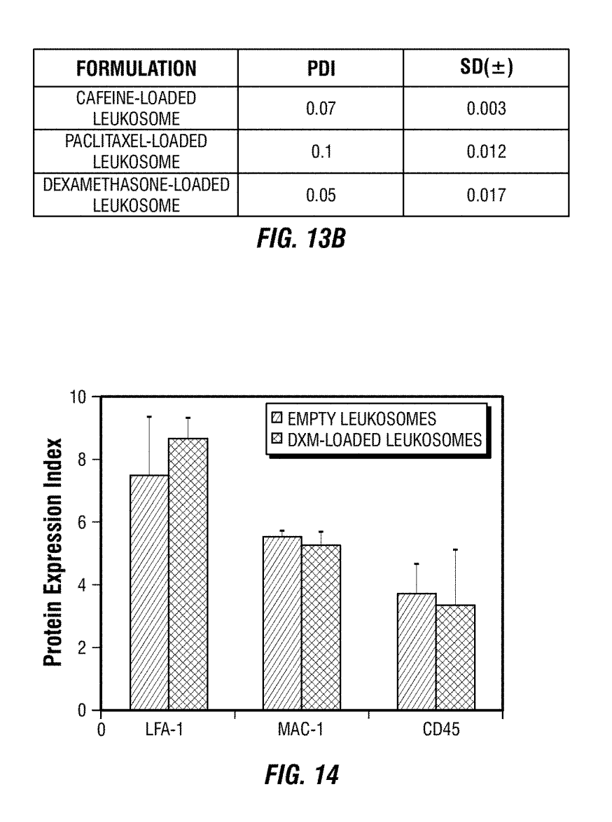

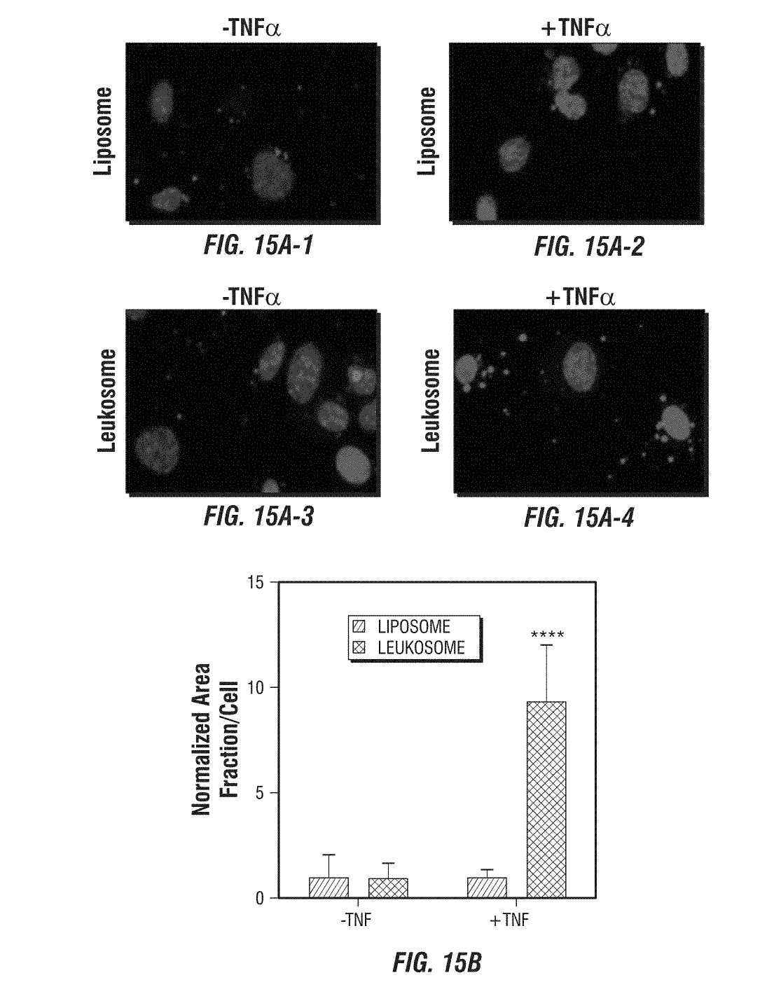

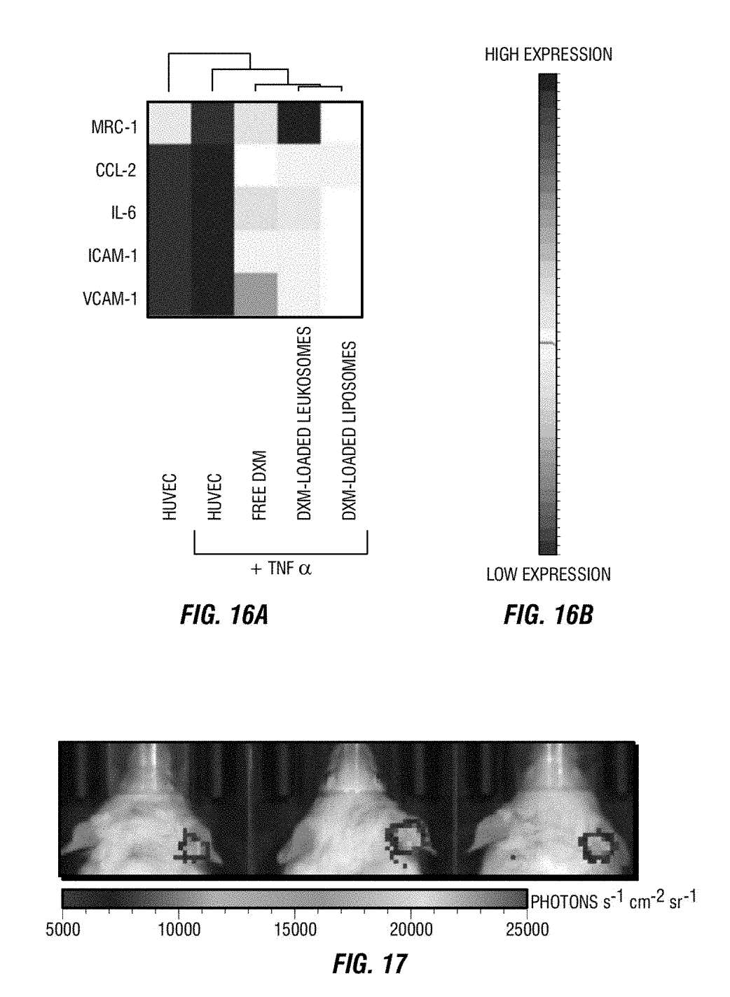

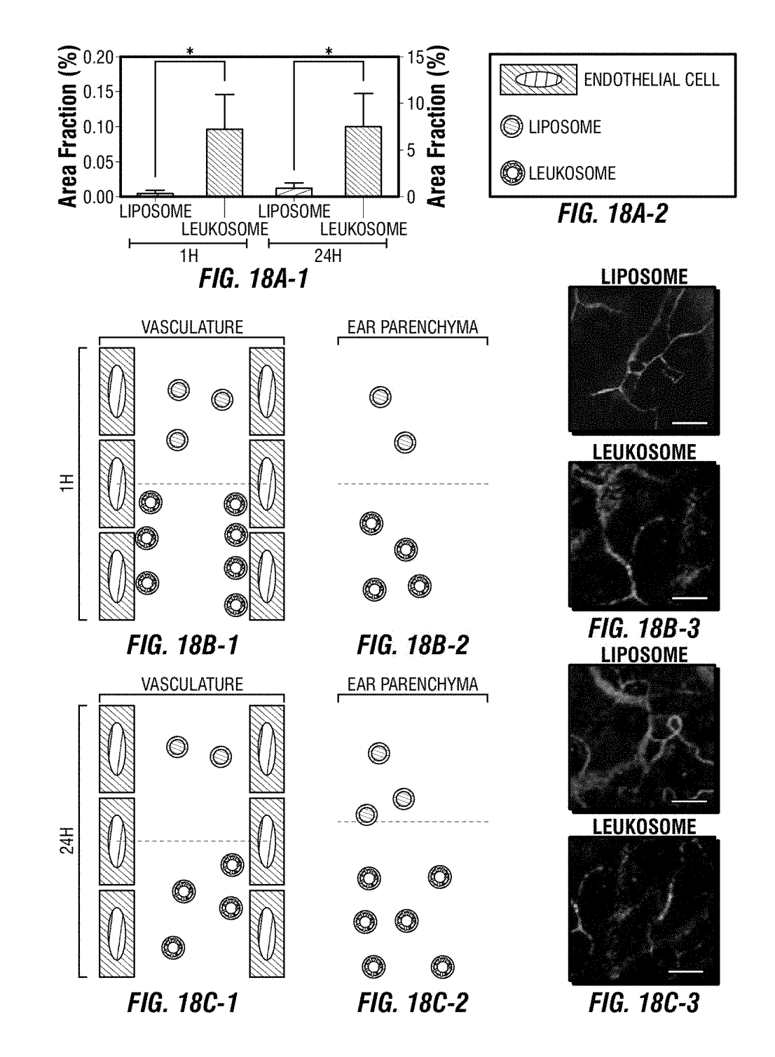

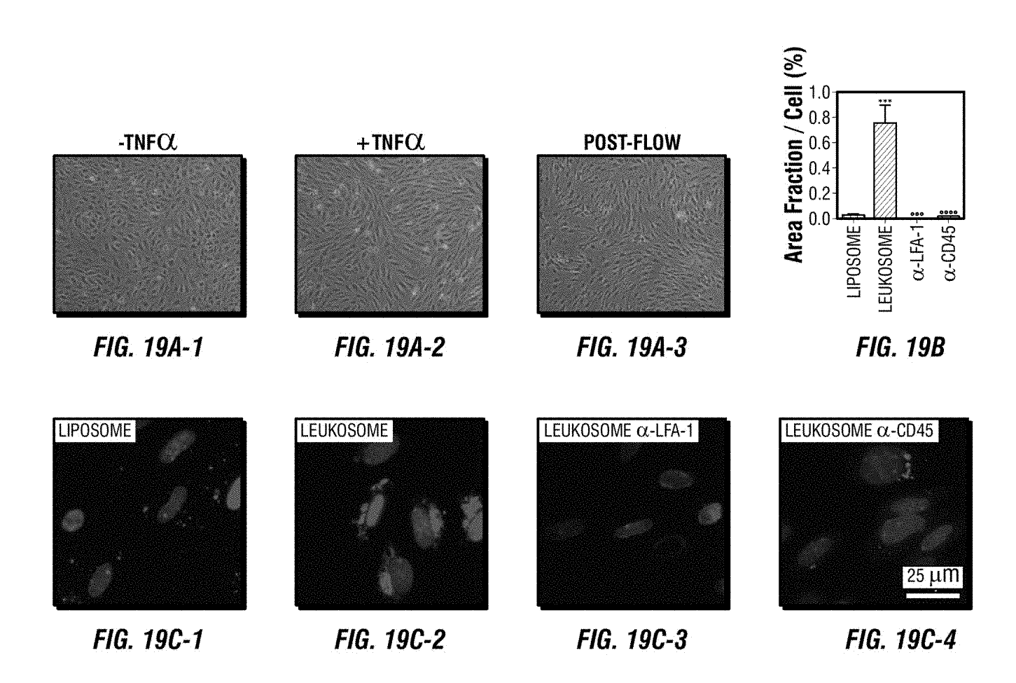

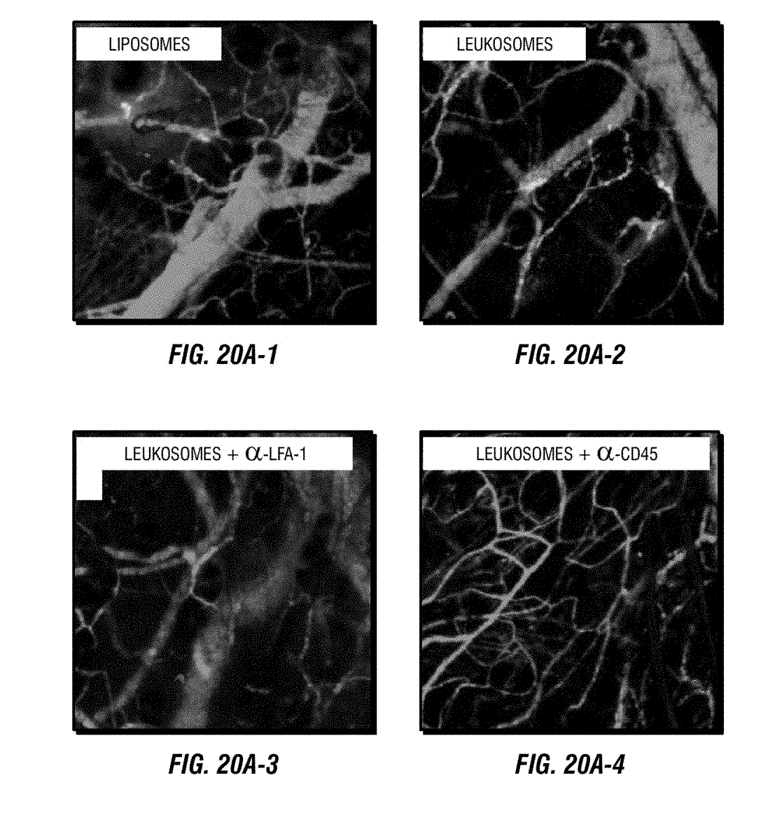

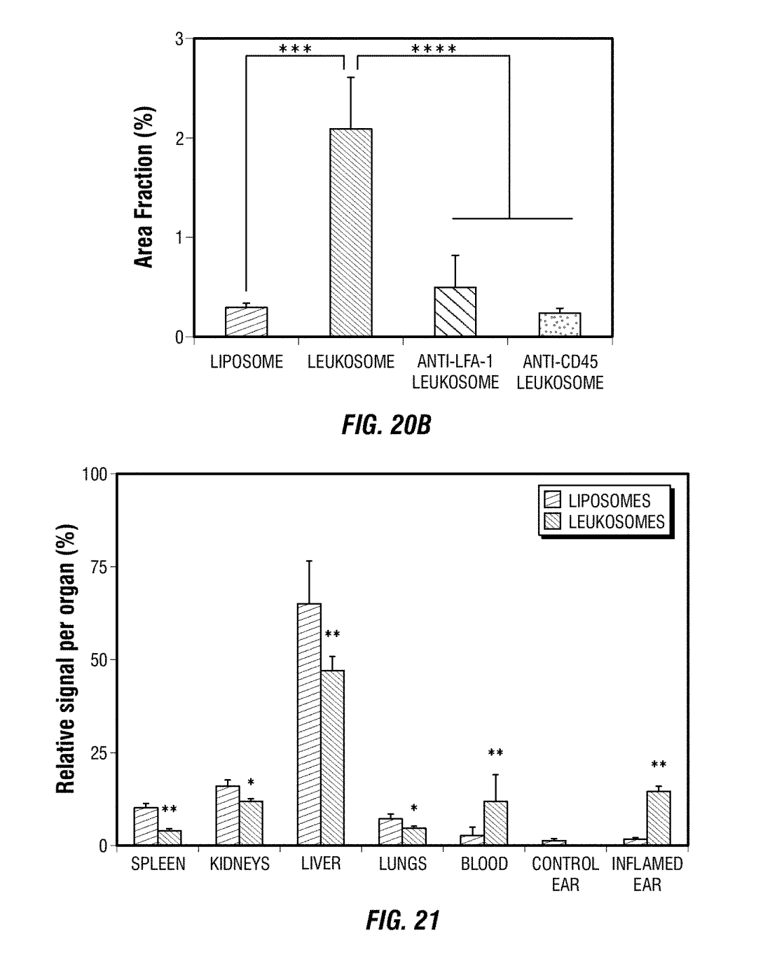

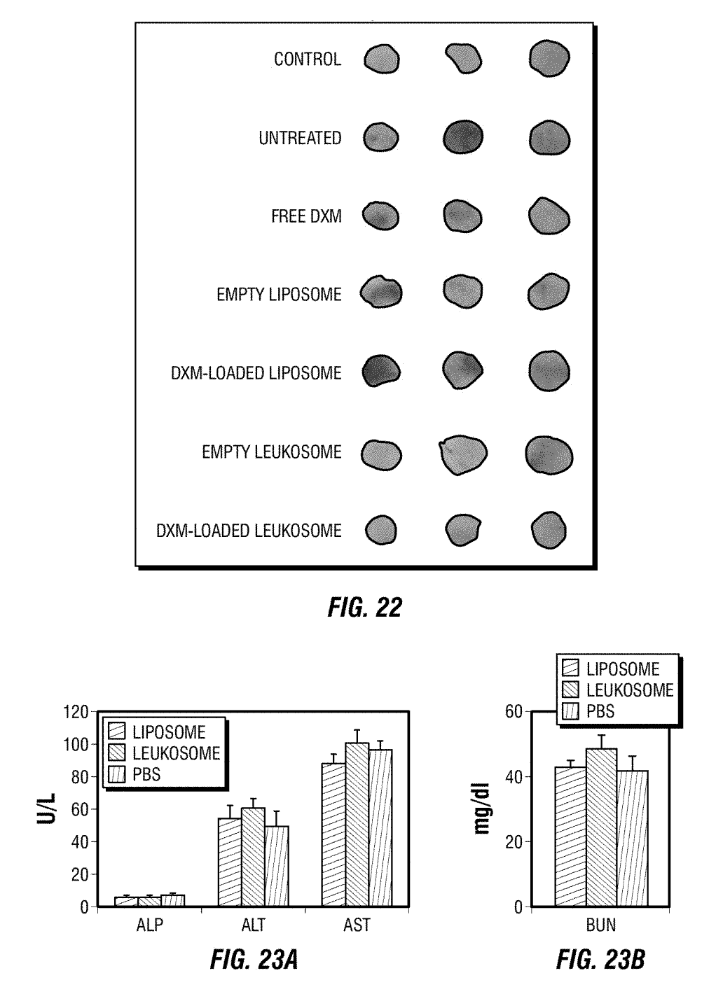

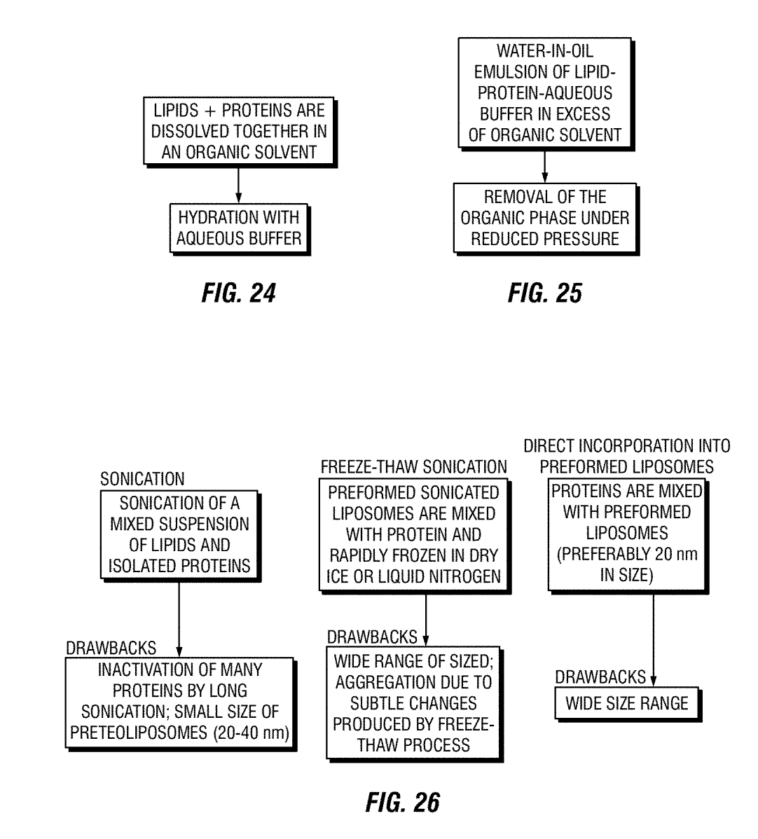

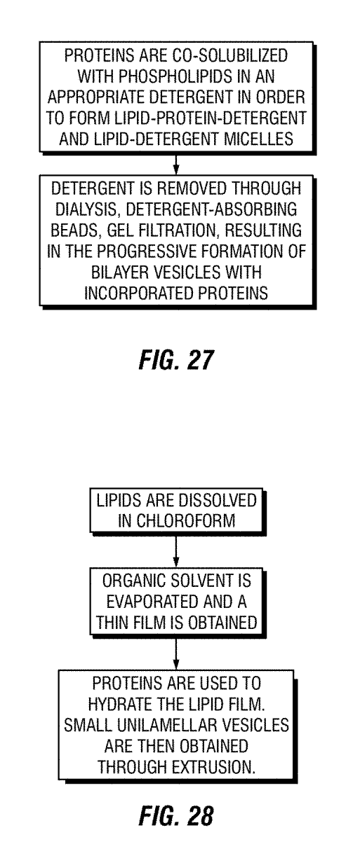

A primary requisite of drug delivery platforms is the in vivo assessment of their safety and biocompatibility. The inventors evaluated whether the systemic administration of a high dose of leukosomes (1,000 mg/kg) would trigger an inflammatory response. Serum levels of cytokines (IL-6, TNFα, and IL-1β) were observed after 1 and 7 days with no significant difference found between leukosomes and the control group (FIG. 6A-1, FIG. 6A-2, FIG. 6A-3). Furthermore, histological analysis on liver, kidney, lung, spleen, heart, and pancreas demonstrated negligible microscopic changes in organ architecture after 7 days (FIG. 6B-1, FIG. 6B-2, FIG. 6B-3, FIG. 6B-4, FIG. 6B-5, FIG. 6B-6, FIG. 6B-7, FIG. 6B-8, FIG. 6B-9, FIG. 6B-10, FIG. 6B-11, FIG. 6B-12, FIG. 6B-13, FIG. 6B-14, FIG. 6B-15, FIG. 6B-16, FIG. 6B-17, FIG. 6B-18). In addition, assessment of the major organ functionality of liver (aspartate aminotransferase [AST], alanine aminotransferase [ALT], and Alkaline phosphatase [ALP]) and kidney (blood urea nitrogen [BUN]) biomarkers revealed minimal differences between the groups. Finally, flow cytometry profiles of IgM and IgG-positive liposomes and leukosomes, previously incubated with serum (primary antibody) of untreated (control) and treated mice showed no observable elevation of autologous antibody titer (FIG. 6C-1, FIG. 6C-2, FIG. 6C-3, FIG. 6C-4, FIG. 6D, and FIG. 6E). In particular, compared to IgM-labeled particles, which reflect the amount of low affinity and less specific antibodies generated toward the particles (FIG. 6D), IgG-labeled particles are 10-fold less abundant (FIG. 6E), thus indicating that low amount of highly specific and high affinity IgG antibody was generated against leukosomes. In fact, less than 3 and 0.3% of leukosomes were labeled by host serum and secondary antibodies for IgM and IgG, respectively, with the same trend observed with control liposomes (FIG. 6C-1, FIG. 6C-2, FIG. 6C-3, FIG. 6C-4, FIG. 6D, and FIG. 6E).

These results suggest that leukosomes do not initiate any significant adaptive immune response or antibody production against membrane antigens related to the leukosomes.

Summary

In the past decades the development of bio-inspired platforms have largely concentrated on two strategies: bottom-up such as surface functionalization with antibodies that mimic original cell surface proteins (Robbins et al., 2010; Chen et al., 2011); and top-down, such as cell-derived nanovesicles and nano-ghosts (Hu et al., 2011; Toledano-Furman et al., 2013). Compared to these strategies, the strength of leukosome relies on i) the high complexity of its surface, faithfully obtained through a facile one-step process that does not require any chemical synthesis or complex purification processes, that are typically required for other organic/inorganic-based systems; ii) the versatility in formulation and application typical of liposomes, such as their capacity to load, retain and release a cadre of different payloads; iii) the standardization of the manufacturing process and the stability of the final product. In addition, leukosomes retained the physiological tropism of leukocytes toward inflamed vasculature and promoted the preferential accumulation into inflamed tissue, the reduction of neutrophils infiltration, and the prevention of tissue damage yielding resolved inflammation.

Physicochemical characterization of leukosomes was performed to evaluate size, zeta potential (ZP) and polydispersity index (Pl), as well as to get structural information before and after the incorporation of the membrane proteins though cryo-TEM analysis. Information about stability in storage condition, loading capacity and release profiles were also collected. Dynamic Light Scattering (DLS) analysis was carried out to evaluate size, Pl and ZP of leukosomes; their size was determined to be around 120 nm, while their surface charge was around −13 mV. The TLE method furnished a homogeneous formulation, as confirmed also from both DLS and cryo-TEM analyses. High-resolution cryo-TEM images revealed that leukosome bilayer is thicker than the liposomal one, probably due to the presence of the transmembrane proteins, as showed by proteomic analysis. In particular, leukosome bilayer thickness is 1.6 folds higher than the liposomal one. AFM analysis showed that leukosomes have a spherical shape similar to the liposome control, but the presence of the protein wrinkles the surface of the particles increasing the surface roughness. Finally, Fourier transform infrared (FTIR) analysis showed how protein insertion modulates leukosome bilayer IR profile. In particular, spectroscopy analysis confirmed the chemical signature of leukocyte cell membranes (black line) embedded within leukosomes.

This approach represents the first time that such a complex material as the plasma membrane is formulated into a lipid vesicle, using the established TLE method, commonly used to synthesize liposomes, to exploit the incorporation of membrane proteins into a lipid bilayer. In addition, the combination of cell biology and nanotechnology opens the possibility of using the plasma membrane of virtually any type of cell for the development of biomimetic particles, or the association of different cellular types (leukocytes, red blood cells, platelets) to create chimeric leukosomes that exploit various intrinsic features to exert their drug delivery function. Finally, the high versatility of this approach enables the leukosome to be used as a technological platform for diagnostic and therapeutic applications suitable for a broad range of disorders that have low therapeutic alternatives (e.g., rheumatoid arthritis, cancer, inflamed bowel diseases) but share the same inflammatory background.

PEGylated liposomes are able to accumulate into tumor tissue due to EPR effect. However, they still present some shortcomings derived from their inability to simultaneously avoid the sequestration by the Reticulo Endothelial System (RES) and efficiently target the therapeutic site, as well as they raised immunological concerns. With the development of the Leukosome both the RES clearance from circulation and targeting of the tumor inflamed microenvironment have been solved.

Lastly, being formulated from the patient's own cells, the leukosome can be considered the ultimate personalized treatment as it could be tailored to the individual needs by fine tuning its composition, formulation and source of cell membranes.

Several clinical problems spanning from bacterial infections to tumor neoangiogenesis involve inflammatory processes, which are the key targets of the leukosome. The development of a new generation of humanized liposomes provides unprecedented intravascular abilities. The leukosomes descirbed herein are ideal for addressing an array of clinical applications due to their inflammation homing properties and payload carrying characteristics. For instance, Leukosomes formed with cationic lipids are ideal for carrying siRNA or other kinds of genetic material.

The disclosed leukosomes represent the first hybrid drug delivery system. Protein inclusion into liposomal bilayer has been extensively studied and reported in literature, but with the only aim to study their behavior in a simplified platform. In addition, the TLE method used here to assemble leukosomes, has been used in the past, but only for the encapsulation of hydrophilic drugs, included proteins, into liposome aqueous core and subsequently deliver them for different purposes. In this protocol, the protein-to-lipid ratio necessary for the right assembly (without the risk of triggering any immunogenic reaction once leukosome is administered) was determined, which supported this datum by experimental proof. This method, coupled to the protein extraction, permits guiding of the fusion process between membrane proteins and phospholipid bilayer, so using proteins to bio-activate liposomal surface. The advantages of the present invention consist in the high scale up of this platform to industrial production, thanks also to high availability of components and the very low complexity of the synthetic process. In fact, protein enrichment is the result of a one step synthesis without the involvement of any chemical reaction or post-synthesis modification. All these features are going to simplify FDA approval and its translation to the clinic.

### Example 2—Co-Encapsulation of Top2 Poisons and Tdp2 Inhibitors in Leukosomes to Improve Cancer Treatment Options

Top2 (Type II topoisomerase) poisons like Etoposide are part of common core of many chemotherapeutic regimes and kill cancer cells by inducing Top2-DNA covalent complexes that cause cell death by preventing DNA synthesis. Although Top2 poisons are effective, cancer cells often show resistance. Recent discovery of Tyrosyl DNA Phosphodiesterase 2 (TDP2) offers one potential basis for Top2 resistance in cancer cells where TDP2 helps to remove the Top2-DNA adducts, thus enhancing resistance to Top2 poisons. TDP2 overexpression in cancer cells should allow TDP2 inhibitors to be effective as an adjuvant to Etoposide for overcoming resistance and enhance cancer specific cell killing. Additionally adverse systemic toxicity of Etoposide can be reduced by targeted delivery and release of drugs to tumor endothelial cells via uniquely formulated nanoparticles, thus further enhancing Top2 poisons therapeutic value.

Glaucine, a known antitussive agent, has been identified as the lead compound based on drugability and TDP2 inhibitory activity through high-throughput screening of a library of 370,226 small molecules. Glaucine was repositioned as a TDP2 inhibitor through multiple assays and its synergy and specificity was shown with etoposide. Taken together, these data incontrovertibly identify Glaucine as the 1″-in-class TDP2 inhibitor able to enhance Etoposide mediated cancer cell killing. However, the efficacy of therapeutic approaches based on drug formulation also depends upon pharmacokinetics, bioavailability and clearance of the active principles. In addition, this therapeutic has been co-encapsulated in the leukosome delivery system described in Example 1 to promote specific accumulation of the drugs at the cancer site, and to minimize the onset of potential side effects on off-target organs.

Materials and Methods

TDP2 inhibitors and Etoposide were co-encapsulated inside the leukosomes according to their solubility. Etoposide (0.5 mg/mL) was dissolved in the organic phase (chloroform:methanol 3:1 (vol./vol.) containing the lipid mixture, which was subsequently dried and hydrated with 1 mL of the aqueous solution containing the TDP2 inhibitors (1 mg/mL). Unilamellar vesicles were obtained by extrusion through 200-nm pore-size cellulose acetate membranes. Raw materials (drug, lipids and membrane proteins) not incorporated into the formulation were eliminated by dialysis with PBS.

Results

The inventors have verified that the selected Top2 poisons synergized with Etoposide in killing human lung cancer cells in vitro. Protocols were optimized to co-encapsulate the different therapeutics in leukosomes, and the release kinetic of different drugs was evaluated. Previous data showed that upon intravenous injection in mice leukosomes significantly target and resides in the lung tissue for several hours. (b) Additional in vivo studies were performed to demonstrate the efficacy and safety of this approach in inhibiting cancer growth and reducing the onset of potential side effects in comparison with typical free administration of the different drugs.

Discussion

Leukosome targeting mechanism is based on the targeting of inflamed vasculature associated with most of the neoplastic lesions, lung cancer included. The unique surface properties of the system consist of isolated cell membrane of immune cells that showed a natural ability to adhere to the endothelial receptor over-expressed in the inflamed tissue. The system can be indifferently loaded with different therapeutics allowing for a homogenous release of the different encapsulated drug. These properties are fundamental in order to potentiate the therapeutic properties of Etoposide with TDP2 inhibitors because they allowed the co-delivery of these payloads in the same site and at the same time.

Furthermore the synergistic effect of selected TDP2 inhibitors has been tested with other Top2 poisons, like doxorubicin, daunorubicin, and various quinolones, and it has been shown that their ability in killing other cancer cell phenotypes can benefit from this treatment. Liver cancer, for example, is currently treated with Top2 poisons, and can be significantly targeted by leukosomes.

With this approach, it is possible to overcome 2 typical limitations that affect the efficacy of current cancer treatments employing on etoposide: 1) overcome possible limitations due to the onset of resistance phenomena related to the acitivity of TDP2; and 2) reduce adverse systemic toxicity related to Etoposide and Etoposide+TDP2 inhibitors by selectively targeting cancer lesions through encapsulation of the therapeutics in leukosomes. In other words, compared to current therapeutic approaches for cancer, the present system enhances the therapeutic properties and the safety of the treatment.

Although Top2 poisons are effective, cancer cells often show resistance and systemic toxicity. By employing leukosome-based delivery systems, chemo-resistance and systemic toxicity can both be reduced.

### Example 3—Preparation of Leukosomes for Targeting Inflamed Tissues

Recent advances in the field of nanomedicine have demonstrated that biomimicry can further improve targeting properties of current nanotechnologies while simultaneously enable carriers with a biological identity to better interact with the biological environment. Immune cells for example employ membrane proteins to target inflamed vasculature, locally increase vascular permeability, and extravasate across inflamed endothelium. Inspired by the physiology of immune cells, we recently developed a procedure to transfer leukocyte membranes onto nanoporous silicon particles (NPS), yielding Leukolike Vectors (LLV). LLV are composed of a surface coating containing multiple receptors that are critical in the cross-talk with the endothelium, mediating cellular accumulation in the tumor microenvironment while decreasing vascular barrier function. The inventors previously demonstrated that lymphocyte function-associated antigen (LFA-1) transferred onto LLV was able to trigger the clustering of intercellular adhesion molecule 1 (ICAM-1) on endothelial cells. The present example provides a more comprehensive analysis of the working mechanism of LLV in vitro in activating this pathway and in vivo in enhancing vascular permeability. The results suggested the biological activity of the leukocyte membrane could be retained upon transplant onto NPS, and was critical in providing the particles with complex biological functions towards tumor vasculature.

The specific targeting of cancer lesion remains the primary goals of nanomedicine applied to oncological disease and represents a promising opportunity to increase poor cancer patient survival. Over the past decades, nanomedicine has provided several delivery platforms demonstrated to enhance chemotherapeutic delivery; however, current results are still unsatisfactory. A significant accumulation in the cancer lesions is hampered by several biological barriers (e.g., mononuclear phagocytic system, tumor-associated vasculature, tumor extracellular matrix, and cellular membrane) standing between the point of administration and the pathological site. The ideal treatment should be able to overcome each of these barriers in a sequential manner to reach its intended site. The successful negotiation of tumor-associated vasculature represents one the greatest challenges in improving the effectiveness of current treatments and diagnostic tools.

Previously, nanocarrier accumulation relied on exploiting the superior permeability of tumor vasculature, a phenomenon commonly referred to as the enhanced permeability and retention (EPR) effect. Further understanding of the ultrastructure and transport that occurs in cancer lesions allowed for the rational development of carriers that specifically target diseased tissue by exploiting lesion-specific transport oncophysics. On the other hand, a better understanding of the biological features characterizing tumor blood vessels highlighted the possibility to design carriers with biological properties, prompting a deeper investigation into alternative vector-associated modifications and tumor characteristics. In particular, cancer associated inflammation and tumor vasculature provides several opportunities to develop targeted therapies by leveraging the adhesive proteins over-expressed on inflamed vessels.

The inventors recently developed a technique for functionalization of the surface of nanoporous silicon particles (NPS) with purified leukocyte membranes. These NPS were shown to be biocompatible, degradable, and able to be rationally designed in order to cross a multiplicity of sequential biological barriers to attain preferential concentration at desired target cancer locations. These NPS formed the asis for multi-stage vectors and injectable nanoparticle generators for the cure of visceral metastases in triple-negative breast cancer. The functionalization of NPS with purified leukocyte membrane was demonstrated on select variants of the NPS platforms, yielding leukolike vectors (LLV), which displayed properties similar to their leukocyte source while preserving some favorable properties of NPS (e.g., drug loading and release, margination) on those select variants. Specifically, LLV were demonstrated to be successfully functionalized with more than 150 leukocyte membrane-associated proteins, including adhesive surface proteins involved in leukocyte diapedesis and were shown to efficiently interact with intercellular adhesion molecule-1 (ICAM-1) inducing its clustering. ICAM-1 is overexpressed in tumor-associated vasculature and is involved in leukocyte adhesion and endothelial reorganization. This process is critical in mediating vascular permeability as a result of decreased expression of endothelial intercellular junctions at the endothelial cell border, thereby favoring immune cell infiltration. In this example, it is demonstrated that the cell membrane applied on the surface of synthetic NPS remained functional in triggering the biomolecular events that culminate in increased vascular permeability. In addition, the coating was s also shown to maintain its biological properties in vivo, favoring LLV firm adhesion on tumor-associated vasculature and resulting in increased perfusion of small molecules into the subendothelial space. Importantly, these data definitively proved that specific biological activities that characterize the surface of leukocytes can be transferred onto synthetic carriers, providing them with a biological identity and favoring their molecular interaction with vascular tissue both in vitro and in vivo.

Materials and Methods

Leukolike Vector Fabrication.

NPS were fabricated at the Microelectronics Research Center at The University of Texas at Austin (Austin, Tex., USA). APTES-conjugation was performed by mixing oxidized NPS in a solution containing 2% APTES (Sigma-Aldrich, St. Louis, Mo., USA) and 5% MilliQ water in isopropyl alcohol and mixed under continuous and constant agitation for 2 h at 35° C. After incubation, particles were washed three times in isopropanol and stored in IPA at 4° C. Fluorescent labeling of NPS was achieved by mixing them in a 100 mM triethanolamine (in DMSO) solution containing AlexaFluor 488 or 555 (1 mg/mL, Life Technologies) for 2 hrs at room temperature under brief agitation. NPS were then washed to remove free dye and stored at 4° C. in isopropyl alcohol.

The LLV were fabricated following protocols previous established by our group. Cell membranes were isolated by brief homogenization in a Dounce homogenizer and spun down at 500×g for 10 min at 4° C. The supernatant was collected and pooled after three additional homogenization steps. The pooled supernatant was placed on a discontinuous sucrose gradient (55-40-30% wt./vol. sucrose) and centrifuged at 28,000×g for 45 min. The membrane at the 30-40 interface was collected and washed again in 150 mM NaCl solution. It was then mixed with APTES-conjugated NPS using a 1.5:1 (membrane:particle) mass ratio and incubated overnight under continuous rotation at 4° C. Unbound membranes were then washed using 150 mM NaCl solution by centrifugation using a setting of 750×g for 10 min. Dynamic light scatter and Zeta potential were performed by suspending 10⁷ particles in MilliQ water and measured for the particle size using a Zetasizer Nano ZS (Malvern, Malvern, UK). The sample was then placed into a disposable folded capillary cell and zeta potential was measured. Jurkat cell membranes were used for in vitro studies while J774 cell membranes were used for in vivo studies.

Flow Cytometry.

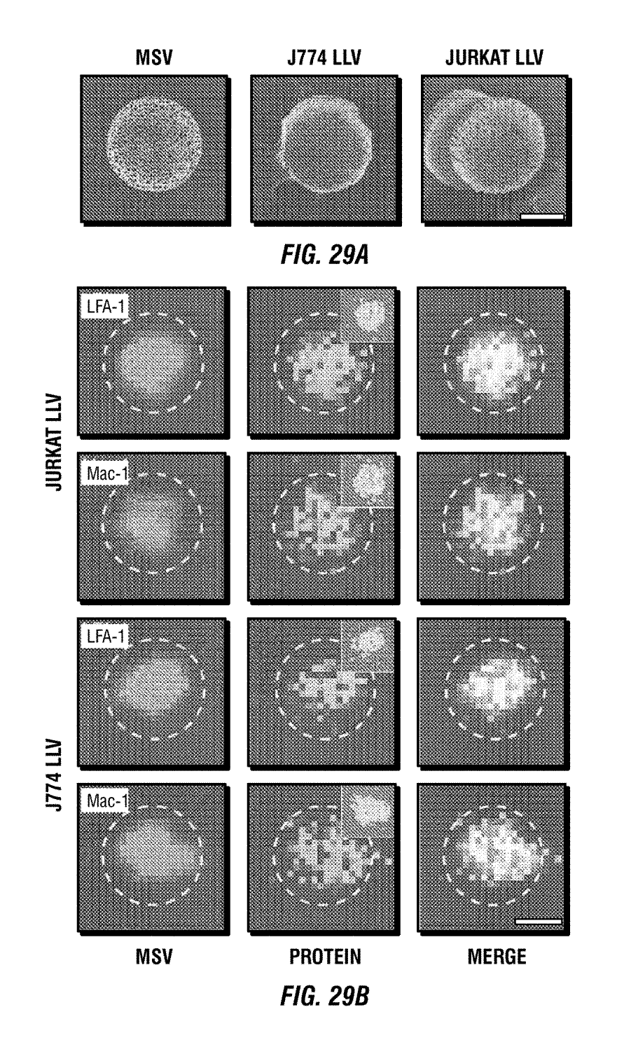

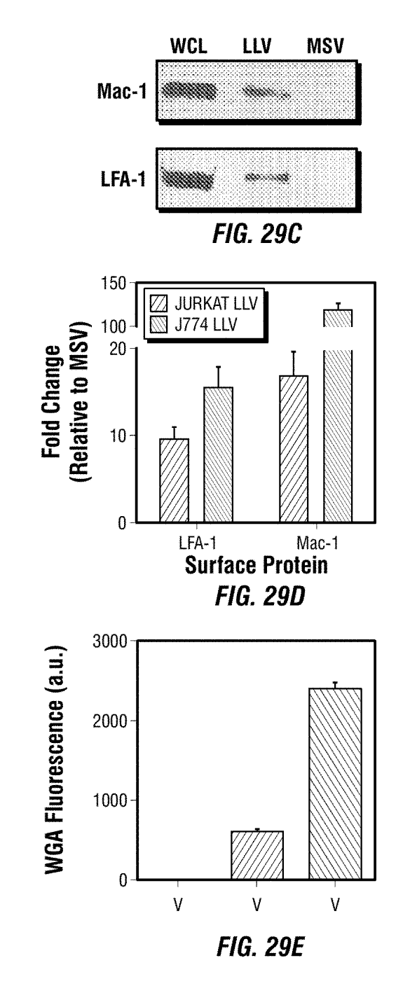

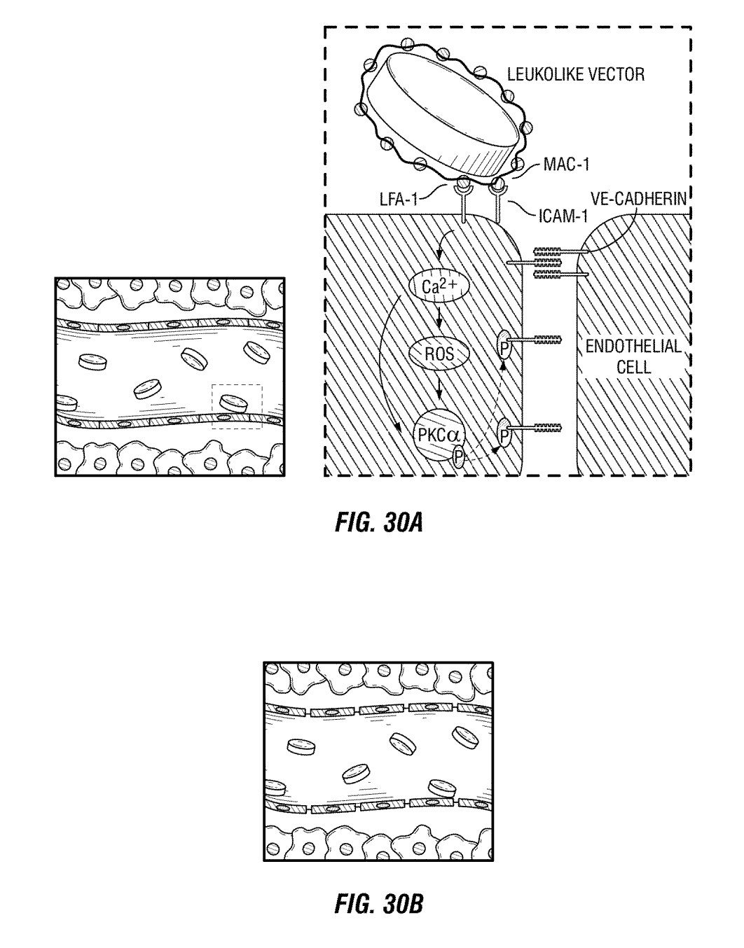

Surface proteins were quantified by mixing 5×10⁶ particles in a FACS buffer solution (1% bovine serum albumin, BSA) blocking solution for 30 min. Next, particles were washed and allowed to mix with FITC Rat Anti-Mouse CD11a (LFA-1) or Alexa Fluor® 488 Rat Anti-Mouse CD11b (Becton Dickinson, Houston, Tex., USA) suspended in FACS buffer at a concentration of 0.5 m/mL for 1 hr. After incubation, unbound antibodies were removed by three washes in FACS buffer and centrifugation at 450×g for 10 min. Samples were analyzed by collecting a minimum of 5,000 events using a BD LSR Fortessa (Becton Dickinson) cell analyzer equipped with BD FACS Diva software (FIG. 29D).

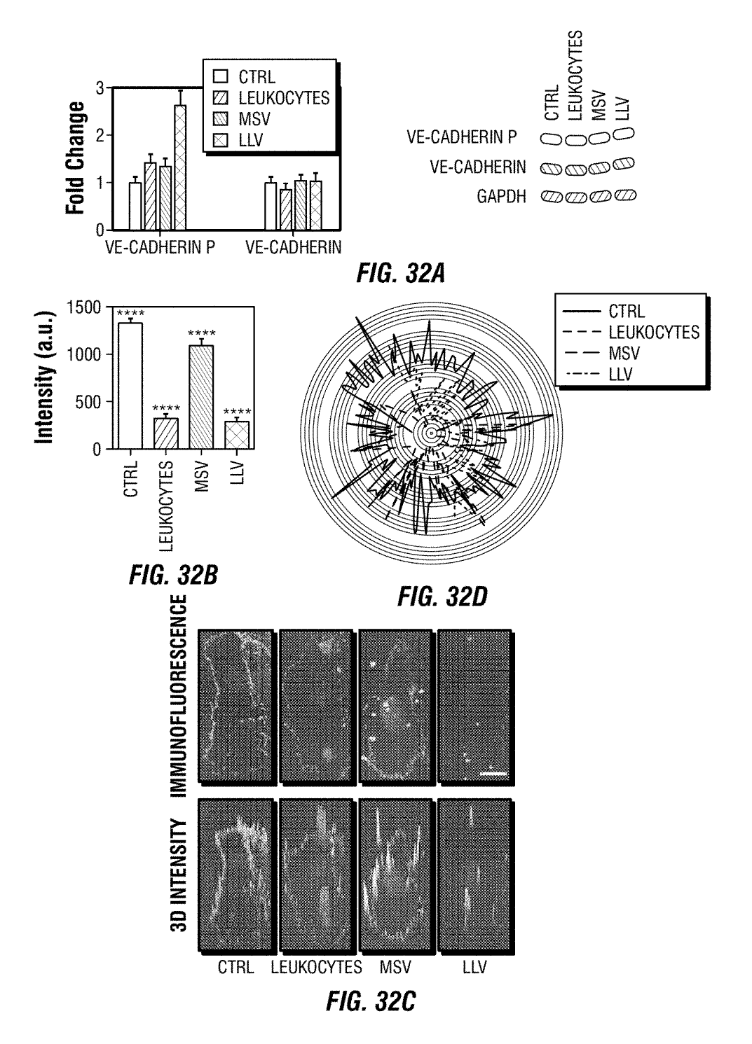

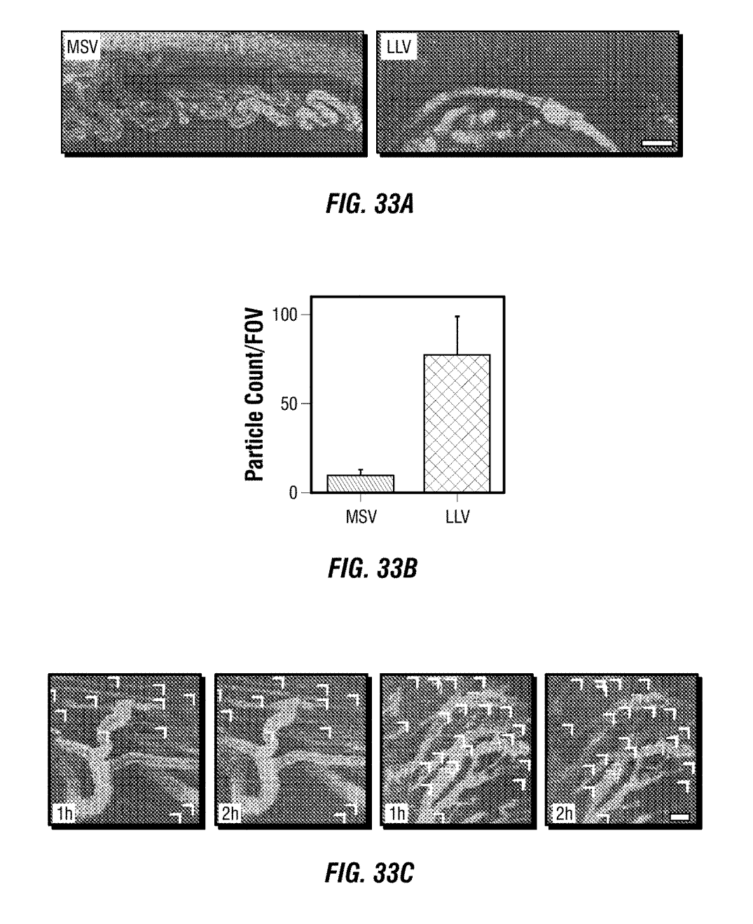

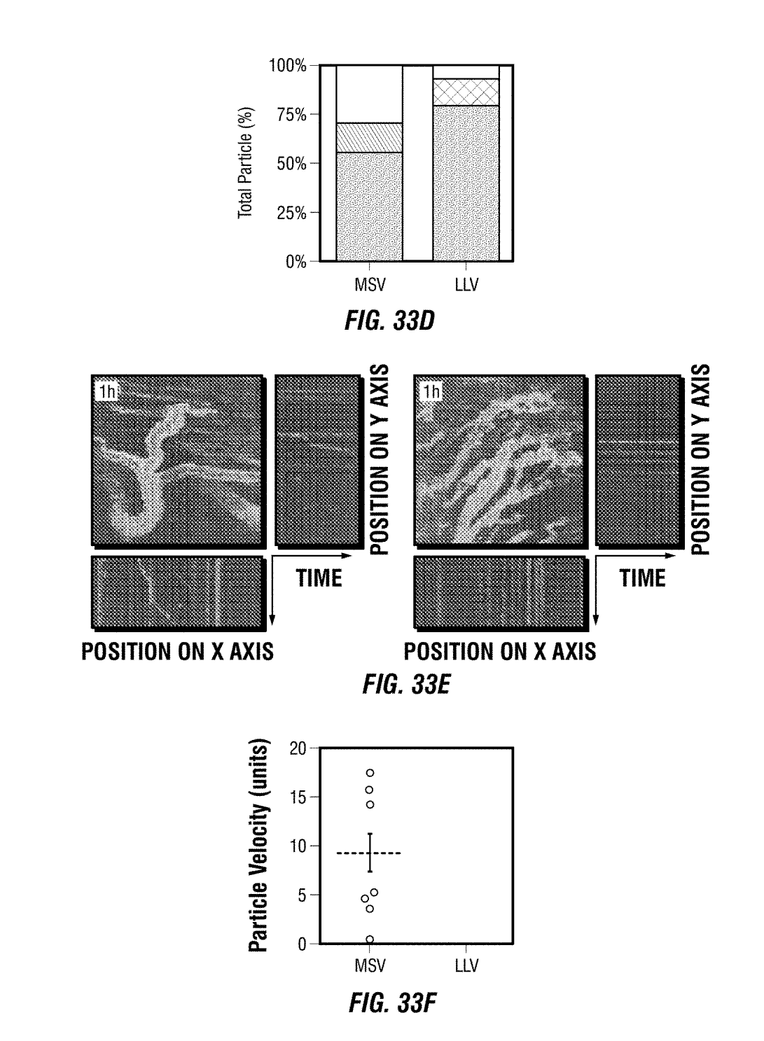

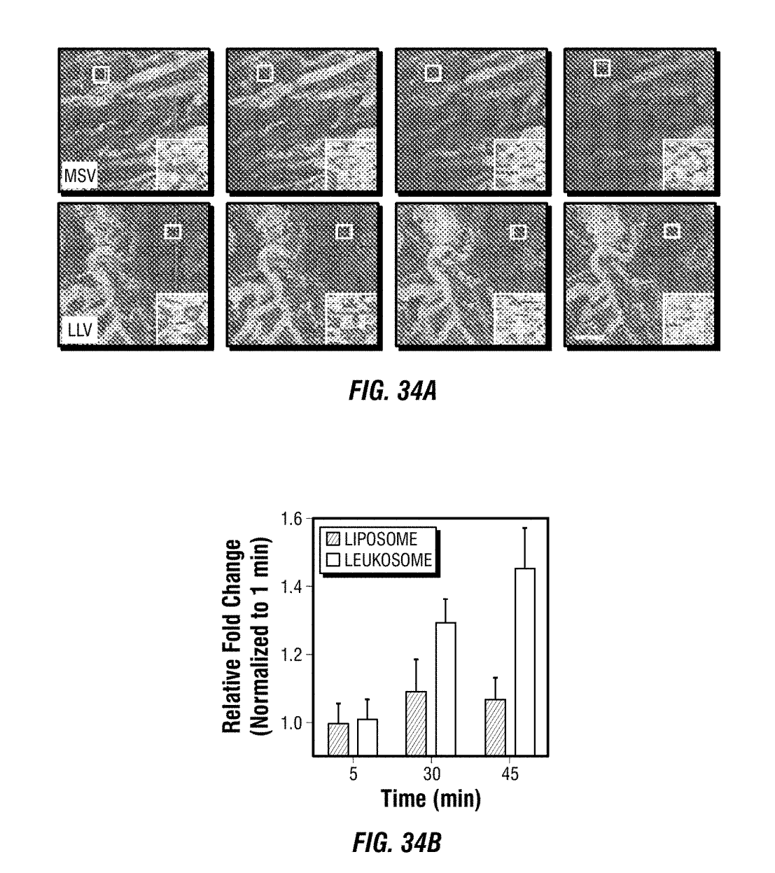

Particle and cell flow experiments. 3×10⁵ HUVEC cells were seeded on fibronectin-coated flow cells (0.4 Ibidi μ-slide; IBIDI, Planegg/Martinsried, Germany) in media with or without TNFα (25 ng/mL). Twenty-four hours later, 3×10⁷ NPS, or LLV, or 3×10⁵ Jurkat cells (indicated as “leukocytes” in the following Results section) were introduced into the flow cell at a rate of 0.1 dyn/cm² for 30 min. Cells were subsequently fixed and prepared for microscopy as described below (FIG. 33C). We used the same conditions for live microscopy experiments. Intracellular Ca²⁺ levels were monitored using Fluo-3/AM, Calcium Indicator (Life Technologies) according to the vendor's specifications (FIG. 32C).

Immunofluorescence.

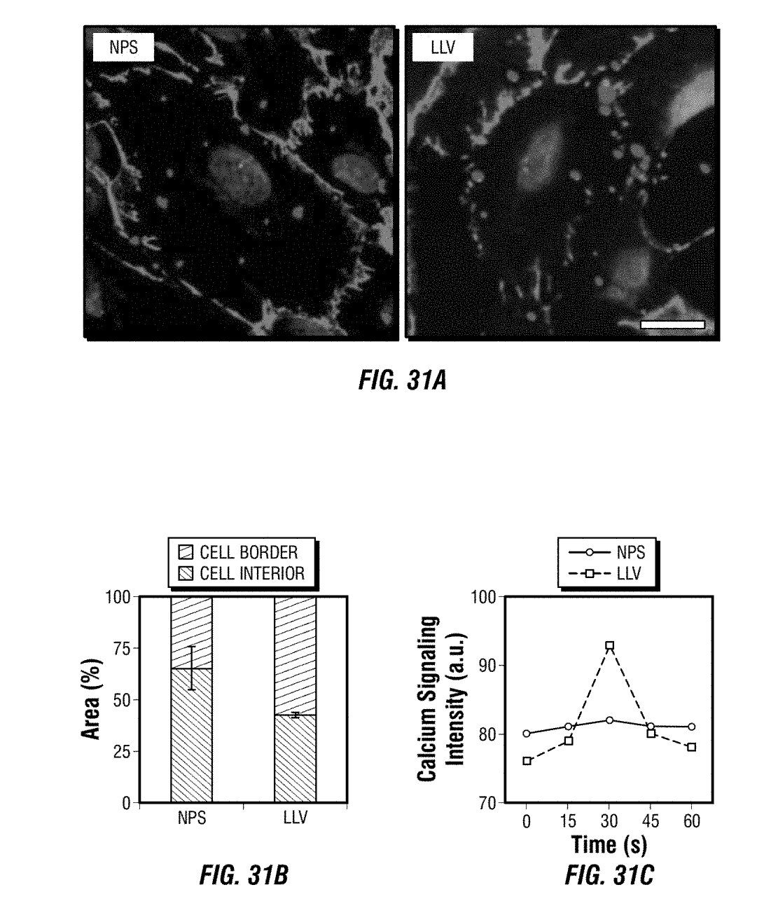

After particle flow (see above), cells were fixed with 4% PFA, washed twice with PBS 1%-BSA 0.2%-Triton for 5 min. Before and after hybridization with the primary antibody (anti-VE-cadherin Ab-33168 “Abcam”-Cambridge, UK) cells were washed with PBS 1%-BSA. Secondary antibody hybridization was performed using Alexa Fluor® 488 labeled anti-rabbit (Thermo Scientific, Waltham, Mass., USA). Nuclei were stained using DAPI (FIG. 31A and FIG. 31C). Images were taken using an Inverted Nikon FLUO-Scope (Nikon, Tokyo, JAPAN). Data were analyzed with Nikon software ND2. For particle immunofluorescence, samples were prepared as described above for flow cytometry. After particle conjugation with antibody, 10⁵ particles were seeded on an 8-well Nunc® Lab-Tek® Chamber Slide™ (Thermo Scientific). Images were acquired with a Nikon A1 confocal imaging system and analyzed with Nikon NIS Elements software (Nikon).

Western Blot Analysis.

Whole cell lysate from Jurkat cells and HUVECs were used in this study. Cells were washed with PBS twice and collected by centrifugation at 125×g for 10 min. Cells were resuspended in RIPA buffer (5 mM Tris-HCl (pH 7.6), 150 mM NaCl, 1% NP-40, 1% sodium deoxycholate, 0.1% SDS) supplemented with PMSF, Protease and Phosphatase inhibitor cocktail (Thermo Scientific), according to the vendor's indications. Extracts were kept on ice, and the samples were flowed through a needle to increase protein yield. Protein extracts were centrifuged at 14,000×g for 15 min to separate the proteins (supernatant) from the cellular debris (pellet). The concentration of protein in the extracts was measured with a Bradford protein assay. 30 μg of total protein extracts were loaded onto a 10% Mini-PROTEAN® TGX™ Precast Gel (BioRad, Hercules, Calif., USA). For particle characterization, 30 μg of Jukat cell extract, 1 million LLV and NPS were loaded onto the gel (FIG. 29C) (LLV were coated using 150 μg of cell membrane proteins). For phosphorylated VE-cadherin, VE-cadherin, 1.5×10⁶ HUVECs were plated onto fibronectin-coated 10-mm cell culture dishes, with or without medium containing TNFα (25 ng/mL). Then, 90×10⁷ NPS, J774-LLV and Jurkat-LLV, 1.5×10⁶ Jurkat cells were added to the media for 15 min (FIG. 32A). The proteins from the gels were blotted on a PVDF membrane using a BioRad Trans-Blot® Turbo™ Transfer Starter System according to the vendor's instructions. After 2 hrs blocking solution (Tris-Buffered Saline 0.1% Tween 20, 5% Blotting Grade Blocker Non Fat Dry Milk; BioRad) the membranes were hybridized with primary antibody [Anti VE-cadherin phospho ab22775 from Abcam; Anti-VE-cadherin ab33168 from Abcam; and Anti-GAPDH sc-137179 from Santa Cruz Biotechnology, Dallas, Tex., USA)]. Antibodies were used according to vendor's indications. For detection we used the Pierce ECL Western Blotting Substrate (Pierce, Waltham, Mass., USA) and the BioRadChemiDoc™ MP System (BioRad).

Animal Care.

Animal studies were conducted in accordance with institutional guidelines of the Animal Welfare Act and the Guide for the Care and Use of Laboratory Animals following approved protocols. Female 8-9 week old BALB/c mice were purchased from Charles River Laboratories (Boston, Mass., USA) and maintained using previously established protocols. Mouse breast cancer tumors were established using a single injection of 2×10⁵ 4 T1-luc2-tdTomato Bioware® Ultra Red from PerkinElmer (Waltham, Mass., USA) into the mammary fat pad. At pre-determined times, animals' images were acquired using an IVIS 200 imaging system (Perkin Elmer). Tumors were determined as established upon reaching a size of 0.8 cm³.

Intravital Microscopy Imaging.

Animals were anesthetized using isoflurane. After removing hair, the tumor mass was exposed under the microscope. 40 μL of FITC 70 kDa dextran solution was injected endovenously to maximize the definition and resolution of the vascular bed. In 3 animals per experimental group, 1 billion NPS or LLV were injected. Dextran and particles were systemically administered through the retro-orbital venous plexus. To analyze tumoritropic accumulation and binding stability, the animals were imaged after 1 hr and monitored for 2 hrs after particle injection. To evaluate the dextran extravasation time course, 3 mice per point were used and 5 different fields were analyzed per mouse. Images were filmed and collected for 45 min after particle injection. Dextran diffusion images were taken from the last frames of the IVM movies. Fluorescence intensity was quantified using ND2 software from Nikon.

Statistical Analysis.

Statistical analyses were calculated using Prism GraphPad v. 6.0. All studies were the result of a minimum of three biological replicates unless stated. Statistics for the immunofluorescence intensity of VE-cadherin expression was analyzed using a one-way ANOVA with a Tukey post-test comparing means. Statistics for dextran extravasation was analyzed using a two-way ANOVA with a Bonferroni post-test.

Results

Surface Characterization of Leukolike Vectors.

LLV were assembled using 1 μm discoidal NPS as previously reported. Briefly, LLV were fabricated using cellular membranes purified from human T-cells (Jurkat) or murine macrophages (J774) to minimize reactivity and closely mimic the biological vasculature activity that will be tested in vitro (i.e., human) and in vivo (i.e., murine), respectively. The membrane coating on the NPS surface was stabilized using electrostatic interactions between the negatively charged cellular membrane and the positively charged NPS, previously modified with (3-Aminopropyl) triethoxysilane (APTES). Scanning electron microscope micrographs revealed uniform membrane coating on the LLV surface with minimal exposure of the underlying nanopores. Zeta potential analysis demonstrated a positive charge after functionalization with APTES, while coating the NPS core with cellular membrane proteins resulted in a negative surface charge for both LLV formulations. This result was in accordance with the negative surface charge of native leukocytes.

Particle Characterization.

Next, fluorescent microscopy revealed the homogenous distribution of lymphocyte function-associated antigen 1 (LFA-1) and macrophage-1 antigen (Mac-1) adhesive proteins on the particle surface for both Jurkat LLV and J774 LLV. Their presence was further validated through western blot analysis and flow cytometry. These proteins have previously been shown to be fundamental in the activation of ICAM-1 expression on endothelial cells. To assess their role in the adhesion of LLV towards an inflamed endothelium, human umbilical vein endothelial cells (HUVEC) were treated with anti-LFA-1 LLV and anti-Mac-1 LLV under physiological flow conditions and compared to LLV. Our data revealed that compared to LLV, both anti-LFA-1 and anti-Mac-1 LLV resulted in decreased adhesion to the endothelial cells, confirming that both of these proteins participate in the interaction with inflamed vasculature). Furthermore, it was observed that the blocking of LFA-1 alone resulted in a significant inhibition of particle accumulation relative to Mac-1-blocked LLV. A similar phenomenon was observed in vivo using intravital microscopy by administering LLV, anti-LFA-1 LLV, and anti-Mac-1 LLV to BALB/c 4T1 breast cancer tumor-bearing mice. Blocking LFA-1 and Mac-1 both demonstrated a decrease in particle accumulation at tumor vasculature, with LFA-1 representing a significant decrease compared to LLV.

In addition, flow cytometry revealed post-translational modifications of adhesive proteins were maintained on the LLV surface as demonstrated by wheat germ agglutinin staining. The addition of the coating was also found to display minimal changes in particle size as demonstrated by dynamic light scattering and SEM images revealed a lack of particle aggregation following coating. This data provides a general physical, chemical, and biological characterization of the system, exhibiting the successful transfer of biological material onto synthetic particles and indicating the presence of the machinery necessary to adhere and activate the ICAM-1 pathway in inflamed endothelium.

ICAM-1 Pathway Activation.

Previously, we demonstrated that LLV is capable of inducing ICAM-1 clustering. Herein, we focused our attention to assess if this phenomenon was effectively followed by the activation of ICAM-1 pathway and determine its implication in terms of vascular permeability. All experiments were performed under flow on an inflamed endothelial monolayer developed using HUVEC activated with tumor necrosis factor alpha (TNF-α) treatment for 24 h. This model has been extensively used to investigate particle adhesion in flow dynamics. In these experimental conditions, endothelial cells overexpress ICAM-1, as shown in. Following a 10 min perfusion of particles at a rate of 0.1 dyn/cm², LLV preferentially accumulated at the cell-cell border, while NPS distributed more homogeneously on the surface of the cells. This finding suggested that the LLV preferentially adhered at cell edges and revealed that 23% more LLV localized at the cell borders when compared to NPS. Additionally, literature and the inventors' previous work demonstrated that the border of inflamed endothelial cells is predominantly enriched with ICAM-1 to engage surface interactions with circulating leukocytes.

In nature, the activation of the ICAM-1 pathway by leukocytes induces an increase in the intracellular concentration of Ca²⁺. To measure the changes in Ca²⁺ production following treatment with LLV, a combination of fluorometric analysis and live microscopy was used on a HUVEC monolayer. Increases in the cytoplasmic levels of Ca²⁺ were observed as quickly as 15 sec following interaction of LLV with inflamed endothelium. This finding corroborated results obtained previously in literature describing leukocyte extravasation.

Adhesion Proprieties and Effect on Calcium Signaling in Inflamed Endothelium.

Furthermore, ICAM-1 pathway activation involves the phosphorylation of protein kinase C alpha (PKCα) that, in turn, phosphorylates VE-cadherin, leading to its membrane displacement and the partial disruption of the endothelial intercellular junction. VE-cadherin is responsible for maintaining the endothelial monolayer's continuity and barrier function. Western blot analysis was used to assess the phosphorylation levels of VE-cadherin and PKCα on TNFα-activated HUVEC following treatment with LLV or NPS, while an untreated control and leukocytes (i.e., Jurkat T-cells) were used as negative and positive control, respectively. The analysis revealed that VE-cadherin phosphorylated protein (VE-cadherin-P) was 2.5-fold higher in LLV-treated HUVEC than in untreated cells, while the level of VE-cadherin-P slightly increased in NPS-treated cells, maintaining basal levels of phosphorylation similar to the controls. On the other hand, VE-cadherin-P expression was 1.5-fold higher in leukocyte-treated HUVEC than in controls, indicating that LLV retained the critical biological determinants necessary to induce VE-cadherin phosphorylation, while no significant changes occurred in total VE-cadherin protein expression after treatment. Similarly compared to an untreated control, endothelial cells treated with LLV and leukocytes increased their basal expression of PKCα phosphorylated protein (PKCα-P). The phosphorylation of these two important mediators represents a critical step in the functional down-regulation of VE-cadherin as it determines its cytoplasmic displacement from the edge of endothelial cells.

ICAM1 Pathway Activation.

VE-cadherin displacement from the membrane has previously been reported as an effect produced by leukocytes on endothelial cells after activation of the ICAM-1 pathway. This phenomenon was evaluated through fluorescence microscopy following particle flow, using similar experimental settings as described above. Under conditions that mimic capillary flow in vitro, inflamed HUVEC monolayers were exposed for 30 min. to leukocytes, NPS, or LLV. VE-cadherin expression along the cell perimeter was then analyzed by immunofluorescence. In comparison to untreated and NPS-treated cells, VE-cadherin expression decreased significantly (p<0.0001) in the group treated with LLV and leukocytes (p<0.0001).

Representative immunofluorescence images acquired following treatment. These results can also be observed in a tridimensional fluorescent analysis on the acquired images and by plotting the fluorescence intensity profile of the cell perimeter in polar coordinates. Conversely, VE-cadherin was only slightly decreased in non-inflamed endothelium after exposure to LLV. Collectively, these data confirm the specificity of the disclosed biomimetic delivery platform towards inflamed endothelia, and highlight its ability to actively trigger the ICAM-1 pathway. In particular, the proteolipid coating applied on the surface of the particles was effective in favoring VE-Cadherin phosphorylation and displacement, inhibiting the intercellular connections between the cells composing the monolayer.

LLV Targeting and Bioactivity In Vivo.

The advantages of LLV for targeting tumor-associated vasculature and in increasing its permeability were examined in an orthotopic 4T1 breast cancer tumor model. Ten days after tumor establishment, mice were treated with either NPS or LLV, followed by a single injection of a 70-kDa fluorescein isothiocyanate-dextran tracer (3% wt./vol.) to define tumor vasculature. The membrane coating applied on the LLV increased the targeting potential when compared with NPS, concurring with previously published results obtained in a melanoma model. In an attempt to shed light on the spatiotemporal mechanics of LLV interaction with tumor endothelium, a time-dependent evaluation of particle binding to the endothelium was performed at 1- and 2-hrs post-particle injection. Specifically, random sections of the tumor vasculature were assessed for the ability of particles to: 1) establish new binding events (in red), 2) firmly adhere to the tumor vasculature (in yellow), and 3) detach from the vessel wall (in white). The LLV and NPS showed similar properties in interacting with the tumor-associated vasculature, likely a result of the particle shape strategically designed to favor margination in the tumor capillaries. More so, the application of the leukocyte coating onto the NPS resulted in a 2.16-fold reduction in LLV detachment). These data suggested that the adhesion proteins on the LLV surface played an active role in the adhesion to the vessel wall and that key leukocyte proteins remain functional even after contact with the biological surface.

Intravital Microscopy Analysis of LLV Tumor Endothelium Targeting and Binding Stability.

To further investigate the ability of LLV to firmly adhere to an inflamed tumor vessel wall in vivo, a novel analytical tool was developed by merging consecutive frames obtained by intravital microscopy (IVM) movies into one image. Thus, the time course experiment was resolved into a series of single images in which the fluorescence of the LLV and NPS indicated the particle positions in the different frames. When these positions were projected onto a Cartesian coordinate system as a function of time, firmly bound LLV appeared as a straight horizontal or vertical line according to their respective X and Y coordinates, while NPS appeared as slanted lines, indicating reduced adhesion. Furthermore, slope calculations demonstrated the average velocity for traveling NPS remained at 9.8 μm/sec while LLV remained at 0 μm/sec, suggesting stable adhesion.

To gain further insights into the permeability of tumor vasculature following exposure with LLV, we measured the time-dependent extravasation of the intravenously administered fluorescent tracer, 70-kDa dextran. Using IVM, movie frames of the sub-endothelial tumor space were collected 1, 5, 30 and 45 min. after dextran administration. IVM images showed a linear increase in dextran extravasation in mice treated with LLV and NPS. However, at 45 min after treatment, dextran extravasation was more than 35% higher in LLV-treated mice compared to NPS-treated. This phenomenon was further confirmed by developing an intensity map of representative sections of the sub-endothelial space where the color code indicated a prominent extravasation of the fluorescent dye after 45 min. To analyze the penetration potential of dextran into the subendothelial space, a subsection beginning at the vessel and covering the subendothelial space was analyzed. This confirmed that LLV modulated the endothelial barrier, allowing the dextran to penetrate deeper into the subendothelial space and serves as a representation of how therapeutics (i.e., particles >70 kDa) can penetrate into the subendothelial space following LLV adhesion. In addition, the preferential accumulation of LLV at the tumor vasculature can further benefit from the working mechanism of NPS and deliver larger therapeutic agents through the degradation of the silicon core. Together, this data demonstrates that the leukocyte membrane coating enhances diffusion through the tumor vasculature in vivo by engaging specific surface interactions with the endothelial cells.

Summary

The last decade has seen the emergence of biomimetic strategies as promising alternatives to drug delivery platforms based on synthetic materials and the exploitation of the EPR effect. LLV have been fabricated based on the fusion of synthetic, modifiable NPS and purified leukocyte cell membrane. This coating has previously been demonstrated as retaining the properties portrayed by NPS, as demonstrated by the loading and release of model payloads (e.g., doxorubicin and albumin). In this example, it was shown that the coating did not interfere with the margination properties of NPS but rather enhanced the particle interaction with tumor blood vessels, providing a synergistic effect that results in superior targeting and firm adhesion. Additionally, the inventors have demonstrated that the coating could molecularly interact with the surface of the cell. Specifically, purified leukocyte plasma membranes grafted on the NPS surface efficiently activate the endothelial receptor ICAM-1 pathway, resulting in increased vascular permeability through the phosphorylation of VE-cadherin. Furthermore, in vivo studies demonstrated that this approach enhanced the targeting properties, promoted firm adhesion to the tumor vasculature, and increased tumor perfusion. This work provides further confirmation for the implementation of synthetic materials with biological components in overcoming the current limitation in nanocarrier fabrication. Moreover, it improves upon the existing treatment modalities for diseases characterized by leukocyte infiltration. From this work, it has been shown that the cell membrane isolated and applied onto NPS at least partially preserves its biological activity. These results demonstrate that the biomolecular properties remain functional, thereby highlighting an alternative approach to current nanocarrier design.

### REFERENCES

The following references, to the extent that they provide exemplary procedural or other details supplementary to those set forth herein, are specifically incorporated herein by reference:
ALLEN, T, “Liposomal drug formulations,” Drugs, 56:747-756 (1998).
ALVAREZ-LORENZO, C and CONCHEIRO, A, “Bioinspired drug delivery systems,” Curr. Opin. Biotechnol., 24:1167-1173 (2013).
ANSELMO, A C et al., “Platelet-like nanoparticles: mimicking shape, flexibility, and surface biology of platelets to target vascular injuries,” ACS Nano 8:11243-11253 (2014).
ARORA, H C et al., “Nanocarriers enhance doxorubicin uptake in drug-resistant ovarian cancer cells,” Cancer Res., 72:769-778 (2012).
ARROYO, A G et al., “Induction of tyrosine phosphorylation during ICAM-3 and LFA-1-mediated intercellular adhesion, and its regulation by the CD45 tyrosine phosphatase,” J. Cell Biol., 126:1277-1286 (1994).
AZZOPARDI, E A et al., “The enhanced permeability retention effect: a new paradigm for drug targeting in infection,” J. Antimicrob. Chemother., 68:257-274 (2013).
BARENHOLZ, Y, “Doxil®—The first FDA-approved nano-drug: Lessons learned,” J. Controlled Rel., 160:117-134 (2012).
BELLETTI, D et al., “Functionalization of liposomes: microscopical methods for preformulative screening,” J. Liposome Res., 1-7 (2014).
BENMERAH, A et al., “Nuclear functions for plasma membrane-associated proteins?” Traffic, 4:503-511 (2003).
BERNSDORFF, C et al., “Interaction of the anticancer agent Taxol™ (paclitaxel) with phospholipid bilayers,” J. Biomed. Mat. Res., 46:141-149 (1999).
BLANCO, E et al., “Principles of nanoparticle design for overcoming biological barriers to drug delivery,” Nature Biotechnol., 33:941-951 (2015).
BRETSCHER, M S, “Asymmetrical lipid bilayer structure for biological membranes,” Nature, 236:11-12 (1972).
BUDAI, L et al., “Liposomes for topical use: a physico-chemical comparison of vesicles prepared from egg or soy lecithin,” Scientia Pharmaceutica, 81:1151 (2013).
CHEN, X et al., “Inflamed leukocyte-mimetic nanoparticles for molecular imaging of inflammation,” Biomaterials, 32:7651-7661 (2011).
CHENG, Z et al., “Multifunctional nanoparticles: cost versus benefit of adding targeting and imaging capabilities,” Science, 338:903-910 (2012).
CHOW, TS, “Nanoscale surface roughness and particle adhesion on structured substrates,” Nanotechnology, 18:115713 (2007).
CLARKE, D T W and MCMILLAN, N A J, “Gene delivery: Cell-specific therapy on target,” Nat. Nano, 9:568-569 (2014).
COPP, J A et al., “Clearance of pathological antibodies using biomimetic nanoparticles,” Proc. Natl. Acad. Sci. USA, 111:13481-13486 (2014).
CORBO, C, et al., “Proteomic profiling of a biomimetic drug delivery platform,” Curr. Drug Targets, 16(13):1540-1547 (2015).
COSCO, D et al., “Gemcitabine and tamoxifen-loaded liposomes as multidrug carriers for the treatment of breast cancer diseases,” Int. J. Pharmaceut., 422:229-237 (2012).
COUSSENS, LM and WERB, Z, “Inflammation and cancer,” Nature, 420:860-867 (2002).
DARSZON, A et al., “Reassembly of protein-lipid complexes into large bilayer vesicles: perspectives for membrane reconstitution,” Proc. Natl. Acad. Sci. USA, 77:239-243 (1980).
DAVIS, M E, “Nanoparticle therapeutics: an emerging treatment modality for cancer,” Nature Rev. Drug Discov., 7:771-782 (2008).
DE VISSER, K E et al., “Paradoxical roles of the immune system during cancer development,” Nat. Rev. Cancer, 6:24-37 (2006).
DEMETZOS, C, “Differential scanning calorimetry (DSC): a tool to study the thermal behavior of lipid bilayers and liposomal stability,” J. Liposome Res., 18:159-173 (2008).
DOSHI, N et al., “Platelet mimetic particles for targeting thrombi in flowing blood,” Adv. Mater., 24:3864-3869 (2012).
DURR, E et al., “Direct proteomic mapping of the lung microvascular endothelial cell surface in vivo and in cell culture,” Nature Biotechnol., 22:985-992 (2004).
FERRARI, M, “Cancer nanotechnology: opportunities and challenges,” Nature Rev. Cancer, 5:161-171 (2005).
FRANCHIMONT, D et al., “Glucocorticoids and inflammation revisited: the state of the art,” Neuroimmunomodulation, 10:247-260 (2002).
GELAIN, F et al., “Transplantation of nanostructured composite scaffolds results in the regeneration of chronically injured spinal cords,” ACS Nano, 5:227-236 (2010).
GEROMANOS, S J et al., “The detection, correlation, and comparison of peptide precursor and product ions from data independent LC-MS with data dependant LC-MS/MS,” Proteomics, 9:1683-1695 (2009).
GROSS, S et al., “Bioluminescence imaging of myeloperoxidase activity in vivo,” Nat. Med., 15:455-461 (2009).
GUTIÉRREZ MILLÁN, C et al., “Cell-based drug-delivery platforms,” Ther. Del., 3:25-41 (2012).
HAMMER, D A et al., “Leuko-polymersomes,” Faraday Discuss., 139:129-141 (2008).
HU, C-M J et al., ‘Marker-of-self’ functionalization of nanoscale particles through a top-down cellular membrane coating approach,” Nanoscale, 5:2664-2668 (2013).
HU, C-M J et al., “Erythrocyte membrane-camouflaged polymeric nanoparticles as a biomimetic delivery platform,” Proc. Natl. Acad. Sci. USA,
108:10980-10985 (2011).
HU, C-M J et al., “Nanoparticle biointerfacing by platelet membrane cloaking,” Nature, 526:118-121 (October 2015).
ISHIBASHI, M et al., “Integrin LFA-1 regulates cell adhesion via transient clutch formation,” Biochem. Biophys. Res. Comm., 464:459-466 (2015).
JAIN, MK and ZAKIM, D, “The spontaneous incorporation of proteins into preformed bilayers,” Biochim. Biophys. Acta (BBA)-Rev. Biomembr., 906:33-68 (1987).
KANGO, S et al., “Surface modification of inorganic nanoparticles for development of organic-inorganic nanocomposites-A review,” Progr. Polymer Sci., 38:1232-1261 (2013).
KARMALI, PP and SIMBERG, D, “Interactions of nanoparticles with plasma proteins: implication on clearance and toxicity of drug delivery systems,” Exp. Opin. Drug Deliv., 8:343-357 (2011).
KEEFE, A D et al., “Aptamers as therapeutics,” Nat. Rev. Drug Discov., 9:537-550 (2010).
KO, Y T et al., “Gene delivery into ischemic myocardium by double-targeted lipoplexes with anti-myosin antibody and TAT peptide,” Gene Ther., 16:52-59 (2008).
KUDGUS, R A et al., “Tuning pharmacokinetics and biodistribution of a targeted drug delivery system through incorporation of a passive targeting component,” Sci. Rep., 4:5669 (2014).
LAMMERS, T et al., “Tumour-targeted nanomedicines: principles and practice,” Brit. J. Cancer, 99:392-397 (2008).
LAOUINI, A et al., “Preparation, characterization and applications of liposomes: state of the art,” J. Coll. Sci. Biotechnol., 1:147-168 (2012).
LIANG, X et al., “Quantification of membrane and membrane-bound proteins in normal and malignant breast cancer cells isolated from the same patient with primary breast carcinoma,” J. Proteome Res., 5:2632-2641 (2006).
LIU, X et al., “Membrane proteomic analysis of pancreatic cancer cells,” J. Biomed. Sci., 17:74 (2010).
LODISH, H et al., “Molecular Cell Biology,” 4^{th} Ed. New York: W.H. Freeman (2000).
LORENZ, H M et al., “CD45 mAb induces cell adhesion in peripheral blood mononuclear cells via lymphocyte function-associated antigen-1 (LFA-1) and intercellular cell adhesion molecule 1 (ICAM-1),” Cell. Immunol., 147:110-128 (1993).
LUK, BT and ZHANG, L, “Cell membrane-camouflaged nanoparticles for drug delivery,” J. Contr. Rel., (2015).
LUND, R et al., “Efficient isolation and quantitative proteomic analysis of cancer cell plasma membrane proteins for identification of metastasis-associated cell surface markers,” J. Proteome Res., 8:3078-3090 (2009).
MANCONI, M et al., “Ex vivo skin delivery of diclofenac by transcutol containing liposomes and suggested mechanism of vesicle-skin interaction,” Eur. J. Pharmaceut. Biopharmaceut., 78:27-35 (2011).
MATHAES, R et al., “Influence of particle geometry and PEGylation on phagocytosis of particulate carriers,” Int. J. Pharmaceut., 465:159-164 (2014).
MEREGHETTI, P et al., “A Fourier transform infrared spectroscopy study of cell membrane domain modifications induced by docosahexaenoic acid,” Biochim. Biophys. Acta (BBA) 1840:3115-3122 (2014).
MILLAN, C G et al., “Drug, enzyme and peptide delivery using erythrocytes as carriers,” J. Contr. Rel., 95:27-49 (2004).
MINARDI, S. et al., “Evaluation of the osteoinductive potential of a bio-inspired scaffold mimicking the osteogenic niche, for bone augmentation,:” Biomaterials, (2015).
MITRAGOTRI, S et al., “Overcoming the challenges in administering biopharmaceuticals: formulation and delivery strategies,” Nature Rev. Drug Discov., 13:655-672 (2014).
MOURTAS, S et al., “Liposomal drugs dispersed in hydrogels: effect of liposome, drug and gel properties on drug release kinetics,” Colloids Surfaces B: Biointerfaces, 55:212-221 (2007).
MULLER, W A, “Mechanisms of leukocyte transendothelial migration,” Annu. Rev. Pathol., 6:323 (2011).
MURA, S et al., “Penetration enhancer-containing vesicles (PEVs) as carriers for cutaneous delivery of minoxidil,” Int. J. Pharmaceut., 380:72-79 (2009).
MURA, S et al., “Stimuli-responsive nanocarriers for drug delivery,” Nat. Mater., 12:991-1003 (2013).
NOGUEIRA, E et al., “Liposome and protein based stealth nanoparticles,” Faraday Disc., 166:417-429 (2013).
PARODI, A et al., “Bromelain surface modification increases the diffusion of silica nanoparticles in the tumor extracellular matrix,” ACS Nano, 8(10):9874-9883 (2014).
PARODI, A et al., “Synthetic nanoparticles functionalized with biomimetic leukocyte membranes possess cell-like functions,” Nature Nanotechnol., 8:61-68 (2013).
RABANEL, J-M et al., “Assessment of PEG on polymeric particles surface, a key step in drug carrier translation,” J. Controlled Rel., 185:71-87 (2014).
RAMACHANDRAN, S et al., “Cisplatin nanoliposomes for cancer therapy: AFM and fluorescence imaging of cisplatin encapsulation, stability, cellular uptake, and toxicity,” Langmuir, 22:8156-8162 (2006).
RIGAUD, J-L et al., “Reconstitution of membrane proteins into liposomes: application to energy-transducing membrane proteins,” Biochim. Biophys. Acta (BBA) Bioenergetics, 1231:223-246 (1995).
ROBBINS, G P et al., “Tunable leuko-polymersomes that adhere specifically to inflammatory markers,” Langmuir, 26:14089-14096 (2010).
SCHAAP, I A et al., “Effect of envelope proteins on the mechanical properties of influenza virus,” J. Biol. Chem., 287:41078-41088 (2012).
SEDDON, A M et al., “Membrane proteins, lipids and detergents: not just a soap opera,” Biochim. Biophys. Acta (BBA) Biomembranes, 1666:105-117 (2004).
SHERMAN, M B et al., “Removal of divalent cations induces structural transitions in red clover necrotic mosaic virus, revealing a potential mechanism for RNA release,” J. Virol., 80:10395-10406 (2006).
SIGAL, A et al., “The LFA-1 integrin supports rolling adhesions on ICAM-1 under physiological shear flow in a permissive cellular environment,” J. Immunol.,
165:442-452 (2000).
SILVA, J C et al., “Quantitative proteomic analysis by accurate mass retention time pairs,” Anal. Chem., 77:2187-2200 (2005).
SILVA, J C et al., “Absolute quantification of proteins by LCMSE a virtue of parallel MS acquisition,” Molec. Cell. Proteomics, 5:144-156 (2006).
SOTO-PANTOJA, D R et al., “Leukocyte surface antigen CD47,” UCSD Molecule Pages, 2(1):19-36 (2013).
SRIRAMAN, SK and TORCHILIN, VP, “Recent advances with liposomes as drug carriers,” in Advanced Biomaterials and Biodevices (eds A. Tiwari and A. N. Nordin), John Wiley & Sons, Inc., Hoboken, N.J., USA. doi: 10.1002/9781118774052.ch3 79-119 (2014).
SVENSON, S, “Clinical translation of nanomedicines,” Curr. Opin. Solid State Mat. Sci., 16:287-294 (2012).
SYKES, E A et al., “Investigating the impact of nanoparticle size on active and passive tumor targeting efficiency,” ACS Nano, 8:5696-5706 (2014).
SZOKA, F and PAPAHADJOPOULOS, D, “Procedure for preparation of liposomes with large internal aqueous space and high capture by reverse-phase evaporation,” Proc. Natl. Acad. Sci. USA, 75:4194-4198 (1978).
TASCIOTTI, E et al., “Mesoporous silicon particles as a multistage delivery system for imaging and therapeutic applications,” Nature Nanotechnol., 3:151-157 (2008).
TERMSARASAB, U et al., “Polyethylene glycol-modified arachidyl chitosan-based nanoparticles for prolonged blood circulation of doxorubicin,” Int. J. Pharmaceut., 464:127-134 (2014).
TOLEDANO-FURMAN, N E et al., “Reconstructed stem cell nanoghosts: a natural tumor targeting platform,” Nano Lett., 13:3248-3255 (2013).
TOMITA, T et al., “Influence of membrane fluidity on the assembly of Staphylococcus aureus alpha-toxin, a channel-forming protein, in liposome membrane,” J. Biol. Chem., 267:13391-13397 (1992).
TORCHILIN, VP, “Multifunctional, stimuli-sensitive nanoparticulate systems for drug delivery,” Nat. Rev. Drug Discov., 13:813-827 (2014).
VARKOUHI, A K et al., “Endosomal escape pathways for delivery of biologicals,” J. Controlled Rel., 151:220-228 (2011).
WORTHYLAKE, RA and BURRIDGE, K, “Leukocyte transendothelial migration: orchestrating the underlying molecular machinery,” Curr. Opin. Cell Biol., 13:569-577 (2001).
YOO, J-W et al., “Bio-inspired, bioengineered and biomimetic drug delivery carriers,” Nature Rev. Drug Discov., 10:521-535 (2011).
ZARBOCK, A et al., “Leukocyte ligands for endothelial selectins: specialized glycoconjugates that mediate rolling and signaling under flow,” Blood, 118:6743-6751 (2011).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are specifically incorporated herein by reference to the same extent as if each reference was individually and specifically indicated to be incorporated by reference, and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the concept, spirit and scope of the invention. More specifically, it will be apparent that certain agents that are chemically- or physiologically-related may be substituted for the agents described herein while the same or similar results would be achieved. All such similar substitutes and modifications apparent to those ordinarily skilled in the art are deemed to be within the spirit, scope, and concept of the invention as defined by the appended claims.

What is claimed is:

1. A drug delivery composition comprising a population of biomimetic proteolipid nanovesicles composed of synthetic phospholipids and cholesterol, enriched of leukocyte membrane fragments, and surrounding an aqueous core.

2. The drug delivery composition of claim 1, wherein the proteolipid nanovesicles comprise at least one self-tolerance protein or active fragment thereof on their surface, such as CD-45, CD-47, or MHC-1.

3. The drug delivery composition of claim 1, further comprising at least one therapeutic agent.

4. The drug delivery composition of claim 1, wherein the leukocyte membrane fragments are derived from human leukocyte plasma membranes.

5. The drug delivery composition of claim 3, wherein the at least one therapeutic agent is selected from the group consisting of an immune-stimulating agent, a tumor growth inhibitor, a protein, a peptide, an RNA molecule, a DNA molecule, an siRNA molecule, a RNAi molecule, an ssRNA molecule, a growth factor, an enzyme inhibitor, a binding protein, a blocking peptide, and any combination thereof.

6. The drug delivery composition of claim 1, wherein the proteolipid nanovesicles are adapted configured to release the at least one therapeutic agent in response to an external stimulus, in response to a change in the environment of the population of biomimetic proteolipid nanovesicles, or as a result of degradation of the proteolipid nanovesicles.

7. The drug delivery composition of claim 1, wherein degradation of the population of biomimetic proteolipid nanovesicles occurs via enzyme-facilitated biodegradation of one or more of the phospholipids or the cholesterol comprising them.

8. The drug delivery composition of claim 1, wherein the leukocyte membrane fragments comprise at least one cellular-targeting moiety.

9. The drug delivery composition of claim 8, wherein the at least one cellular-targeting moiety is selected from the group consisting of a chemically-targeting moiety, a physically-targeting moiety, a geometrically-targeting moiety, a ligand, a ligand-binding moiety, a receptor, a receptor-binding moiety, an antibody or an antigen-binding fragment thereof, and any combination thereof.

10. The drug delivery composition of claim 8, wherein the at least a first cellular-targeting moiety comprises a plurality of distinct antigenic ligands that elicit one or more target-specific immune responses in a mammalian host cell that is contacted with the population of nanovesicles.

11. The drug delivery composition of claim 1, further comprising a diagnostic agent.

12. The drug delivery composition of claim 11, wherein the a diagnostic reagent is selected from the group consisting of an imaging agent, a contrast agent, a fluorescent label, a radiolabel, a magnetic resonance imaging label, a spin label, and any combination thereof.

13. The drug delivery composition of claim 1, comprising a chemically-targeting moiety that is disposed on the surface of the proteolipid nanovesicles, and that comprises a ligand, a dendrimer, an oligomer, an aptamer, a binding protein, an antibody, an antigen-binding fragment thereof, a biomolecule, or any combination thereof.

14. The drug delivery composition of of claim 1, wherein the biomimetic proteolipid nanovesicles are about 100 to about 1000 nm in average diameter.

15. The drug delivery composition of claim 1, wherein the synthetic phospholipids are selected from the group consisting of phosphatidylcholine, egg phosphatidic acid, 1,2-dioleoyl-sn-glycerophosphocholine (DOPC), 1,2-dioleoyl-sn-glycerophosphoethanolamine (DOPE), 1,2-dipalmitoyl-sn-glycerophosphocholine (DPPC), 1,2-distearoyl-sn-glycerophosphocholine (DSPC), and any combination thereof.

16. The drug delivery composition of claim 5, wherein the siRNA molecule is specific for a mammalian gene selected from the group consisting of BRAF, MEK, ERK1, and ERK2.

17. The drug delivery composition of claim 1, wherein the lipid-to-protein ratio is from about 160-to-5 (wt./wt.) to about 300-to-1 (wt./wt.).

18. A population of isolated mammalian host cells comprising the drug delivery composition of claim 1.

19. A pharmaceutical formulation comprising the drug delivery composition of claim 1, and a pharmaceutically-acceptable buffer, diluent, excipient, or vehicle.

20. A kit comprising the drug delivery composition of claim 1, and instructions for administering the composition to a mammal in need thereof, as part of a regimen for the prevention, diagnosis, treatment, or amelioration of one or more symptoms of a disease, a dysfunction, an abnormal condition, or a trauma in the mammal.

21. A method for providing one or more active agents to a population of cells within the body of an animal, comprising administering to the animal an amount of the drug delivery composition of claim 1, for a time effective to provide the one or more active agents to the population of cells within the body of the animal.

22. The method of claim 21, wherein the animal is at risk for developing, is suspected of having, or is diagnosed with a tumor or a cancer.

23. A method of administering a diagnostic, therapeutic, or prophylactic agent to one or more cells, tissues, organs, or systems of a mammalian subject in need thereof, comprising administering to the subject an effective amount of the drug delivery composition of claim 1.

24. The method of claim 23, wherein the drug delivery composition comprises a therapeutic agent selected from the group consisting of a siRNA, an ssRNA, an RNAi, a DNA, an RNA, and any combination thereof.

25. The method of claim 23, wherein the drug delivery composition further comprises at least a first chemotherapeutic agent.

26. The method of claim 25, wherein the at least a first chemotherapeutic agent comprises paclitaxel or dexamethasone.
